(19) United States
(12) Patent Application Publication
(10) Pub. No.: US 2002/0090692 A1
(43) Pub. Date: Jul. 11, 2002

(54) Novel polynucleotides and polypeptides encoded thereby

(76) Inventors: Sudhirdas K. Prayaga, O'Fallon, MO (US); Corine Vernet, North Branford, CT (US); Richard A. Shimkets, West Haven, CT (US); Catherine E. Burgess, Wethersfield, CT (US); Kimberly A. Spytek, New Haven, CT (US); Velizar Tchernev, Branford, CT (US); Valerie Gerlach, Branford, CT (US); John R. MacDougall, Hamden, CT (US); Isabelle Millet, Milford, CT (US); David J. Stone, Guilford, CT (US); Karen Ellerman, Branford, CT (US)

Correspondence Address: Ivor R. Elrifi, MINTZ, LEVIN, COHN, FERRIS,, GLOVSKY AND POPEO, P.C., One Financial Center, Boston, MA, 02111, US

(21) Appl. No.: 09/974,294

(22) Filed: Oct. 10, 2001

Related U.S. Application Data
(63) Continuation-in-part of application No. 09/882,263, filed on Jun. 15, 2001.
Application No. 09/882,263 is a continuation-in-part of application No. 09/672,665, filed on Sep. 28, 2000.
(60) Provisional application No. 60/156,745, filed on Sep. 30, 1999.
(60) Provisional application No. 60/158,942, filed on Oct. 6, 1999.
(60) Provisional application No. 60/169,344, filed on Dec. 6, 1999.
(60) Provisional application No. 60/215,048, filed on Jun. 29, 2000.
(60) Provisional application No. 60/239,411, filed on Oct. 10, 2000.

(51) Int. Cl. C12N 9/00; C07H 21/04; C12P 21/02; C12N 5/06

(52) U.S. Cl. 435/183; 435/69.1; 435/325; 435/320.1; 536/23.2

(57) ABSTRACT

The present invention provides novel polypeptides, termed PTMAX polypeptides, as well as polynucleotides encoding PTMAX polypeptides and antibodies that immunospecifically bind to PTMAX or a derivative, variant, mutant, or fragment of the PTMAX polypeptide, polynucleotide or antibody. The invention additionally provides methods in which the PTMAX polypeptide, polynucleotide and antibody are used in detection and treatment of a broad range of pathological states, as well as to other uses.

### RELATED APPLICATIONS

This application claims priority to U.S. Ser. No. 09/672,665 filed Sep. 28, 2000; U.S. Ser. No. 09/882,263 filed Jun. 15, 2001 U.S. Ser. No. 60/156,745 filed Sep. 30, 1999; U.S. Ser. No. 60/158,942 filed Oct. 6, 1999; U.S. Ser. No. 60/159,248 filed Oct. 13, 1999; U.S. Ser. No. 60/169,344 filed Dec. 6, 1999; U.S. Ser. No. 60/215,048 filed Jun. 29, 2000; and U.S. Ser. No. 60/239,411 filed Oct. 10, 2000 which are incorporated herein by reference in their entirety.

### BACKGROUND OF THE INVENTION

The invention relates in general to nucleic acids and polypeptides; more particularly it relates to polynucleotides expressed in the thymus gland and other tissues, and polypeptides encoded by such polynucleotides, as well as vectors, host cells, antibodies and recombinant methods for producing the polypeptides and polynucleotides.

### SUMMARY OF THE INVENTION

The present invention is based in part on the discovery of novel polynucleotide sequences. These human nucleic acids and polypeptides encoded thereby are collectively referred to herein as “PTMAX”.

Accordingly, in one aspect, the invention provides an isolated nucleic acid molecule that encodes a novel polypeptide, or a fragment, homolog, analog or derivative thereof. The nucleic acid can include, e.g., a nucleic acid sequence encoding a polypeptide at least 85% identical to a polypeptide comprising the amino acid sequences of SEQ ID NO:2n, wherein n is an integer between 1-10, or a polypeptide that is a fragment, homolog, analog or derivative thereof. The nucleic acid can include, e.g., one or more fragments from genomic DNA, or a cDNA molecule, or an RNA molecule. In particular embodiments, the nucleic acid molecule may include the sequence of any of SEQ ID NO:2n-1, wherein n is an integer between 1-10. These polypeptides and nucleic acids are related to a prothymosin alpha, an oncostatin or a nerve growth factor sequence, as disclosed herein.

Also included in the invention is a vector containing one or more of the nucleic acids described herein, and a cell containing the vectors or nucleic acids described herein.

The invention is also directed to host cells transformed with a vector comprising any of the nucleic acid molecules described above.

In another aspect, the invention includes a pharmaceutical composition that includes a PTMAX nucleic acid and a pharmaceutically acceptable carrier or diluent.

In a further aspect, the invention includes a substantially purified PTMAX polypeptide, e.g., any of the PTMAX polypeptides encoded by a PTMAX nucleic acid, and fragments, homologs, analogs, and derivatives thereof. The invention also includes a pharmaceutical composition that includes a PTMAX polypeptide and a pharmaceutically acceptable carrier or diluent.

In a still further aspect, the invention provides an antibody that binds specifically to a PTMAX polypeptide. The antibody can be, e.g., a monoclonal or polyclonal antibody, and fragments, homologs, analogs, and derivatives thereof. The invention also includes a pharmaceutical composition including PTMAX antibody and a pharmaceutically acceptable carrier or diluent. The invention is also directed to isolated antibodies that bind to an epitope on a polypeptide encoded by any of the nucleic acid molecules described above.

The invention also includes kits comprising any of the pharmaceutical compositions described above.

The invention further provides a method for producing a PTMAX polypeptide by providing a cell containing a PTMAX nucleic acid, e.g., a vector that includes a PTMAX nucleic acid, and culturing the cell under conditions sufficient to express the PTMAX polypeptide encoded by the nucleic acid. The expressed PTMAX polypeptide is then recovered from the cell. Preferably, the cell produces little or no endogenous PTMAX polypeptide. The cell can be, e.g., a prokaryotic cell or eukaryotic cell.

The invention is also directed to methods of identifying a PTMAX polypeptide or nucleic acids in a sample by contacting the sample with a compound that specifically binds to the polypeptide or nucleic acid, and detecting complex formation, if present.

The invention further provides methods of identifying a compound that modulates the activity of a PTMAX polypeptide by contacting PTMAX polypeptide with a compound and determining whether the PTMAX polypeptide activity is modified.

The invention is also directed to compounds that modulate PTMAX polypeptide activity identified by contacting a PTMAX polypeptide with the compound and determining whether the compound modifies activity of the PTMAX polypeptide, binds to the PTMAX polypeptide, or binds to a nucleic acid molecule encoding a PTMAX polypeptide.

In another aspect, the invention provides a method of determining the presence of or predisposition of a PTMAX-associated disorder in a subject. The method includes providing a sample from the subject and measuring the amount of PTMAX polypeptide in the subject sample. The amount of PTMAX polypeptide in the subject sample is then compared to the amount of PTMAX polypeptide in a control sample. An alteration in the amount of PTMAX polypeptide in the subject protein sample relative to the amount of PTMAX polypeptide in the control protein sample indicates the subject has pathology related to a dysfunction in the immune system, a tissue proliferation-associated condition, or a neurological disorder. A control sample is preferably taken from a matched individual, i.e., an individual of similar age, sex, or other general condition but who is not suspected of having a dysfunction in the immune system, a tissue proliferation-associated condition, or a neurological disorder. Alternatively, the control sample may be taken from the subject at a time when the subject is not suspected of having a dysfunction in the immune system, a tissue proliferation-associated condition, or a neurological disorder. In some embodiments, the PTMAX polypeptide is detected using a PTMAX antibody.

In a further aspect, the invention provides a method of determining the presence of, or predisposition to a PTMAX-associated disorder in a subject. The method includes providing a nucleic acid sample, e.g., RNA or DNA, or both, from the subject and measuring the amount of the PTMAX nucleic acid in the subject nucleic acid sample. The amount of PTMAX nucleic acid sample in the subject nucleic acid is then compared to the amount of PTMAX nucleic acid in a control sample. An alteration in the amount of PTMAX nucleic acid in the sample relative to the amount of PTMAX in the control sample indicates the subject has a dysfunction in the immune system, a tissue proliferation-associated condition, or a neurological disorder.

In a still further aspect, the invention provides a method of treating or preventing or delaying a PTMAX-associated disorder. The method includes administering to a subject in which such treatment or prevention or delay is desired a PTMAX nucleic acid, a PTMAX polypeptide, or a PTMAX antibody in an amount sufficient to treat, prevent, or delay an immune disorder, a tissue proliferation-associated disorder, or a neurological disorder in the subject.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The invention provides novel polypeptides and nucleotides encoded thereby. Included in the invention are ten novel nucleic acid sequences and their encoded polypeptides. The sequences are collectively referred to as “PTMAX nucleic acids” or “PTMAX polynucleotides” and the corresponding encoded polypeptide is referred to as a “PTMAX polypeptide” or “PTMAX protein”. For example, a PTMAX nucleic acid according to the invention is a nucleic acid including a PTMAX nucleic acid, and a PTMAX polypeptide according to the invention is a polypeptide that includes the amino acid sequence of a PTMAX polypeptide. Unless indicated otherwise, “PTMAX” is meant to refer to any of the novel sequences disclosed herein.

Table 1 provides a summary of the PTMAX nucleic acids and their encoded polypeptides.

Column 1 of Table 1, entitled “PTMAX No.”, denotes a PTMAX number assigned to a nucleic acid according to the invention.

Column 2 of Table 1, entitled “Clone Identification Number” provides a second identification number for the indicated PTMAX.

Column 3 of Table 1, entitled “Tissue of Origin of the Clone”, indicates the tissue in which the indicated PTMAX nucleic acid is expressed.

Columns 4-9 of Table 1 describe structural information as indicated for the indicated PTMAX nucleic acids and polypeptides.

Column 10 of Table 1, entitled “Protein Similarity” lists previously described proteins that are related to polypeptides encoded by the indicated PTMAX. Genbank identifiers for the previously described proteins are provided. These can be retrieved from

http://www.ncbi.nlm.nih.gov/.

Column 11 of Table 1, entitled“Signal Peptide Cleavage Site” indicates the putative nucleotide position where the signal peptide is cleaved as determined by SignalP.

Column 12 of Table 1, entitled “Cellular Localization” indicates the putative cellular localization of the indicated PTMAX polypeptides.

TABLE 1

| | | | | Open | | | | Signal | |
|---|---|---|---|---|---|---|---|---|---|
| | Clone | Tissue of | | Reading | | Calculated | | Peptide | |
| TMAX | Identification | Origin of | Nucleotide | Frame | AA | Molecular | | Cleavage | Cellular |
| o. | Number | the Clone | Length | (nt) | Residues | Weight | Protein Similarity | Site (nt) | Localization |
| 1 | AC009485_A | Genomic | 327 | 1-327 | 109 | 11909.9 | Ptnr:REMTREMBL- | None | Cytoplasm |
| | | | | | | | ACC:G190372, human | | |
| | | | | | | | prothymosin alpha | | |
| | | | | | | | pseudogene;Pntr:SPTRE | | |
| | | | | | | | MBL-ACC:Q15249, | | |
| | | | | | | | human prothymosin alpha | | |
| 2 | AC010175_A.0.1 | Genomic, | 555 | 1-342 | 114 | 12389.2 | ACC:AAA36485, human | None | Cytoplasm |
| | | placenta, | | | | | prothymosin-alpha | | |
| | | spleen | | | | | pseudogene; | | |
| 3 | AC010175_A.9.5 | Genomic, | 675 | 55-397 | 114 | 12481.4 | REMTREMBL- | None | Nucleus |
| | | placenta, | | | | | ACC:AAA36485, human | | |
| | | spleen | | | | | prothymosin-alpha | | |
| | | | | | | | pseudogene | | |
| 4 | AC009533_A | Genomic | 345 | 1-342 | 114 | 12390.2 | Ptnr:REMTREMBL- | None | Cytoplasm |
| | | | | | | | ACC:G190372, human | | |
| | | | | | | | prothymosin alpha | | |
| | | | | | | | pseudogene;Ptnr:SPTRE | | |
| | | | | | | | MBL-ACC:Q 15249, | | |
| | | | | | | | human prothymosin alpha | | |
| 5 | AL121585_A | Genomic | 501 | 134- | 109 | 12005.8 | ACC:g625274, | None | Cytoplasm |
| | | | | 460 | | | prothymosin alpha - | | |
| | | | | | | | human; ACC:g135833, | | |
| | | | | | | | prothymosin alpha - | | |
| | | | | | | | bovine | | |
| 6 | AC010175 | Genomic | 342 | 1-342 | 114 | 12389.2 | Human prothymosin alpha | None | Cytoplasm |
| 7 | AC010784-1 | Genomic | 324 | 1-324 | 108 | 11680.7 | Oncostatin A (Platelet | Betw. | plasma |
| | | | | | | | Factor 4 precursor) | Residues | membrane |
| | | | | | | | | 40 and 41: | |
| | | | | | | | | AEA- | |
| | | | | | | | | EE | |
| 8 | AL049825 | Genomic | 738 | 13-735 | 241 | 26958.5 | Nerve Growth Factor | | Extra- |
| | | | | | | | | | cellular or |
| | | | | | | | | | lysosome |
| | | | | | | | | | (lumen) |
| 9 | AL121585_da1 | Genomic | 345 | 10-339 | 110 | 12071.8 | Prothymosin alpha | None | Cytoplasm |
| 10 | AL121585_da2 | Genomic | 350 | 10-348 | 113 | 12348.2 | Prothymosin alpha | None | Cytoplasm |
| 11 | AL121585_da3 | Genomic | 497 | 134- | 110 | 12071.8 | Prothymosin alpha | None | Cytoplasm |
| | | | | 463 | | | | | |
| 12 | AL121585 | Genomic | 497 | 134- | 110 | N/A | Prothymosin alpha | None | Cytoplasm |
| | | | | 460 | | | | | |
| 13 | CG54101-06 | Genomic | 493 | 134- | 116 | N/A | Prothymosin alpha | None | Cytoplasm |
| | | | | 481 | | | | | |

Table 2 provides a cross reference to the assigned PTMAX number, clone identification number and sequence identification numbers (SEQ ID NOs.).

TABLE 2

| | | SEQ ID | |
|---|---|---|---|
| | Clone | NO | |
| | Identification | Nucleic | SEQ ID NO |
| PTMAX No. | Number | Acid | Polypeptide |
| 1 | AC009485_A | 1 | 2 |
| 2 | AC010175_A.0.1 | 3 | 4 |
| 3 | AC010175_A.9.5 | 5 | 6 |
| 4 | AC009533_A | 7 | 8 |
| 5 | AL121585_A | 9 | 10 |
| 6 | AC010175 | 11 | 12 |
| 7 | AC010784-1 | 13 | 14 |
| 8 | AL049825 | 15 | 16 |
| 9 | AL121585_dal | 17 | 18 |
| 10 | AL121585_da2 | 19 | 20 |
| 11 | AL121585_da3 | 21 | 22 |
| 12 | AL121585 | 23 | 24 |
| 13 | CG54101-06 | 25 | 26 |

PTMAX nucleic acids, and their encoded polypeptides, according to the invention are useful in a variety of applications and contexts. The various PTMAX nucleic acids and polypeptides according to the invention are useful, inter alia, as novel members of the protein families according to the presence of domains and sequence relatedness to previously described proteins.

For example, the PTMA1-6, 9 and 10-13 nucleic acids and their encoded polypeptides include structural motifs that are characteristic of proteins belonging to the prothymosin apha family of proteins. Prothymosin alpha is a thymic hormone that has immunomodulatory, hematopoietic, and anti-neoplastic activities. In particular, prothymosin alpha has the same quantitive and qualitative biological activity as thymosin alpha; i.e., it has efficacy for treatment of immunodeficiency diseases, immunodepressed cancer patients, and for prevention of opportunistic infections in immunosuppressed patients. Thus, PTMA 1-6, 9 and 10-13 nucleic acids and polypeptides, antibodies and related compounds according to the invention will be useful in therapeutic applications implicated in various cancers and immunodeficiency disorders, e.g., AIDS, autoimmune diseases, e.g., lupus erthythematosis and rheumatoid arthritis.

A peptide containing 28 amino acid residues, named thymosin-alpha-1, was originally isolated from calf thymosin fraction 5 and shown to restore various aspects of immune function in several in vitro and in vivo test systems. Thymosin-alpha-1 is one of several hormones or hormone-like substances produced by the thymus gland and derived from a polypeptide precursor. In 1984 Haritos et al. isolated a larger polypeptide precursor containing 113 amino acids from fresh rat thymus named prothymosin-alpha, which contains the thymosin-alpha-1 sequence at its NH2 terminus.

Thymosin-alpha-1 was subsequently isolated from a similar fraction from human thymus and reported to have the same amino acid sequence as bovine thymosin-alpha-1. Prothymosin alpha isolated from human thymus appears to represent the native polypeptide from which thymosin alpha 1, thymosin alpha 11 and other fragments are generated during isolation of thymosin fraction 5. Human prothymosin alpha is a polypeptide of 109 to 114 amino acid residues, and contains the entire thymosin alpha 1 sequence at its amino terminal. The peptide participates in the regulation, differentiation and function of thymic dependent lymphocytes and appears to be at least as potent on a weight basis as thymosin alpha1 in the protection of subject animals against opportunistic infections.

In general, the prothymosin alpha-like proteins of the present invention are thought to have the comparable quantitative and qualitative biological activity as thymosin alpha. An anticipated dosage range is likely to be about 1-100:g/kg/day. Dosages of the nucleic acids of the invention used in gene therapeutic applications are likely to be lower, and administration is likely to be less frequent, than the dosages shown for the proteins.

Human peripheral blood monocytes incubated with prothymosin alpha release thymosin alpha 1 in the culture supernatants. In addition total RNA is found to increase. The production of thymosin alpha 1 involves de novo protein synthesis as shown by the kinetics of its release and the inhibition of its synthesis by actinomycin D and cycloheximide. Thymosin alpha 1 release, possibly in association with HLA-DR, stimulates the proliferation of the T cell population.

Eckert et al. (Int J Immunopharmacol 1997 September-October; 19(9-10):493-500) conducted preclinical studies with prothymosin alpha 1 on mononuclear cells from tumor patients. They studied the immunomodulating potential of the thymic protein, prothymosin alpha1 (Pro alpha1), on the lymphocyte and monocyte directed antitumor reactions of melanoma and colorectal tumors in cancer patients as compared to healthy controls. On average, they found that tumor patients showed lower NK- and LAK-cell activities than healthy controls, being associated with a lower adhesion capacity to tumor target cells. The NK-cell activity of the tumor patients was inversely related to the tumor stage. Pro alpha1 stimulated the impaired patients, LAK-cell activity only at an early stage of disease. The Pro alpha1 effects were associated with an increased adhesion of lymphocytes to tumor target cells and an increased secretion of deficient IFN-gamma and IL-2 secretion. By flow cytometry, Eckert et al. found that pro alpha1 in combination with IL-2 increased the NK-cell markers CD56, CD16/56 and CD25 as well as CD18/11a adhesion molecule expression. Monocytes from tumor patients showed deranged tumoristatic activities compared with healthy controls. Pro alpha1 elevated the mean of the antitumor activity, when applied alone or in combination with rIFN-gamma. In the presence of IFN-gamma, Pro alpha1 stimulated the adhesion of monocytes to cultured tumor cells, mainly by increasing CD54 expression. Pro alpha1 stimulated alone or in combination with IFN-gamma the TNF-alpha and IL-1 beta secretion by monocytes and decreased the high PGE2 and TGF-beta level, especially in the test patient groups.

In addition, prothymosin alpha has been shown to increase the efficacy of anti-viral and chemotheraputic agents. Accordingly, PTMA 1-6, 9 and 10-13 nucleic acids, polypeptides, antibodies and related compounds of the invention may be used to treat viral diseases such as hepatitis C as well as various malignancies. Furthermore, prothymosin alpha has been detected as a product of neoplastically transformed cells. PTMA 1-6, 9 and 10-13 nucleic acids and polypeptides, antibodies and related compounds according to the invention may have therapeutic and diagnostic applications as a diagnostic marker for cancer. Tissue expression analysis as described in EXAMPLE 2 below demonstrates the high expression PTMAX nucleic acids in various cancers,e.g., melanoma, colon and breast, suggesting a potential therapeutic applications of PTMAX nucleic acids and polypeptides either as a diagnostic marker for these cancers or in the treatment of these cancers.

PTMA 7, nucleic acid and encoded polypeptide includes structural motifs that are characteristic of proteins belonging to the oncostatin family of proteins. Oncostatin is an angiostatic CXC cytokine. Angiogenesis is an important normal physiologic process in embryogenesis, wound repair and the female reproductive cycle. However, as a pathological process, it plays a central role in chronic inflammation, fibroproliferative disorders and tumorigenesis. Thus, PTMA 7 nucleic acids and polypeptides, antibodies and related compounds according to the invention will be useful in therapeutic applications implicated in various cancers, coronary artery disease, arthritis, and diabetic retinopathy. In addition, oncostatin had been implicated as an inhibitor of apoptosis. Accordingly, PTMA 7 nucleic acids, polypeptides, antibodies and related compounds of the invention may be used to treat autoimmune diseases, e.g., lupus erthythematosis and rheumatoid arthritis, immune deficiency disorders such as AIDS, and cancers, e.g., melanoma, cervical cancer and Burkitts lymphoma.

PTMA 8, nucleic acids and encoded polypeptides includes structural motifs that are characteristic of proteins belonging to the nerve growth factor family of proteins. Neurotrophins, such as nerve growth factor play an integral role in the growth, differentiation and maintenance of neurons. Thus, PTMA 8 nucleic acids and polypeptides, antibodies and related compounds according to the invention will be useful in therapeutic applications implicated in various neurological diseases, e.g., Parkinson's Disease, Alzheimer's, amyotropic lateral sclerosis and psychiatric disorders. In addition, nerve growth factor has been shown to have a role in neuroimmune interactions. Accordingly, PTMA 8 nucleic acids, polypeptides, antibodies and related compounds of the invention may be used to treat inflammatory disease, e.g., keratoconjunctivitis and asthma, as well as modulate tissue remodeling.

Additional utilities for PTMAX nucleic acids and polypeptides according to the invention are disclosed herein.

1. PTMA-1

A PTMA-1 nucleic acid and polypeptide according to the invention includes the nucleic and encoded polypeptide sequence of clone AC009485_A.

The nucleic acid sequence is 327 nucleotides in length (SEQ ID NO:1), of which nucleotides 1-327 (SEQ ID NO:1) define an open reading frame encoding a polypeptide of 109 amino acids (SEQ ID NO:2).

The AC009485_A nucleic acid has the following sequence:

ATGTCAGATGCAGCTGTAGACACCAGCTCTGAAATCATTGCCAAGGACTTAAAGGAG AAGAAGGAAGTTGTGAAAGAGGCGGAAAATGGAAGAGACGCCCCTGCTAACGGGAA TGCTAATGAGGAAAATGGGGAGCAGGAGGCTGACAAGGAGGTAGATGAAGAAGGGG AAGAAAGTGGGGAGGAAGAGGAGGAGGAAAAAGAAGGTGATGGTGAGGAAGAGGA TGGAGATGAAGAGGAAGCTGAGTCTGCTACAGGCAAGCGGGCAGCTGAAGATGATG AGGATGATGATGTCGATACCAAGAAGCAGAAGACCGACAAGGATGAC (SEQ ID NO:1)

The polypeptide encoded by clone AC009485_A has the following sequence:

MSDAAVDTSSEILAKDLKEKKEVVKEAENGRDAPANGNANEENGEQEADKEVDEEGEES GEEEEEEKEGDGEEEDGDEEEAESATGKRAAEDDEDDDVDTKKQKTDKDD (SEQ ID NO:2)

The calculated molecular weight of PTMA-1 is 11909.9 daltons. Clone AC009485_A was subjected to a search of sequence databases using BLAST programs. It was found, for example, that the amino acid sequence of the invention has 100 of 109 residues (91%), identical to, or 103 of 109 residues (94%) positive to human prothymosin alpha having 109 amino acid residues (accession number ptnr: SPTREMBL-ACC:Q15249PROTHYMOSIN ALPHA(PROT-ALPHA)-HOMO SAPIENS).

Example 2B (discussed below) shows that clone AC009485_A is highly expressed in thymus tissue which is consistent with its identification as a thymic hormone.

2. PTMA-2

A PTMA-2 nucleic acid and polypeptide according to the invention includes the nucleic acid and encoded polypeptide sequence of AC010175_A.0.1.

The nucleic acid sequence is 555 nucleotides in length (SEQ ID NO:3), of which nucleotides 1-342 (SEQ ID NO:3) define an open reading frame encoding a polypeptide of 114 amino acids (SEQ ID NO:4).

The AC010175_A.0.1 nucleic acid has the following sequence:

ATGTCAGACGCAGCCGTAGACACCAGCTCCGAAATCACCACCGAGGACTTAAAGGAG AAGAAGGAAGTTGTGGAAGAGGCGGAAAATGGAAGAGACGCCCCTGCTCACGGGAA TGCTAATGAGGAAAATGGGGAGCCGGAGGCTGACAACGAGGTAGATGAAGAAGAGG AAGAAGGTGGGGAGGAAGAAGGTGATGGTGAGGAAGAGGATGGAGATGAAGATGA GGGAGCTGAGTCAGCTACGGGCAAGCGGGCAGCTGAAGATGATGAGGATAACGATG TCGATACCCAGAAGCAGAAGACCGACGAGGATGACCAGACGGCAAAAAAGGAAAAG TTAAACTAAAAAAAAAGGCCGCCGTGACCTATTCACCCTCCACTTCCCGTCTCAGAAT CTAAACGTGGTCACCTTCGAGTAGAGGGGCCCGCCCGCCCACCGTGGGCAGTGCCAC CCGCAGATGACACGCGCTCTCCACCACCCAACCCAAACCATGAGAATTTGCAACAGG GGAGGGAAAAAGAACCAAAACTTCCAAGGCCCTGCTTTTTTTTTTTT (SEQ ID NO:3)

The polypeptide encoded by clone AC010175_A.0.1 has the following sequence:

MSDAAVDTSSEITTEDLKEKKEVVEEAENGRDAPAHGNANEENGEPEADNEVDEEEEEG GEEEGDGEEEDGDEDEGAESATGKRAAEDDEDNDVDTQKQKTDEDDQTAKKEKLN (SEQ ID NO:4)

The calculated molecular weight of the predicted protein is 12389.2 daltons. Clone AC010175_A.0.1 was subjected to a search of sequence databases using BLAST programs. It was found, for example, that the amino acid sequence of the invention has 112 of 117 residues (95%), identical to, or 113 of 117 residues (96%) positive to human prothymosin alpha pseudogene having 117 amino acid residues (accession number ACC:AAA36485 HUMAN PROTHYMOSIN-ALPHA PSEUDOGENE-HOMO SAPIENS).

3. PTMA-3

A PTMA-3 nucleic acid and polypeptide according to the invention includes the nucleic acid and encoded polypeptide sequence of AC010175_A.9.5.

The nucleic acid sequence is 675 nucleotides in length (SEQ ID NO:5), of which nucleotides 55-397 (SEQ ID NO:5) define an open reading frame encoding a polypeptide of 114 amino acids (SEQ ID NO:6).

The AC010175_A.9.5 nucleic acid has the following sequence:

TGAACTCTCGCTTTCTTTTTAATCCCCTGCATCGGATCACCGGCGTGCCCCACCATGTC AGACGCAGCCGTAGACACCAGCTCCGAAATCACCAACAAGGACTTAAAGGAGAAGA AGGAAGTTGTGGAAGAGGCAGAAAATGGAAGAGACGCCCCTGCTAACGGGAATGCT AATGAGGAAAATGGGGAGCAGGAGGCTGACAATGAGGTAGACGAAGAAGAGGAAG AAGGTGGGGAGGAAGAAGGTGATGGTGAGGAAGAGGATGGAGATGAAGATGAGGA AGCTGAGTCAGCTACGGGCAAGCGGGCAGCTGAAGATGATGAGGATAACGATGTCG ATACCAAGAAGCAGAAGACCGACGAGGATGACCAGACGGCAAAAAAGGAAAAGTTA AACTAAAAAAAAAA AAGGCCGCCGTGACCTATTCACCCTCCACTTCCCGTCTCAGAA TCTAAACGTGGTCACCTTCGAGTAGAGAGGCCCGCCCGCCCACCGTGGGCAGTGCCA CCCGCAGATGACACGCGCTCTCCACCACCCAACCCAAACCATGAGAATTTGCAACAG GGGAGGAAAAAAGAACCAAAACTTCCAAGGCCTGCTTTTTTTCTTAAAAGTACTTTA AAAAGGAAATTTGTTTGTATTTTTTATTTCCATTTTATATTTTTGTACATATTG (SEQ ID NO:5)

The polypeptide encoded by clone AC010175_A.9.5 has the following sequence:

MSDAAVDTSSEITNKDLKEKKEVVEEAENGRDAPANGNANEENGEQEADNEVDEEEEE GGEEEGDGEEEDGDEDEEAESATGKRAAEDDEDNDVDTKKQKTDEDDQTAKKEKLN (SEQ ID NO:6)

The calculated molecular weight of the protein is 12481.4 daltons. Clone AC010175_A.9.5 was subjected to a search of sequence databases using BLAST programs. It was found, for example, that the amino acid sequence of the invention has 106 of 117 residues (90%), identical to, or 110 of 117 residues (94%) positive to human prothymosin alpha pseudogene having 117 amino acid residues (accession number remtrembl-ACC:AAA36485 HUMAN PROTHYMOSIN-ALPHA PSEUDOGENE-HOMO SAPIENS).

4. PTMA-4

A PTMA-4 nucleic acid and polypeptide according to the invention includes the nucleic acid and encoded polypeptide sequence of AC009533_A.

The nucleic acid sequence is 345 nucleotides in length (SEQ ID NO:7), of which nucleotides 1-342 (SEQ ID NO:7) define an open reading frame encoding a polypeptide of 114 amino acids (SEQ ID NO:8).

The AC009533_A nucleic acid has the following sequence:

atgtcagacgcagccgtagacaccagctccgaaatcaccaccgaggacttaaaggagaagaaggaagttgtggaagaggcggaaaatgga agagacgcccctgctcacgggaatgctaatgaggaaaatggggagccggaggctgacaacgaggtagatgaagaagaggaagaaggtgg ggaggaagaaggtgatggtgaggaagaggatggagatgaagatgagggagctgagtcagctaegggcaagcgggcagctgaagatgatg aggatgacgatgtcgatacecagaagcagaagaccgacgaggatgaccagacaecaaaaaaggaaaagttaaactaa (SEQ ID NO:7)

The polypeptide encoded by clone AC009533_A has the following sequence:

MSDAAVDTSSEITTEDLKEKKEVVEEAENGRDAPAHGNANEENGEPEADNEVDEEEEEG GEEEGDGEEEDGDEDEGAESATGKRAAEDDEDDDVDTQKQKTDEDDQTAKKEKLN (SEQ ID NO:8)

The calculated molecular weight of the protein is 12390.2 daltons. Clone AC009533_A was subjected to a search of sequence databases using BLAST programs. It was found, for example, that the nucleic acid sequence has 282 of 322 bases (87%) identical to human prothymosin alpha gene (clone pHG4) (GENBANK-ID:HUMPROC/acc:L21695). It was found, for example, that the amino acid sequence of the invention has 111 of 117 residues (94%), identical to, or 113 of 117 residues (96%) positive to human prothymosin alpha pseudogene (accession number ptnr:REMTREMBL-ACC:G190372); or 99 of 109 residues (90%) identical to, or 102 of 109 residues (93%) positive to a sequence for human prothymosin alpha (accession number ptnr:SPTREMBL-ACC:Q15249). A major distinction of the presently described protein is a deletion of a run of four contiguous glutamate residues after position 63, compared to the related sequences that were identified.

Example 2C (discussed below) shows that clone AC009533_A is highly expressed in thymus tissue which is consistent with its identification as a thymic hormone.

5. PTMA-5

A PTMA-5 nucleic acid and polypeptide according to the invention includes the nucleic acid and encoded polypeptide sequence of AL121585_A.

The nucleic acid sequence is 501 nucleotides in length (SEQ ID NO:9), of which nucleotides 134-460 (SEQ ID NO:9) define an open reading frame encoding a polypeptide of 109 amino acids (SEQ ID NO:10). A PTMA-6 nucleotide sequence according to the invention is also present in clone AL121585_A. The sequences localize to human chromosome 20.

The AL121585_A nucleic acid has the following sequence:

ATTGTTCCTTGTCCGGCTCCTTGCTCGCCGCAGCCGCCTTTACCGCTGCGGACTCCGG ACACTTCATCACCACAGTCCCTGAACTCTCGCTTTCTTTTTAATCCCCTGCATCGGATC ACTGGTGTGCCGGACCATGTCAGACGCAGCCGTAGACACCAGCTCCGAAATCACCAC CAAGGACTTAAAGAAGAAGGAAGCTGTGGAGGAAGCGGAAAATGGAAGAGACACCC CTGCTAATGGGAAGGCTAATGAGGAAAATGGGGAGCAGGAAGCTGACAATGAAGTA GATGAAGAAGAGGAAGAAGGTGGGGAGGAAGACGAGGAGGAAGAAGAAGGCGATG GTGAGGAAGAGGATGGTGATGAAGACGAGGAAGCTGAGTCCGCTACGGTCAAGCGG GCAGCTGAAGATGATGAGAATGATGATGCCTATACCAAGAAGCAGAAGACCAACAA GGATGACTAGACAGCAAAAAAGGAAATGTTAGGAGGGTGACCTATTCA (SEQ ID NO:9)

The polypeptide encoded by clone AL121585_A has the following sequence:

MSDAAVDTSSEITTKDLKKKEAVEEAENGRDTPANGKANEENGEQEADNEVDEEEEEGG EEDEEEEEGDGEEEDGDEDEEAESATVKRAAEDDENDDAYTKKQKTNKDD (SEQ ID NO:10)

The calculated molecular weight of the protein is 12005.8 daltons. Clone AL121585_A was subjected to a search of sequence databases using BLAST programs. It was found, for example, that the nucleotide sequence of the invention has 496 of 501 bases (99%) identical to, or 496 of 501 bases (99%) positive to human prothymosin alpha pseudogene (accession number gb:GENBANK-ID:HUMPROAD/acc:J04800 HUMAN PROTHYMOSIN-ALPHA PSEUDOGENE-HOMO SAPIENS). It was found, for example, that the amino acid sequence of the invention has 99 of 110 residues (90%) identical to, or 103 of 110 residues (93%) positive to human prothymosin alpha (accession number ptnr:PIR-ID:TNHUA PROTHYMOSIN-ALPHA-HUMAN).

6. PTMA-6

A PTMA-6 nucleic acid and polypeptide according to the invention includes the nucleic acid and encoded polypeptide sequence of clone AC010175.

The nucleic acid sequence is 342 nucleotides in length (SEQ ID NO:11), of which nucleotides 1-342 (SEQ ID NO:11) define an open reading frame encoding a polypeptide of 114 amino acids (SEQ ID NO:12).

The AC010175 nucleic acid and encoded polypeptide have the following sequences:

1 ATGTCAGACGCAGCCGTAGACACCAGCTCCGAAATCACCACCGAG

MetSerAspAlaAlaValAspThrSerSerGluIleThrThrGlu

46 GACTTAAAGGAGAAGAAGGAAGTTGTGGAAGAGGCGGAAAATGGA

AspLeuLysGluLysLysGluValValGluGluAlaGluAsnGly

91 AGAGACGCCCCTGCTCACGGGAATGCTAATGAGGAAAATGGGGAG

ArgAspAlaProAlaHisGlyAsnAlaAsnGluGluAsnGlyGlu

136 CCGGAGGCTGACAACGAGGTAGATGAAGAAGAGGAAGAAGGTGGG

ProGluAlaAspAsnGluValAspGluGluGluGluGluGlyGly

181 GAGGAAGAAGGTGATGGTGAGGAAGAGGATGGAGATGAAGATGAG

GluGluGluGlyAspGlyGluGluGluAspGlyAspGluAspGlu

226 GGAGCTGAGTCAGCTACGGGCAAGCGGGCAGCTGAAGATGATGAG

GlyAlaGluSerAlaThrGlyLysArgAlaAlaGluAspAspGlu

271 GATAACGATGTCGATACCCAGAAGCAGAAGACCGACGAGGATGAC

AspAsnAspValAspThrGlnLysGlnLysThrAspGluAspAsp

316 CAGACGGCAAAAAAGGAAAAGTTAAAC (SEQ ID NO:11)

GlnThrAlaLysLysGluLysLeuAsn (SEQ ID NO:12)

The calculated molecular weight of the protein is 12389.2 daltons. Clone AC010175 was subjected to a search of sequence databases using BLAST programs. It was found, for example, that the amino acid sequence of the invention has 98 of 109 residues (89%) identical to, or 102 of 109 residues (93%) positive to human prothymosin alpha a sequence for human prothymosin alpha which is disclosed, for example, in U.S. Pat. Nos. 4,659,694 and 4,716,148.

Example 2A (discussed below) shows that clone AC010175 is highly expressed in thymus tissue which is consistent with its identification as a thymic hormone.

7. PTMA-7

A PTMA-7 nucleic acid and polypeptide according to the invention includes the nucleic acid and encoded polypeptide sequence of clone AC010784-1.

The nucleic acid sequence is 324 nucleotides in length (SEQ ID NO:13), of which nucleotides 1-324 (SEQ ID NO:13) define an open reading frame encoding a polypeptide of 108 amino acids (SEQ ID NO:14).

The AC010784-1 nucleic acid and encoded polypeptide have the following sequences:

1 ATGAGCTCCGCCAGCCGGGTTTTGCGCCTTCAGGCCCCCGGGTTG

MetSerSerAlaSerArgValLeuArgLeuGlnAlaProGlyLeu

46 GTGTTCCTGGGGTTGGTGCTCCTTTCCCTCCCCTCGTCCTCTCTT

ValPheLeuGlyLeuValLeuLeuSerLeuProSerSerSerLeu

91 ACCCTCTCCATTTCCCCCTCAGCTGAAGCTGAAGAAGATGGGGAC

ThrLeuSerIleSerProSerAlaGluAlaGluGluAspGlyAsp

136 CTGCAGTGCCTGTGTGTGAAGACCACCTCCCAGGTCCGTCCCAGG

LeuGlnCysLeuCysValLysThrThrSerGlnValArgProArg

181 CACATCACCAGCCTGGAGGTGATCAAGGCCGGACCCCACTGCCCC

HisIleThrSerLeuGluValIleLysAlaGlyProHisCysPro

226 ACTGCCCAACTGATGGCCACGCTGAAGAATGGAAGGAAAATTTGC

ThrAlaGlnLeuMetAlaThrLeuLysAsnGlyArgLysIeCys

271 TTGGACCTGCAAGCCCCGCTGTACAAGAAAAGGATTAAGAAACTT

LeuAspLeuGInAlaProLeuTyrLysLysArgIleLysLysLeu

316 TTGAAGAGT (SEQ ID NO:13)

LeuLysSer (SEQ ID NO:14)

The calculated molecular weight of the protein is 11680.7 daltons. Clone AC010784-1 was subjected to a search of sequence databases using BLAST programs. It was found, for example, that the amino acid sequence of the invention has 84 of 108 residues (77%) identical to, or 93 of 108 residues (86%) positive to a sequence for platelet factor 4 (PF-4) (oncostatin A). Such related nucleic acids and proteins are disclosed, for example, by Poncz, M., Surrey, S., LaRocco, P., Weiss, M. J., Rappaport, E. F., Conway, T. M. and Schwartz, E. (Blood 69 (1), 219-223 (1987)), and in U.S. Pat. No. 5,656,724.

The novel oncostatin A-like polypeptide of the present invention may serve as a novel growth-modulating factor to which various cells and tissues in the human body respond. The invention is therefore useful in potential therapeutic applications, for a cDNA encoding the oncostatin A-like polypeptide may be useful in gene therapy, and the oncostatin A-like polypeptide may be useful when administered to a subject in need thereof. The novel nucleic acid encoding oncostatin A-like polypeptide, and the polypeptide of the invention, or fragments thereof, may further be useful in diagnostic applications, wherein the presence or amount of the nucleic acid or the polypeptide are to be assessed. These materials are further useful in the generation of antibodies that bind immunospecifically to the novel substances of the invention in therapeutic or diagnostic methods.

8. PTMA-8

A PTMA-8 nucleic acid and polypeptide according to the invention includes the nucleic acid and encoded polypeptide sequence of clone AL049825.

The nucleic acid sequence is 738 nucleotides in length (SEQ ID NO:15), of which nucleotides 13 to 735 (SEQ ID NO:15) define an open reading frame encoding a polypeptide of 241 amino acids (SEQ ID NO:16).

The AL049825 nucleic acid and encoded polypeptide has the following sequence:

1 GTGCATAGCGTAATGTCCATGTTGTTCTACACTCTGATCACAGCT

MetSerMetLeuPheTyrThrLeulleThrAla

46 TTTCTGATCGGCATACAGGCGGAACCACACTCAGAGAGCAATGTC

PheLeuIleGlyIleGlnAlaGluProHisSerGluSerAsnVal

91 CCTGCAGGACACACCATCCCCCAAGCCCACTGGACTAAACTTCAG

ProAlaGlyHisThrIleProGlnAlaHisTrpThrLysLeuGln

136 CATTCCCTTGACACTGCCCTTCGCAGAGCCCGCAGCGCCCCGGCA

HisSerLeuAspThrAlaLeuArgArgAlaArgSerAlaProAla

181 GCGGCGATAGCTGCACGCGTGGCGGGGCAGACCCGCAACATTACT

AlaAlaIleAlaAlaArgValAlaGlyGlnThrArgAsnIleThr

226 GTGGACCCCAGGCTGTTTAAAAAGCGGCGACTCCGTTCACCCCGT

ValAspProArgLeuPheLysLysArgArgLeuArgSerProArg

271 GTGCTGTTTAGCACCCAGCCTCCCCGTGAAGCTGCAGACACTCAG

ValLeuPheSerThrGlnProProArgGluAlaAlaAspThrGln

316 GATCTGGACTTCGAGGTCGGTGGTGCTGCCCCCTTCAACAGGACT

AspLeuAspPheGluValGlyGlyAlaAlaProPheAsnArgThr

361 CACAGGAGCAAGCGGTCATCATCCCATCCCATCTTCCACAGGGGC

HisArgSerLysArgSerSerSerHisProllePheHisArgGly

406 GAATTCTCGGTGTGTGACAGTGTCAGCGTGTGGGTTGGGGATAAG

GluPheSerValCysAspSerValSerValTrpValGlyAspLys

451 ACCACCGCCACAGACATCAAGGGCAAGGAGGTGATGGTGTTGGGA

ThrThrAlaThrAspIleLysGlyLysGluValMetValLeuGly

496 GAGGTGAACATTAACAACAGTGTATTCAAACAGTACTTTTTTGAG

GluValAsnEeAsnAsnSerValPheLysGlnTyrPhePheGlu

541 ACCAAGTGCCGGGACCCAAATCCCGTTGACAGCGGGTGCCGGGGC

ThrLysCysArgAspProAsnProValAspSerGlyCysArgGly

586 ATTGACTCAAAGCACTGGAACTCATATTGTACCACGACTCACACC

IleAspSerLysHisTrpAsnSerTyrCysThrThrThrHisThr

631 TTTGTCAAGGCGCTGACCATGGATGGCAAGCAGGCTGCCTGGCGG

PheValLysAlaLeuThrMetAspGlyLysGlnAlaAlaTrpArg

676 TTTATCCGGATAGATACGGCCTGTGTGTGTGTGCTCAGCAGGAAG

PhelleArgIleAspThrAlaCysValCysValLeuSerArgLys

721 GCTGTGAGAAGAGCCTGA (SEQ ID NO:15)

AlaValArgArgAla (SEQ ID NO:16)

The calculated molecular weight of the protein is 26958.5 daltons. Clone AL049825 was subjected to a search of sequence databases using BLAST programs. It was found, for example, that the amino acid sequence of the invention has 240 of 241 residues (99.5%) similar to a 241 residue sequence for human beta-nerve growth factor precursor (SWISSPROT-ACC:P01138).

This human beta-nerve growth factor precursor-like nucleic acid and polypeptide is also similar to a nucleic acid and polypeptide in PCT publication WO9821234. The protein of this invention includes an alanine at position 35, which differs from the disclosed protein in that a valine appears at this position. According to WO9821234, the prepro region of the polypeptide extends from residue 1 to 121. Thus the variant of the present invention may be implicated in pathological conditions which could arise from inappropriate or incorrect processing. Were this to occur, either the secretion of the protein from one intracellular compartment to another or to the external medium, or the folding of the mature domain of the nerve growth factor beta chain could be adversely affected. Therefore nucleotide sequences and peptide or protein sequences characteristic of the variant of the present invention would find use in diagnostic screening methods, as well as in methods of treating neurological disorders, and in screening for therapeutics that would overcome any pathological state associated with the occurrence of the variant gene and/or its gene product.

9. PTMA-9

APTMA-9 nucleic acid and polypeptide according to the invention includes the nucleic acid and encoded polypeptide sequence of clone AL121585_da1.

The nucleic acid sequence is 345 nucleotides in length (SEQ ID NO:17), of which nucleotides 10-339 (SEQ ID NO:17) define an open reading frame encoding a polypeptide of 110 amino acids (SEQ ID NO:18).

The AL121585_da1 nucleic acid has the following sequence:

TGCCGGACCATGTCAGACGCAGCCGTAGACACCAGCTCCGAAATCACCACCAAGGAC TTAAAGGAGAAGAAGGAAGTTGTGGAAGAGGCAGAAAATGGAAGAGACGCCCCTGC TAACGGGAATGCTAATGAGGAAAATGGGGAGCAGGAGGCTGACAATGAGGTAGACG AAGAAGAGGAAGAAGGTGGGGAGGAAGAGGAGGAGGAAGAAGAAGGTGATGGTGA GGAAGAGGATGGAGATGAAGATGAGGAAGCTGAGTCAGCTACGGGCAAGCGGGCAG CTGAAGATGATGAGGATGACGATGTCGATACCAAGAAGCAGAAGACCAACAAGGAT GACTAGACA (SEQ ID NO:17).

The AL121585_da1 polypeptide has the following sequence (using the one-letter amino acid code):

MSDAAVDTSSEITTKDLKEKKEVVEEAENGRDAPANGNANEENGEQEADNEVDE EEEEGGEEEEEEEEGDGEEEDGDEDEEAESATGKRAAEDDEDDDVDTKKQKTNKDD (SEQ ID NO:18).

The calculated molecular weight of the protein is 12071.8 daltons. Clone AC010175 was subjected to a search of sequence databases using BLAST programs. It was found, for example, that the amino acid sequence of the invention has 108 of 110 residues (98%) identical to, or all 110 residues (100%) positive to human prothymosin alpha (PIR ID:TNHUA).

10. PTMA-10

A PTMA-10 nucleic acid and polypeptide according to the invention includes the nucleic acid and encoded polypeptide sequence of clone AL121585_da2.

The nucleic acid sequence is 350 nucleotides in length (SEQ ID NO:19), of which nucleotides 10-348 (SEQ ID NO:19) define an open reading frame encoding a polypeptide of 113 amino acids (SEQ ID NO:20).

The AL121585_da2 nucleic acid has the following sequence:

TGCCGGACCATGTCAGACGCAGCCGTACACACCACCTCCGAAATCACCACCAAGGAC TTAAAGGAGAAGAAGGAAGTTGTGGAAGAGGCAGAAAATGGAAGAGACGCCCCTGC TAACGGGAATGCTAATGAGGAAAATGGGGAGCAGGAGGCTGACAATGAGGTAGACC AAGAAGAGGAAGAAGGTGGGGAGGAAGAGGAGGAGGAAGAAGAAGGTGATGGTGA GGAAGAGGATGGAGATGAAGATGAGGAAGCTGAGTCACCTACGGGCAACCGGGCAG CTGAAGATGATGAGGATGACGATGTCAATACCAAGGAAGGCGGAAGGACCAACCAA GGGATGACTAGACA (SEQ ID NO:19).

The AL121585_da2 polypeptide has the following sequence (using the one-letter amino acid code):

MSDAAVHTTSEITTKDLKEKKEVVEEAENGRDAPANGNANEENGEQEADNEVDQEEEE GGEEEEEEEEGDGEEEDGDEDEEAESPTGNRAAEDDEDDDVNTKEGGRTNQGMTR (SEQ ID NO:20)

The calculated molecular weight of the protein is 12348.2 daltons. Clone AL121585_da2 was subjected to a search of sequence databases using BLAST programs. It was found, for example, that the amino acid sequence of the invention has 96 of 103 residues (93%) identical to, or 100 of 1039 residues (97%) positive to a 110 residue human prothymosin alpha (PIR ID:TNHUA).

11. PTMA-11

A variant of PTMA-5 given by the nucleic acid and its encoded polypeptide designated AL121585_da3 is also presented. The nucleic acid sequence is 497 nucleotides in length (SEQ ID NO:21), of which nucleotides 134-463 define an open reading frame encoding a polypeptide of 110 amino acids (SEQ ID NO:22). BlastN analysis showed 95% identity to nucleotide sequence of PROTHYA-5 (474/497) and 95% positives (474/497).

AL121585_da3 nucleic acid has the following sequence:

ATTGTTCCTTGTCCGGCTCCTTGCTCGCCGCAGCCGCCTTTACCGCTGCGGACTCCGG ACACTTCATCACCACAGTCCCTGAACTCTCGCTTTCTTTTTAATCCCCTGCATCGGATC ACTGGTGTGCCGGACCATGTCAGACGCAGCCGTAGACACCAGCTCCGAAATCACCAC CAAGGACTTAAAGGAGAAGAAGGAAGTTGTGGAAGAGGCAGAAAATGGAAGAGAC GCCCCTGCTAACGGGAATGCTAATGAGGAAAATGGGGAGCAGGAGGCTGACAATGA GGTAGACGAAGAAGAGGAAGAAGGTGGGGAGGAAGAGGAGGAGGAAGAAGAAGGT GATGGTGAGGAAGAGGATGGAGATGAAGATGAGGAAGCTGAGTCAGCTACGGGCAA GCGGGCAGCTGAAGATGATGAGGATGACGATGTCGATACCAAGAAGCAGAAGACCA ACAAGGATGACTAGACAGCAAAAAAGGAAATGTTAGGAGGGTGAC (SEQ ID NO:21)

The polypeptide encoded by clone AL121585_da3 has the following sequence:

MSDAAVDTSSEITTKDLKEKKEVVEEAENGRDAPANGNANEENGEQEADNEVDEEEEEG GEEEEEEEEGDGEEEDGDEDEEAESATGKRAAEDDEDDDVDTKKQKTNKDD (SEQ ID NO:22).

The calculated molecular weight of the protein of SEQ ID NO:22 is 12071.8. It was found from the BlastP program that the amino acid sequence of the invention has 108 of 110 residues (98%) identical to, or 100% positive to human prothymosin alpha having 110 amino acid residues (accession number ptnr:PIR-ID:TNHUA PROTHYMOSIN-ALPHA-HUMAN).

12. PTMA-12

A PTMA-1 nucleic acid and polypeptide according to the invention includes the nucleic acid and encoded polypeptide sequence of clone AL121585.

The nucleic acid sequence is 497 nucleotides in length (SEQ ID NO:23), of which nucleotides 134-460 (SEQ ID NO:23) define an open reading frame encoding a polypeptide of 110 amino acids (SEQ ID NO:24).

The AL121585 nucleic acid has the following sequence:

ATTGTTCCTTGTCCGGCTCCTTGCTCGCCGCAGCCGCCTTTACCGCTGCGGACTCCGG ACACTTCATCACCACAGTCCCTGAACTCTCGCTTTCTTTTTAATCCCCTGCATCGGATC ACTGGTGTGCCGGACCATGTCAGACGCAGCCGTAGACACCAGCTCCGAAATCACCAC CAAGGACTTAAAGGAGAAGAAGGAAGTTGTGGAAGAGGCAGAAAATGGAAGAGAC GCCCCTGCTAACGGGAATGCTAATGAGGAAAATGGGGAGCAGGAGGCTGACAATGA GGTAGACGAAGAAGAGGAAGAAGGTGGGGAGGAAGAGGAGGAGGAAGAAGAAGGT GATGGTGAGGAAGAGGATGGAGATGAAGATGAGGAAGCTGAGTCAGCTACGGGCAA GCGGGCAGCTGAAGATGATGAGGATGACGATGTCGATACCAAGAAGCAGAAGACCA ACAAGGATGACTAGACAGCAAAAAAGGAAATGTTAGGAGGGTGAC (SEQ ID NO:23).

The AL121585 polypeptide has the following sequence (using the one-letter amino acid code):

MSDAAVDTSSEITTKDLKEKKEVVEEAENGRDAPANGNANEENGEQEADNEVDEEEEEG GEEEEEEEEGDGEEEDGDEDEEAESATGKRAAEDDEDDDVDTKKQKTNKDD (SEQ ID NO:24)

Clone AL121585 was subjected to a search of sequence databases using BLAST programs. It was found, for example, that the nucleic acid sequence of this invention has 459 of 486 bases (94%) identical to a Homo sapien prothymosin alpha mRNA (GENBANK-ID: M14483) and that the amino acid sequence of the invention has 108 of 110 residues (98%) identical to, or 110 of 110 residues (100%) similar to a 110 residue Homo sapien prothymosin alpha protein (PIR ID:TNHUA). Clone AL121585 disclosed in this invention maps to chromosome 20 and is expressed in at least some of the following tissues: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. In addition, Clone AL121585 is predicted to be expressed in the following tissues because of the expression pattern of Human prothymosin alpha (GENBANK-ID:M14483) closely related prothymosin alpha homolog in species Homo sapiens: spleen.

The following positions in clone AL121585 disclosed in the invention have been identified as having the following variations (SNPs):

| Consensus | | Base | |
|---|---|---|---|
| Position | Depth | Change | PAF |
| 124 | 21 | T > G | 0.476 |
| 188 | 21 | G > — | 0.286 |
| 189 | 21 | A > — | 0.143 |
| 190 | 21 | A > — | 0.429 |
| 191 | 21 | G > — | 0.429 |
| 202 | 20 | T > C | 0.450 |
| 209 | 20 | A > G | 0.450 |
| 212 | 20 | G > A | 0.450 |
| 215 | 20 | A > G | 0.450 |
| 231 | 20 | G > A | 0.450 |
| 242 | 20 | C > T | 0.450 |
| 248 | 20 | T > G | 0.450 |
| 275 | 20 | G > A | 0.450 |
| 287 | 20 | G > A | 0.450 |
| 293 | 20 | C > T | 0.450 |
| 323 | 20 | G > C | 0.450 |
| 341 | 20 | T > C | 0.450 |
| 362 | 20 | A > T | 0.450 |
| 371 | 20 | T > C | 0.450 |
| 386 | 21 | A > C | 0.450 |
| 391 | 21 | C > — | 0.381 |

13. PTMA-13

A PTMA-13 nucleic acid and polypeptide according to the invention includes the nucleic acid and encoded polypeptide sequence of clone CG54101-06.

The nucleic acid sequence is 493 nucleotides in length (SEQ ID NO:25), of which nucleotides 134-481 (SEQ ID NO:25) define an open reading frame encoding a polypeptide of 116 amino acids (SEQ ID NO:26).

The CG54101-06 nucleic acid has the following sequence:

ATTGTTCCTTGTCCGGCTCCTTGCTCGCCGCAGCCGCCTTTACCGCTGCGGACTCCGG ACACTTCATCACCACAGTCCCTGAACTCTCGCTTTCTTTTTACTCCCCTGCATCGGATC ACTGGTGTGCCGGACCATGTCAGACGCAGCCGTAGACACCAGCTCCGAAATCACCAC CAAGGACTTAGAGAAGAAGGAAGCTGTGGAGGAAGCGGAAAATGGAAGAGACACCC CTGCTAATGGGAAGGCTAATGAGGAAAATGGGGAGCAGGAAGCTGACAATGAAGTA GATGAAGAAGAGGAAGAAGGTGGGGAGGAAGACGAGGAGGAAGAAGAACGCGATG GTGAGGAAGAGGATGGTGATGAAGACGAGGAAGCTGAGTCCGCTAGGTCAAGCGGG CAGCTGATGATGATGAGAATGATGATGCCTATACCAAGAAGCAGAAGACCAACAAG GATGACTAGACAGCAAAAAGGAAATGTTAGGAGGGTGAC (SEQ ID NO:25).

The CG54101-06 polypeptide has the following sequence (using the one-letter amino acid code):

MSDAAVDTSSEITTKDLEKKEAVEEAENGRDTPANGKANEENGEQEADNEVDEEEEEGG EEDEEEEERDGEEEDGDEDEEAESARSSGQLMMMRMMMPIPRSRRPTRMTRQQKRKC (SEQ ID NO:26)

Clone CG54101-06 was subjected to a search of sequence databases using BLAST programs. It was found, for example, that the nucleic acid sequence of this invention has 489 of 494 bases (98%) identical to a gb:GENBANK-ID:AX111668|acc:AX111668.1 mRNA from Homo sapiens (Sequence 9 from Patent WO0123572) and that the full amino acid sequence of the protein of the invention was found to have 113 of 116 amino acid residues (97%) identical to, and 114 of 116 amino acid residues (98%) similar to, the 116 amino acid residue ptnr:SPTREMBL-ACC:Q9NQ22 protein from Homo sapiens (BA504H3.2 SIMILAR TO PTMA (PROTHYMOSIN-ALPHA)). Clone CG54101-06 disclosed in this invention maps to chromosome 20 and is expressed in at least some of the following tissues: aorta.

### PTMAX Nucleic Acids

One aspect of the invention pertains to isolated nucleic acid molecules that encode PTMAX polypeptides or biologically active portions thereof. Also included in the invention are nucleic acid fragments sufficient for use as hybridization probes to identify PTMAX-encoding nucleic acids (e.g., PTMAX mRNA) and fragments for use as PCR primers for the amplification or mutation of PTMAX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule can be single-stranded or double-stranded, but preferably is double-stranded DNA.

“Probes” refer to nucleic acid sequences of variable length, preferably between at least about 10 nucleotides (nt), 100 nt, or as many as about, e.g., 6,000 nt, depending on use. Probes are used in the detection of identical, similar, or complementary nucleic acid sequences. Longer length probes are usually obtained from a natural or recombinant source, are highly specific and much slower to hybridize than oligomers. Probes may be single- or double-stranded and designed to have specificity in PCR, membrane-based hybridization technologies, or ELISA-like technologies.

An “isolated” nucleic acid molecule is one that is separated from other nucleic acid molecules which are present in the natural source of the nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated PTMAX nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the present invention, e.g., a nucleic acid molecule having the nucleotide sequence of SEQ ID NO:1, 3, 5, 7, 9, 11, 13, 15, 17, 19, 21, 23, 25 or 51, or a complement of any of these nucleotide sequences, can be isolated using standard molecular biology techniques and the sequence information provided herein. Using all or a portion of the nucleic acid sequence of SEQ ID NO:1, 3, 5, 7, 9, 11, 13, 15, 17, 19, 21, 23, 25 or 51 as a hybridization probe, PTMAX molecules can be isolated using standard hybridization and cloning techniques (e.g., as described in Sambrook et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to PTMAX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment, an oligonucleotide comprising a nucleic acid molecule less than 100 nt in length would further comprise at lease 6 contiguous nucleotides of SEQ ID NO:1, 3, 5, 7, 9, 11, 13, 15, 17, 19, 21, 23, 25 or 51, or a complement thereof. Oligonucleotides maybe chemically synthesized and may be used as probes.

In another embodiment, an isolated nucleic acid molecule of the invention comprises a nucleic acid molecule that is a complement of the nucleotide sequence shown in SEQ ID NO:1, 3, 5, 7, 9, 11, 13, 15, 17, 19, 21, 23, 25 or 51, or a portion of this nucleotide sequence, e.g., a fragment that can be used as a probe or primer or a fragment encoding a biologically active portion of PTMAX. A nucleic acid molecule that is complementary to the nucleotide sequence shown in SEQ ID NO:1, 3, 5, 7, 9, 11, 13, 15, 17, 19, 21, 23, 25 or 51 is one that is sufficiently complementary to the nucleotide sequence shown in SEQ ID NO:1, 3, 5, 7, 9, 11, 13, 15, 17, 19, 21, 23, 25 or 51 that it can hydrogen bond with little or no mismatches to the nucleotide sequence shown in SEQ ID NO:1, 3, 5, 7, 9, 11, 13, 15, 17, 19, 21, 23, 25 or 51, thereby forming a stable duplex.

As used herein, the term “complementary” refers to Watson-Crick or Hoogsteen base pairing between nucleotide units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, etc. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 30%, 50%, 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of PTMAX polypeptide. Isoforms can be expressed in different tissues of the same organism as a result of, for example, alternative splicing of RNA. Alternatively, isoforms can be encoded by different genes. In the present invention, homologous nucleotide sequences include nucleotide sequences encoding for a PTMAX polypeptide of species other than humans, including, but not limited to, mammals, and thus can include, e.g., mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the nucleotide sequence encoding human PTMAX protein. Homologous nucleic acid sequences include those nucleic acid sequences that encode conservative amino acid substitutions (see below) in SEQ ID NO:1, 3, 5, 7, 9, 11, 13, 15, 17, 19, 21, 23, 25 or 51 as well as a polypeptide having PTMAX activity. Biological activities of the PTMAX proteins are described below. A homologous amino acid sequence does not encode the amino acid sequence of a human PTMAX polypeptide.

A PTMAX polypeptide is encoded by the open reading frame (“ORF”) of a PTMAX nucleic acid. The invention includes the nucleic acid sequence comprising the stretch of nucleic acid sequences of SEQ ID NO:1, 3, 5, 7, 9, 11, 13, 15, 17, 19, 21, 23, 25 or 51 that comprises the ORF of that nucleic acid sequence and encodes a polypeptide of SEQ ID NO:2, 4, 6, 8, 10, 12, 14, 16, 18, 20, 22, 24, 26 or 52.

An “open reading frame” (“ORF”) corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bona fide cellular protein, a minimum size requirement is often set, for example, a stretch of DNA that would encode a protein of 50 amino acids or more.

The nucleotide sequence determined from the cloning of the human PTMAX gene allows for the generation of probes and primers designed for use in identifying and/or cloning PTMAX homologues in other cell types, e.g. from other tissues, as well as PTMAX homologues from other mammals. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence of SEQ ID NO:1, 3, 5, 7, 9, 11, 13, 15, 17, 19, 21, 23, 25 or 51, or an anti-sense strand nucleotide sequence of SEQ ID NO:1, 3, 5, 7, 9, 11, 13, 15, 17, 19, 21, 23, 25 or 51, or of a naturally occurring mutant of SEQ ID NO:1, 3, 5, 7, 9, 11, 13, 15, 17, 19, 21, 23, 25 or 51.

Probes based on the human PTMAX nucleotide sequence can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissue which misexpress a PTMAX protein, such as by measuring a level of a PTMAX-encoding nucleic acid in a sample of cells from a subject e.g., detecting PTMAX mRNA levels or determining whether a genomic PTMAX gene has been mutated or deleted.

“A polypeptide having a biologically active portion of PTMAX” refers to polypeptides exhibiting activity similar, but not necessarily identical to, an activity of a polypeptide of the present invention, including mature forms, as measured in a particular biological assay, with or without dose dependency. A nucleic acid fragment encoding a “biologically active portion of PTMAX” can be prepared by isolating a portion of SEQ ID NO:1, 3, 5, 7, 9, 11, 13, 15, 17, 19, 21, 23, 25 or 51 that encodes a polypeptide having a PTMAX biological activity (the biological activities of the PTMAX proteins are described below), expressing the encoded portion of PTMAX protein (e.g., by recombinant expression in vitro) and assessing the activity of the encoded portion of PTMAX.

### PTMAX Variants

The invention further encompasses nucleic acid molecules that differ from the nucleotide sequence shown in SEQ ID NO:1, 3, 5, 7, 9, 11, 13, 15, 17, 19, 21, 23, 25 or 51 due to degeneracy of the genetic code and thus encode the same PTMAX protein as that encoded by the nucleotide sequence shown in SEQ ID NO:1, 3, 5, 7, 9, 11, 13, 15, 17, 19, 21, 23, 25 or 51. In another embodiment, an isolated nucleic acid molecule of the invention has a nucleotide sequence encoding a protein having an amino acid sequence shown in SEQ ID NO:2, 4, 6, 8, 10, 12, 14, 16, 18, 20, 22, 24, 26 or 52.

In addition to the human PTMAX nucleotide sequence shown in SEQ ID NO:1, 3, 5, 7, 9, 11, 13, 15, 17, 19, 21, 23, 25 or 51, it will be appreciated by those skilled in the art that DNA sequence polymorphisms that lead to changes in the amino acid sequences of PTMAX may exist within a population (e.g., the human population). Such genetic polymorphism in the PTMAX gene may exist among individuals within a population due to natural allelic variation. As used herein, the terms “gene” and “recombinant gene” refer to nucleic acid molecules comprising an open reading frame encoding an PTMAX protein, preferably a mammalian PTMAX protein. Such natural allelic variations can typically result in 1-5% variance in the nucleotide sequence of the PTMAX gene. Any and all such nucleotide variations and resulting amino acid polymorphisms in PTMAX that are the result of natural allelic variation and that do not alter the functional activity of PTMAX are intended to be within the scope of the invention.

Moreover, nucleic acid molecules encoding PTMAX proteins from other species, and thus that have a nucleotide sequence that differs from the human sequence of SEQ ID NO:1, 3, 5, 7, 9, 11, 13, 15, 17, 19, 21, 23, 25 or 51 are intended to be within the scope of the invention. Nucleic acid molecules corresponding to natural allelic variants and homologues of the PTMAX cDNAs of the invention can be isolated based on their homology to the human PTMAX nucleic acids disclosed herein using the human cDNAs, or a portion thereof, as a hybridization probe according to standard hybridization techniques under stringent hybridization conditions. For example, a soluble human PTMAX cDNA can be isolated based on its homology to human membrane-bound PTMAX. Likewise, a membrane-bound human PTMAX cDNA can be isolated based on its homology to soluble human PTMAX.

Accordingly, in another embodiment, an isolated nucleic acid molecule of the invention is at least 6 nucleotides in length and hybridizes under stringent conditions to the nucleic acid molecule comprising the nucleotide sequence of SEQ ID NO:1, 3, 5, 7, 9, 11, 13, 15, 17, 19, 21, 23, 25 or 51. In another embodiment, the nucleic acid is at least 10, 25, 50, 100, 250, 500, 750, 1000, 1500, or 2000 or more nucleotides in length. In another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding PTMAX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to the sequence of SEQ ID NO:1, 3, 5, 7, 9, 11, 13, 15, 17, 19, 21, 23, 25 or 51 corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NO:1, 3, 5, 7, 9, 11, 13, 15, 17, 19, 21, 23, 25 or 51 or fragments, analogs or derivatives thereof, under conditions of moderate stringency is provided. A non-limiting example of moderate stringency hybridization conditions are hybridization in 6×SSC, 5×Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known in the art. See, e.g., Ausubel et al (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY.

In a third embodiment, a nucleic acid that is hybridizable to the nucleic acid molecule comprising the nucleotide sequence of SEQ ID NO:1, 3, 5, 7, 9, 11, 13, 15, 17, 19, 21, 23, 25 or 51 or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981, Proc Natl Acad Sci USA 78: 6789-6792.

### Conservative Mutations

In addition to naturally-occurring allelic variants of a PTMAX sequence that may exist in the population, the skilled artisan will further appreciate that changes can be introduced by mutation into the nucleotide sequence of SEQ ID NO:1, 3, 5, 7, 9, 11, 13, 15, 17, 19, 21, 23, 25 or 51, thereby leading to changes in the amino acid sequence of the encoded PTMAX protein, without altering the functional ability of the PTMAX protein. For example, nucleotide substitutions leading to amino acid substitutions at “non-essential” amino acid residues can be made in the sequence of SEQ ID NO:1, 3, 5, 7, 9, 11, 13, 15, 17, 19, 21, 23, 25 or 51. A “non-essential” amino acid residue is a residue that can be altered from the wild-type sequence of PTMAX without altering the biological activity, whereas an “essential” amino acid residue is required for biological activity. For example, amino acid residues that are conserved among the PTMAX proteins of the present invention, are predicted to be particularly unamenable to alteration. Amino acids for which conservative substitutions can be made are known in the art.

Another aspect of the invention pertains to nucleic acid molecules encoding PTMAX proteins that contain changes in amino acid residues that are not essential for activity. Such PTMAX proteins differ in amino acid sequence from SEQ ID NO:1, 3, 5, 7, 9, 11, 13, 15, 17, 19, 21, 23, 25 or 51, yet retain biological activity. In one embodiment, the isolated nucleic acid molecule comprises a nucleotide sequence encoding a protein, wherein the protein comprises an amino acid sequence at least about 45% homologous to the amino acid sequence of SEQ ID NO:2, 4, 6, 8, 10, 12, 14, 16, 18, 20, 22, 24, 26 or 52. Preferably, the protein encoded by the nucleic acid molecule is at least about 60% homologous to SEQ ID NO:2, 4, 6, 8, 10, 12, 14, 16, 18, 20, 22, 24, 26 or 52, more preferably at least about 70% homologous to SEQ ID NO:2, 4, 6, 8, 10, 12, 14, 16, 18, 20, 22, 24, 26 or 52, still more preferably at least about 80% homologous to SEQ ID NO:2, 4, 6, 8, 10, 12, 14, 16, 18, 20, 22, 24, 26 or 52, even more preferably at least about 90% homologous to SEQ ID NO:2, 4,6,8, 10, 12, 14, 16, 18, 20, 22, 24, 26 or 52 and most preferably at least about 95% homologous to SEQ ID NO:2, 4, 6, 8, 10, 12, 14, 16, 18, 20, 22, 24, 26 or 52.

An isolated nucleic acid molecule encoding an PTMAX protein homologous to the protein of SEQ ID NO:2, 4, 6, 8, 10, 12, 14, 16, 18, 20, 22, 24, 26 or 52 can be created by introducing one or more nucleotide substitutions, additions or deletions into the nucleotide sequence of SEQ ID NO:2, 4, 6, 8, 10, 12, 14, 16, 18, 20, 22, 24, 26 or 52 such that one or more amino acid substitutions, additions or deletions are introduced into the encoded protein.

Mutations can be introduced into SEQ ID NO:2, 4, 6, 8, 10, 12, 14, 16, 18, 20, 22, 24, 26 or 52 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in PTMAX is replaced with another amino acid residue from the same side chain family. Alternatively, in another embodiment, mutations can be introduced randomly along all or part of an PTMAX coding sequence, such as by saturation mutagenesis, and the resultant mutants can be screened for PTMAX biological activity to identify mutants that retain activity. Following mutagenesis of SEQ ID NO:2, 4, 6, 8, 10, 12, 14, 16, 18, 20, 22, 24, 26 or 52, the encoded protein can be expressed by any recombinant technology known in the art and the activity of the protein can be determined.

In one embodiment, a mutant PTMAX protein can be assayed for (1) the ability to form protein:protein interactions with other PTMAX proteins, other cell-surface proteins, or biologically active portions thereof, (2) complex formation between a mutant PTMAX protein and an PTMAX ligand; (3) the ability of a mutant PTMAX protein to bind to an intracellular target protein or biologically active portion thereof; (e.g. avidin proteins).

### Antisense

Another aspect of the invention pertains to isolated antisense nucleic acid molecules that are hybridizable to or complementary to the nucleic acid molecule comprising the nucleotide sequence of SEQ ID NO:1, 3, 5, 7, 9, 11, 13, 15, 17, 19, 21, 23, 25 or 51, or fragments, analogs or derivatives thereof. An “antisense” nucleic acid comprises a nucleotide sequence that is complementary to a “sense” nucleic acid encoding a protein, e.g., complementary to the coding strand of a double-stranded cDNA molecule or complementary to an mRNA sequence. In specific aspects, antisense nucleic acid molecules are provided that comprise a sequence complementary to at least about 10, 25, 50, 100, 250 or 500 nucleotides or an entire PTMAX coding strand, or to only a portion thereof. Nucleic acid molecules encoding fragments, homologs, derivatives and analogs of an PTMAX protein of SEQ ID NO:2, 4, 6, 8, 10, 12, 14, 16, 18, 20, 22, 24, 26 or 52, or antisense nucleic acids complementary to an PTMAX nucleic acid sequence of SEQ ID NO:1, 3, 5, 7, 9, 11, 13, 15, 17, 19, 21, 23, 25 or 51, are additionally provided.

In one embodiment, an antisense nucleic acid molecule is antisense to a “coding region” of the coding strand of a nucleotide sequence encoding PTMAX. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding PTMAX. The term “noncoding region” refers to 5′ and 3′ sequences which flank the coding region that are not translated into amino acids (i.e., also referred to as 5′ and 3′ untranslated regions).

Given the coding strand sequences encoding PTMAX disclosed herein antisense nucleic acids of the invention can be designed according to the rules of Watson and Crick or Hoogsteen base pairing. The antisense nucleic acid molecule can be complementary to the entire coding region of PTMAX mRNA, but more preferably is an oligonucleotide that is antisense to only a portion of the coding or noncoding region of PTMAX mRNA. For example, the antisense oligonucleotide can be complementary to the region surrounding the translation start site of PTMAX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used.

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an PTMAX protein to thereby inhibit expression of the protein, e.g., by inhibiting transcription and/or translation. The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids Res 15: 6625-6641). The antisense nucleic acid molecule can also comprise a 2′-o-methylribonucleotide (Inoue et al. (1987) Nucleic Acids Res 15: 6131-6148) or a chimeric RNA-DNA analogue (Inoue et al. (1 987) FEBS Lett 215: 327-330).

### Ribozymes and PNA Moieties

Nucleic acid modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave PTMAX mRNA transcripts to thereby inhibit translation of PTMAX mRNA. A ribozyme having specificity for an PTMAX-encoding nucleic acid can be designed based upon the nucleotide sequence of an PTMAX cDNA disclosed herein (i.e., SEQ ID NO:1, 3, 5, 7, 9, 11, 13, 15, 17, 19, 21, 23, 25 or 51). For example, a derivative of a Tetrahymena L-19 IVS RNA can be constructed in which the nucleotide sequence of the active site is complementary to the nucleotide sequence to be cleaved in an PTMAX-encoding mRNA. See, e.g., Cech et al. U.S. Pat. No. 4,987,071; and Cech et al. U.S. Pat. No. 5,116,742. Alternatively, PTMAX mRNA can be used to select a catalytic RNA having a specific ribonuclease activity from a pool of RNA molecules. See, e.g., Bartel et al., (1993) Science 261:1411-1418.

Alternatively, PTMAX gene expression can be inhibited by targeting nucleotide sequences complementary to the regulatory region of the PTMAX (e.g., the PTMAX promoter and/or enhancers) to form triple helical structures that prevent transcription of the PTMAX gene in target cells. See generally, Helene. (1991) Anticancer Drug Des. 6: 569-84; Helene. et al. (1992) Ann. N.Y. Acad. Sci. 660:27-36; and Maher (1992) Bioassays 14: 807-15.

In various embodiments, the nucleic acids of PTMAX can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorg Med Chem 4: 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996) above; Perry-O'Keefe et al. (1996) PNAS 93: 14670-675.

PNAs of PTMAX can be used in therapeutic and diagnostic applications. For example, PNAs can be used as antisense or antigene agents for sequence-specific modulation of gene expression by, e.g., inducing transcription or translation arrest or inhibiting replication. PNAs of PTMAX can also be used, e.g., in the analysis of single base pair mutations in a gene by, e.g., PNA directed PCR clamping; as artificial restriction enzymes when used in combination with other enzymes, e.g., S1 nucleases (Hyrup B. (1996) above); or as probes or primers for DNA sequence and hybridization (Hyrup et al. (1996), above; Perry-O'Keefe (1996), above).

In another embodiment, PNAs of PTMAX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of PTMAX can be generated that may combine the advantageous properties of PNA and DNA. Such chimeras allow DNA recognition enzymes, e.g., RNase H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup (1996) above). The synthesis of PNA-DNA chimeras can be performed as described in Hyrup (1996) above and Finn et al. (1996) Nucl Acids Res 24: 3357-63. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA (Mag et al. (1989) Nucl Acid Res 17: 5973-88). PNA monomers are then coupled in a stepwise manner to produce a chimeric molecule with a 5′ PNA segment and a 3′ DNA segment (Finn et al. (1996) above). Alternatively, chimeric molecules can be synthesized with a 5′ DNA segment and a 3′ PNA segment. See, Petersen et al. (1975) Bioorg Med Chem Lett 5: 1119-11124.

In other embodiments, the oligonucleotide may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO88/09810) or the blood-brain barrier (see, e.g., PCT Publication No. WO89/10134). In addition, oligonucleotides can be modified with hybridization triggered cleavage agents (See, e.g., Krol et al., 1988, BioTechniques 6:958-976) or intercalating agents. (See, e.g., Zon, 1988, Pharm. Res. 5: 539-549). To this end, the oligonucleotide may be conjugated to another molecule, e.g., a peptide, a hybridization triggered cross-linking agent, a transport agent, a hybridization-triggered cleavage agent, etc.

### PTMAX Polypeptides

A polypeptide according to the invention includes a polypeptide including the amino acid sequence of PTMAX polypeptides whose sequences are provided by SEQ ID NO:2, 4, 6, 8, 10, 12, 14, 16, 18, 20, 22, 24, 26 or 52. The invention also includes a mutant or variant protein any of whose residues may be changed from the corresponding residues shown in SEQ ID NO:2, 4, 6, 8, 10, 12, 14, 16, 18, 20, 22, 24, 26 or 52 while still encoding a protein that maintains its PTMAX activities and physiological functions, or a functional fragment thereof. In the mutant or variant protein, up to 20% or more of the residues may be so changed.

In general, a PTMAX variant that preserves PTMAX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated PTMAX proteins, and biologically active portions thereof, or derivatives, fragments, analogs or homologs thereof. Also provided are polypeptide fragments suitable for use as immunogens to raise anti-PTMAX antibodies. In one embodiment, native PTMAX proteins can be isolated from cells or tissue sources by an appropriate purification scheme using standard protein purification techniques. In another embodiment, PTMAX proteins are produced by recombinant DNA techniques. Alternative to recombinant expression, a PTMAX protein or polypeptide can be synthesized chemically using standard peptide synthesis techniques.

An “isolated” or “purified” polypeptide or protein or biologically active portion thereof is substantially free of cellular material or other contaminating proteins from the cell or tissue source from which the PTMAX protein is derived, or substantially free from chemical precursors or other chemicals when chemically synthesized. The language “substantially free of cellular material” includes preparations of PTMAX protein in which the protein is separated from cellular components of the cells from which it is isolated or recombinantly produced. In one embodiment, the language “substantially free of cellular material” includes preparations of PTMAX protein having less than about 30% (by dry weight) of non-PTMAX protein (also referred to herein as a “contaminating protein”), more preferably less than about 20% of non-PTMAX protein, still more preferably less than about 10% of non-PTMAX protein, and most preferably less than about 5% non-PTMAX protein. When the PTMAX protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of PTMAX protein in which the protein is separated from chemical precursors or other chemicals that are involved in the synthesis of the protein. In one embodiment, the language “substantially free of chemical precursors or other chemicals” includes preparations of PTMAX protein having less than about 30% (by dry weight) of chemical precursors or non-PTMAX chemicals, more preferably less than about 20% chemical precursors or non-PTMAX chemicals, still more preferably less than about 10% chemical precursors or non-PTMAX chemicals, and most preferably less than about 5% chemical precursors or non-PTMAX chemicals.

Biologically active portions of a PTMAX protein include peptides comprising amino acid sequences sufficiently homologous to or derived from the amino acid sequence of the PTMAX protein, e.g., the amino acid sequence shown in SEQ ID NO:2, 4, 6, 8, 10, 12, 14, 16, 18, 20, 22, 24, 26 or 52, that include fewer amino acids than the fall length PTMAX proteins, and exhibit at least one activity of an PTMAX protein. Typically, biologically active portions comprise a domain or motif with at least one activity of the PTMAX protein. A biologically active portion of a PTMAX protein can be a polypeptide which is, for example, 10, 25, 50, 100 or more amino acids in length.

Moreover, other biologically active portions, in which other regions of the protein are deleted, can be prepared by recombinant techniques and evaluated for one or more of the functional activities of a native PTMAX protein.

In an embodiment, the PTMAX protein has an amino acid sequence shown in SEQ ID NO:2, 4, 6, 8, 10, 12, 14, 16, 18, 20, 22, 24, 26 or 52. In other embodiments, the PTMAX protein is substantially homologous to SEQ ID NO:2, 4, 6, 8, 10, 12, 14, 16, 18, 20, 22, 24, 26 or 52 and retains the functional activity of the protein of SEQ ID NO:2, 4, 6, 8, 10, 12, 14, 16, 18, 20, 22, 24, 26 or 52 yet differs in amino acid sequence due to natural allelic variation or mutagenesis, as described in detail below. Accordingly, in another embodiment, the PTMAX protein is a protein that comprises an amino acid sequence at least about 45% homologous to the amino acid sequence of SEQ ID NO:2, 4, 6, 8, 10, 12, 14, 16, 18, 20, 22, 24, 26 or 52 and retains the functional activity of the PTMAX proteins of SEQ ID NO:2, 4, 6, 8, 10, 12, 14, 16, 18, 20, 22, 24, 26 or 52.

### Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch 1970 J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NO:1, 3, 5, 7, 9, 11, 13, 15, 17, 19, 21, 23, 25 or 51.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

### Chimeric and Fusion Proteins

The invention also provides PTMAX chimeric or fusion proteins. As used herein, an PTMAX “chimeric protein” or “fusion protein” comprises a PTMAX polypeptide operatively linked to a non-PTMAX polypeptide. A “PTMAX polypeptide” refers to a polypeptide having an amino acid sequence corresponding to PTMAX, whereas a “non-PTMAX polypeptide” refers to a polypeptide having an amino acid sequence corresponding to a protein that is not substantially homologous to the PTMAX protein, e.g., a protein that is different from the PTMAX protein and that is derived from the same or a different organism. Within a PTMAX fusion protein the PTMAX polypeptide can correspond to all or a portion of a PTMAX protein. In one embodiment, a PTMAX fusion protein comprises at least one biologically active portion of a PTMAX protein. In another embodiment, a PTMAX fusion protein comprises at least two biologically active portions of a PTMAX protein. In yet another embodiment, a PTMAX fusion protein comprises at least three biologically active portions of a PTMAX protein. Within the fusion protein, the term operatively linked“is intended to indicate that the PTMAX polypeptide and the non-PTMAX polypeptide are fused in-frame to each other. The non-PTMAX polypeptide can be fused to the N-terminus or C-terminus of the PTMAX polypeptide.

In one embodiment, the fusion protein is a GST-PTMAX fusion protein in which the PTMAX sequences are fused to the C-terminus of the GST (i.e., glutathione S-transferase) sequences. Such fusion proteins can facilitate the purification of recombinant PTMAX.

In another embodiment, the fusion protein is a PTMAX protein containing a heterologous signal sequence at its N-terminus. For example, the native PTMA-7 signal sequence (i.e., about amino acids 1 to 40 of SEQ ID NO:14) can be removed and replaced with a signal sequence from another protein. In certain host cells (e.g., mammalian host cells), expression and/or secretion of PTMAX can be increased through use of a heterologous signal sequence.

In yet another embodiment, the fusion protein is a PTMAX-immunoglobulin fusion protein in which the PTMAX sequences are fused to sequences derived from a member of the immunoglobulin protein family. The PTMAX-immunoglobulin fusion proteins of the invention can be incorporated into pharmaceutical compositions and administered to a subject to inhibit an interaction between a PTMAX ligand and an PTMAX protein on the surface of a cell, to thereby suppress PTMAX-mediated signal transduction in vivo. The PTMAX-immunoglobulin fusion proteins can be used to affect the bioavailability of a PTMAX cognate ligand. Inhibition of the PTMAX ligand/PTMAX interaction may be useful therapeutically for both the treatment of proliferative and differentiative disorders, as well as modulating (e.g. promoting or inhibiting) cell survival. Moreover, the PTMAX-immunoglobulin fusion proteins of the invention can be used as immunogens to produce anti-PTMAX antibodies in a subject, to purify PTMAX ligands, and in screening assays to identify molecules that inhibit the interaction of PTMAX with an PTMAX ligand.

A PTMAX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, for example, Ausubel et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). A PTMAX-encoding nucleic acid can be cloned into such an expression vector such that the fusion moiety is linked in-frame to the PTMAX protein.

### PTMAX Agonists and Antagonists

The present invention also pertains to variants of the PTMAX proteins that function as either PTMAX agonists (mimetics) or as PTMAX antagonists. Variants of the PTMAX protein can be generated by mutagenesis, e.g., discrete point mutation or truncation of the PTMAX protein. An agonist of the PTMAX protein can retain substantially the same, or a subset of, the biological activities of the naturally occurring form of the PTMAX protein. An antagonist of the PTMAX protein can inhibit one or more of the activities of the naturally occurring form of the PTMAX protein by, for example, competitively binding to a downstream or upstream member of a cellular signaling cascade which includes the PTMAX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the PTMAX proteins.

Variants of the PTMAX protein that function as either PTMAX agonists (mimetics) or as PTMAX antagonists can be identified by screening combinatorial libraries of mutants, e.g., truncation mutants, of the PTMAX protein for PTMAX protein agonist or antagonist activity. In one embodiment, a variegated library of PTMAX variants is generated by combinatorial mutagenesis at the nucleic acid level and is encoded by a variegated gene library. A variegated library of PTMAX variants can be produced by, for example, enzymatically ligating a mixture of synthetic oligonucleotides into gene sequences such that a degenerate set of potential PTMAX sequences is expressible as individual polypeptides, or alternatively, as a set of larger fusion proteins (e.g., for phage display) containing the set of PTMAX sequences therein. There are a variety of methods which can be used to produce libraries of potential PTMAX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential PTMAX sequences. Methods for synthesizing degenerate oligonucleotides are known in the art (see, e.g., Narang (1983) Tetrahedron 39:3; Itakura et al. (1984) Annu Rev Biochem 53:323; Itakura et al. (1984) Science 198:1056; Ike et al. (1983) Nucl Acid Res 11:477.

### Polypeptide Libraries

In addition, libraries of fragments of the PTMAX protein coding sequence can be used to generate a variegated population of PTMAX fragments for screening and subsequent selection of variants of a PTMAX protein. In one embodiment, a library of coding sequence fragments can be generated by treating a double stranded PCR fragment of an PTMAX coding sequence with a nuclease under conditions wherein nicking occurs only about once per molecule, denaturing the double stranded DNA, renaturing the DNA to form double stranded DNA that can include sense/antisense pairs from different nicked products, removing single stranded portions from reformed duplexes by treatment with S1 nuclease, and ligating the resulting fragment library into an expression vector. By this method, an expression library can be derived which encodes N-terminal and internal fragments of various sizes of the PTMAX protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of PTMAX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify PTMAX variants (Arkin and Yourvan (1992) PNAS 89:7811-7815; Delgrave et al. (1993) Protein Engineering 6:327-331).

### Anti-PTMAX Antibodies

Also included in the invention are antibodies to PTMAX proteins, or fragments of PTMAX proteins. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′} and F_{(ab′)2} fragments, and an F_{ab} expression library. In general, an antibody molecule obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated PTMAX-related protein of the invention may be intended to serve as an antigen, or a portion or fragment thereof, and additionally can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of PTMAX-related protein that is located on the surface of the protein, e.g., a hydrophilic region. A hydrophobicity analysis of the human PTMAX-related protein sequence will indicate which regions of a PTMAX-related protein are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each of which is incorporated herein by reference in its entirety. Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow and Lane, 1988, Cold Spring Harbor Laboratory Press, Cold Spring Harbor, N.Y., incorporated herein by reference). Some of these antibodies are discussed below.

### Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

### Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, MONOCLONAL ANTIBODIES: PRINCIPLES AND PRACTICE, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., MONOCLONAL ANTIBODY PRODUCTION TECHNIQUES AND APPLICATIONS, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Preferably, antibodies having a high degree of specificity and a high binding affinity for the target antigen are isolated.

After the desired hybridoma cells are identified, the clones can be subcloned by limiting dilution procedures and grown by standard methods. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

### Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

### Human Antibodies

Fully human antibodies relate to antibody molecules in which essentially the entire sequences of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al,(Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

### F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of Fab expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

### Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., 1991 EMBO J., 10:3655-3659.

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

### Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

### Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

### Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of an PTMAX protein is facilitated by generation of hybridomas that bind to the fragment of an PTMAX protein possessing such a domain. Thus, antibodies that are specific for a desired domain within an PTMAX protein, or derivatives, fragments, analogs or homologs thereof, are also provided herein.

Anti-PTMAX antibodies may be used in methods known within the art relating to the localization and/or quantitation of an PTMAX protein (e.g., for use in measuring levels of the PTMAX protein within appropriate physiological samples, for use in diagnostic methods, for use in imaging the protein, and the like). In a given embodiment, antibodies for PTMAX proteins, or derivatives, fragments, analogs or homologs thereof, that contain the antibody derived binding domain, are utilized as pharmacologically-active compounds (hereinafter “Therapeutics”).

An anti-PTMAX antibody (e.g., monoclonal antibody) can be used to isolate an PTMAX polypeptide by standard techniques, such as affinity chromatography or immunoprecipitation. An anti-PTMAX antibody can facilitate the purification of natural PTMAX polypeptide from cells and of recombinantly-produced PTMAX polypeptide expressed in host cells. Moreover, an anti-PTMAX antibody can be used to detect PTMAX protein (e.g., in a cellular lysate or cell supernatant) in order to evaluate the abundance and pattern of expression of the PTMAX protein. Anti-PTMAX antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

### PTMAX Recombinant Expression Vectors and Host Cells

Another aspect of the invention pertains to vectors, preferably expression vectors, containing a nucleic acid encoding PTMAX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively linked to the nucleic acid sequence to be expressed. Within a recombinant expression vector, “operably linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cells and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., PTMAX proteins, mutant forms of PTMAX, fusion proteins, etc.).

The recombinant expression vectors of the invention can be designed for expression of PTMAX in prokaryotic or eukaryotic cells. For example, PTMAX can be expressed in bacterial cells such as E. coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (1) to increase expression of recombinant protein; (2) to increase the solubility of the recombinant protein; and (3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene 67:31-40), pMAL (New England Biolabs, Beverly, Mass.) and pRIT5 (Phamacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the PTMAX expression vector is a yeast expression vector. Examples of vectors for expression in yeast S. cerivisae include pYepSec1 (Baldari, et al., (1987) EMBO J 6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.).

Alternatively, PTMAX can be expressed in insect cells using baculovirus expression vectors. Baculovirus vectors available for expression of proteins in cultured insect cells (e.g., SF9 cells) include the pAc series (Smith et al. (1983) Mol Cell Biol 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed (1987) Nature 329:840) and pMT2PC (Kaufman et al. (1987) EMBO J 6: 187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see, e.g., Chapters 16 and 17 of Sambrook et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev 1:268-277), lymphoid-specific promoters (Calame and Eaton (1988) Adv Immunol 43:235-275), in particular promoters of T cell receptors (Winoto and Baltimore (1989) EMBO J 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) PNAS 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss (1990) Science 249:374-379) and the α-fetoprotein promoter (Campes and Tilghman (1989) Genes Dev 3:537-546).

The invention further provides a recombinant expression vector comprising a DNA molecule of the invention cloned into the expression vector in an antisense orientation. That is, the DNA molecule is operatively linked to a regulatory sequence in a manner that allows for expression (by transcription of the DNA molecule) of an RNA molecule that is antisense to PTMAX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al., “Antisense RNA as a molecular tool for genetic analysis,” Reviews—Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, PTMAX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding PTMAX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) PTMAX protein. Accordingly, the invention further provides methods for producing PTMAX protein using the host cells of the invention. In one embodiment, the method comprises culturing the host cell of invention (into which a recombinant expression vector encoding PTMAX has been introduced) in a suitable medium such that PTMAX protein is produced. In another embodiment, the method further comprises isolating PTMAX from the medium or the host cell.

### Transgenic Animals

The host cells of the invention can also be used to produce nonhuman transgenic animals. For example, in one embodiment, a host cell of the invention is a fertilized oocyte or an embryonic stem cell into which PTMAX-coding sequences have been introduced. Such host cells can then be used to create non-human transgenic animals in which exogenous PTMAX sequences have been introduced into their genome or homologous recombinant animals in which endogenous PTMAX sequences have been altered. Such animals are useful for studying the function and/or activity of PTMAX and for identifying and/or evaluating modulators of PTMAX activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous PTMAX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing PTMAX-encoding nucleic acid into the male pronuclei of a fertilized oocyte, e.g., by microinjection, retroviral infection, and allowing the oocyte to develop in a pseudopregnant female foster animal. The human PTMAX cDNA sequence of SEQ ID NO:1, 3, 5, 7, 9, 11, 13, 15, 17, 19, 21, 23, 25 or 51 can be introduced as a transgene into the genome of a non-human animal. Alternatively, a nonhuman homologue of the human PTMAX gene, such as a mouse PTMAX gene, can be isolated based on hybridization to the human PTMAX cDNA (described further above) and used as a transgene. Intronic sequences and polyadenylation signals can also be included in the transgene to increase the efficiency of expression of the transgene. A tissue-specific regulatory sequence(s) can be operably linked to the PTMAX transgene to direct expression of PTMAX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan 1986, In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the PTMAX transgene in its genome and/or expression of PTMAX mRNA in tissues or cells of the animals. A transgenic founder animal can then be used to breed additional animals carrying the transgene. Moreover, transgenic animals carrying a transgene encoding PTMAX can further be bred to other transgenic animals carrying other transgenes.

To create a homologous recombinant animal, a vector is prepared which contains at least a portion of a PTMAX gene into which a deletion, addition or substitution has been introduced to thereby alter, e.g., functionally disrupt, the PTMAX gene. The PTMAX gene can be a human gene (e.g., the cDNA of SEQ ID NO:1, 3, 5, 7, 9, 11, 13, 15, 17, 19, 21, 23, 25 or 51), but more preferably, is a non-human homologue of a human PTMAX gene. For example, a mouse homologue of human PTMAX gene of SEQ ID NO:1, 3, 5, 7, 9, 11, 13, 15, 17, 19, 21, 23, 25 or 51 can be used to construct a homologous recombination vector suitable for altering an endogenous PTMAX gene in the mouse genome. In one embodiment, the vector is designed such that, upon homologous recombination, the endogenous PTMAX gene is functionally disrupted (i.e., no longer encodes a functional protein; also referred to as a “knock out” vector).

Alternatively, the vector can be designed such that, upon homologous recombination, the endogenous PTMAX gene is mutated or otherwise altered but still encodes functional protein (e.g., the upstream regulatory region can be altered to thereby alter the expression of the endogenous PTMAX protein). In the homologous recombination vector, the altered portion of the PTMAX gene is flanked at its 5′ and 3′ ends by additional nucleic acid of the PTMAX gene to allow for homologous recombination to occur between the exogenous PTMAX gene carried by the vector and an endogenous PTMAX gene in an embryonic stem cell. The additional flanking PTMAX nucleic acid is of sufficient length for successful homologous recombination with the endogenous gene. Typically, several kilobases of flanking DNA (both at the 5′ and 3′ ends) are included in the vector. See e.g., Thomas et al. (1987) Cell 51:503 for a description of homologous recombination vectors. The vector is introduced into an embryonic stem cell line (e.g., by electroporation) and cells in which the introduced PTMAX gene has homologously recombined with the endogenous PTMAX gene are selected (see e.g., Li et al. (1992) Cell 69:915).

The selected cells are then injected into a blastocyst of an animal (e.g., a mouse) to form aggregation chimeras. See e.g., Bradley 1987, In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley (1991) Curr Opin Biotechnol 2:823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, see, e.g., Lakso et al. (1992) PNAS 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut et al. (1997) Nature 385:810-813. In brief, a cell, e.g., a somatic cell, from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

### Pharmaceutical Compositions

The PTMAX nucleic acid molecules, PTMAX proteins, and anti-PTMAX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an PTMAX protein or anti-PTMAX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) PNAS 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

### Uses and Methods of the Invention

The isolated nucleic acid molecules of the invention can be used to express PTMAX protein (e.g., via a recombinant expression vector in a host cell in gene therapy applications), to detect PTMAX mRNA (e.g., in a biological sample) or a genetic lesion in an PTMAX gene, and to modulate PTMAX activity, as described further below. In addition, the PTMAX proteins can be used to screen drugs or compounds that modulate the PTMAX activity or expression as well as to treat disorders characterized by insufficient or excessive production of PTMAX protein or production of PTMAX protein forms that have decreased or aberrant activity compared to PTMAX wild type protein (e.g. proliferative disorders such as cancer and immune disorders, e.g., multiple sclerosis). In addition, the anti-PTMAX antibodies of the invention can be used to detect and isolate PTMAX proteins and modulate PTMAX activity.

This invention further pertains to novel agents identified by the screening assays described herein and uses thereof for treatments as described herein.

### Screening Assays

The invention provides a method (also referred to herein as a “screening assay”) for identifying modulators, i.e., candidate or test compounds or agents (e.g., peptides, peptidomimetics, small molecules or other drugs) that bind to PTMAX proteins or have a stimulatory or inhibitory effect on, for example, PTMAX expression or PTMAX activity. The invention also includes compounds identified in the screening assays described herein.

In one embodiment, the invention provides assays for screening candidate or test compounds which bind to or modulate the activity of the membrane-bound form of a PTMAX protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des 12:145).

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic (carbon containing) or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt et al. (1993) Proc Natl Acad Sci U.S.A. 90:6909; Erb et al. (1994) Proc Natl Acad Sci U.S.A. 91:11422; Zuckermann et al. (1994) J Med Chem 37:2678; Cho et al. (1993) Science 261:1303; Carrell et al. (1994) Angew Chem Int Ed Engl 33:2059; Carell et al. (1994) Angew Chem Int Ed Engl 33:2061; and Gallop et al. (1994) J Med Chem 37:1233.

Libraries of compounds may be presented in solution (e.g., Houghten (1992) Biotechniques 13:412-421), or on beads (Lam (1991) Nature 354:82-84), on chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc Natl Acad Sci U.S.A. 87:6378-6382; Felici (1991) J Mol Bio 222:301-310; Ladner above.).

In one embodiment, an assay is a cell-based assay in which a cell which expresses a membrane-bound form of PTMAX protein, or a biologically active portion thereof, on the cell surface is contacted with a test compound and the ability of the test compound to bind to a PTMAX protein is determined. The cell, for example, can be of mammalian origin or a yeast cell. Determining the ability of the test compound to bind to the PTMAX protein can be accomplished, for example, by coupling the test compound with a radioisotope or enzymatic label such that binding of the test compound to the PTMAX protein or biologically active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically labeled with, for example, horseradish peroxidase, alkaline phosphatase, or luciferase, and the enzymatic label detected by determination of conversion of an appropriate substrate to product.

In one embodiment, the assay comprises contacting a cell which expresses a membrane-bound form of PTMAX protein, or a biologically active portion thereof, on the cell surface with a known compound which binds PTMAX to form an assay mixture, contacting the assay mixture with a test compound, and determining the ability of the test compound to interact with a PTMAX protein, wherein determining the ability of the test compound to interact with a PTMAX protein comprises determining the ability of the test compound to preferentially bind to PTMAX or a biologically active portion thereof as compared to the known compound.

In another embodiment, an assay is a cell-based assay comprising contacting a cell expressing a membrane-bound form of PTMAX protein, or a biologically active portion thereof, on the cell surface with a test compound and determining the ability of the test compound to modulate (e.g., stimulate or inhibit) the activity of the PTMAX protein or biologically active portion thereof. Determining the ability of the test compound to modulate the activity of PTMAX or a biologically active portion thereof can be accomplished, for example, by determining the ability of the PTMAX protein to bind to or interact with an PTMAX target molecule. As used herein, a “target molecule” is a molecule with which an PTMAX protein binds or interacts in nature, for example, a molecule on the surface of a cell which expresses an PTMAX interacting protein, a molecule on the surface of a second cell, a molecule in the extracellular milieu, a molecule associated with the internal surface of a cell membrane, a molecule associated with the nuclear membrane, a molecule in the nucleus, or a cytoplasmic molecule. A PTMAX target molecule can be a non-PTMAX molecule or a PTMAX protein or polypeptide of the present invention.

In one embodiment, a PTMAX target molecule is a component of a signal transduction pathway that facilitates transduction of an extracellular signal (e.g. a signal generated by binding of a compound to a membrane-bound PTMAX molecule) through the cell membrane and into the cell. The target, for example, can be a second intercellular protein that has catalytic activity or a protein that facilitates the association of downstream signaling molecules with PTMAX.

Determining the ability of the PTMAX protein to bind to or interact with a PTMAX target molecule can be accomplished by one of the methods described above for determining direct binding. In one embodiment, determining the ability of the PTMAX protein to bind to or interact with a PTMAX target molecule can be accomplished by determining the activity of the target molecule. For example, the activity of the target molecule can be determined by detecting induction of a cellular second messenger of the target (i.e. intracellular Ca²+, diacylglycerol, IP₃, etc.), detecting catalytic/enzymatic activity of the target an appropriate substrate, detecting the induction of a reporter gene (comprising a PTMAX-responsive regulatory element operatively linked to a nucleic acid encoding a detectable marker, e.g., luciferase), or detecting a cellular response, for example, cell survival, cell death, cellular differentiation, or cell proliferation.

In yet another embodiment, an assay of the present invention is a cell-free assay comprising contacting a PTMAX protein or biologically active portion thereof with a test compound and determining the ability of the test compound to bind to the PTMAX protein or biologically active portion thereof. Binding of the test compound to the PTMAX protein can be determined either directly or indirectly as described above. In one embodiment, the assay comprises contacting the PTMAX protein or biologically active portion thereof with a known compound which binds PTMAX to form an assay mixture, contacting the assay mixture with a test compound, and determining the ability of the test compound to interact with a PTMAX protein, wherein determining the ability of the test compound to interact with a PTMAX protein comprises determining the ability of the test compound to preferentially bind to PTMAX or biologically active portion thereof as compared to the known compound.

In another embodiment, an assay is a cell-free assay comprising contacting PTMAX protein or biologically active portion thereof with a test compound and determining the ability of the test compound to modulate (e.g. stimulate or inhibit) the activity of the PTMAX protein or biologically active portion thereof. Determining the ability of the test compound to modulate the activity of PTMAX can be accomplished, for example, by determining the ability of the PTMAX protein to bind to a PTMAX target molecule by one of the methods described above for determining direct binding. In an alternative embodiment, determining the ability of the test compound to modulate the activity of PTMAX can be accomplished by determining the ability of the PTMAX protein to further modulate a PTMAX target molecule. For example, the catalytic/enzymatic activity of the target molecule on an appropriate substrate can be determined as previously described.

In yet another embodiment, the cell-free assay comprises contacting the PTMAX protein or biologically active portion thereof with a known compound which binds PTMAX to form an assay mixture, contacting the assay mixture with a test compound, and determining the ability of the test compound to interact with a PTMAX protein, wherein determining the ability of the test compound to interact with a PTMAX protein comprises determining the ability of the PTMAX protein to preferentially bind to or modulate the activity of a PTMAX target molecule.

The cell-free assays of the present invention are amenable to use of both the soluble form or the membrane-bound form of PTMAX. In the case of cell-free assays comprising the membrane-bound form of PTMAX, it may be desirable to utilize a solubilizing agent such that the membrane-bound form of PTMAX is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl-N,N-dimethyl-3-ammonio-1-propane sulfonate, 3-(3-cholamidopropyl)dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the present invention, it may be desirable to immobilize either PTMAX or its target molecule to facilitate separation of complexed from uncomplexed forms of one or both of the proteins, as well as to accommodate automation of the assay. Binding of a test compound to PTMAX, or interaction of PTMAX with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-PTMAX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or PTMAX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of PTMAX binding or activity determined using standard techniques.

Other techniques for immobilizing proteins on matrices can also be used in the screening assays of the invention. For example, either PTMAX or its target molecule can be immobilized utilizing conjugation of biotin and streptavidin. Biotinylated PTMAX or target molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with PTMAX or target molecules, but which do not interfere with binding of the PTMAX protein to its target molecule, can be derivatized to the wells of the plate, and unbound target or PTMAX trapped in the wells by antibody conjugation. Methods for detecting such complexes, in addition to those described above for the GST-immobilized complexes, include immunodetection of complexes using antibodies reactive with the PTMAX or target molecule, as well as enzyme-linked assays that rely on detecting an enzymatic activity associated with the PTMAX or target molecule.

In another embodiment, modulators of PTMAX expression are identified in a method wherein a cell is contacted with a candidate compound and the expression of PTMAX mRNA or protein in the cell is determined. The level of expression of PTMAX mRNA or protein in the presence of the candidate compound is compared to the level of expression of PTMAX mRNA or protein in the absence of the candidate compound. The candidate compound can then be identified as a modulator of PTMAX expression based on this comparison. For example, when expression of PTMAX mRNA or protein is greater (statistically significantly greater) in the presence of the candidate compound than in its absence, the candidate compound is identified as a stimulator of PTMAX mRNA or protein expression. Alternatively, when expression of PTMAX mRNA or protein is less (statistically significantly less) in the presence of the candidate compound than in its absence, the candidate compound is identified as an inhibitor of PTMAX mRNA or protein expression. The level of PTMAX mRNA or protein expression in the cells can be determined by methods described herein for detecting PTMAX mRNA or protein.

In yet another aspect of the invention, the PTMAX proteins can be used as “bait proteins” in a two-hybrid assay or three hybrid assay (see, e.g., U.S. Pat. No. 5,283,317; Zervos et al. (1993) Cell 72:223-232; Madura et al. (1993) J Biol Chem 268:12046-12054; Bartel et al. (1993) Biotechniques 14:920-924; Iwabuchi et al. (1993) Oncogene 8:1693-1696; and Brent WO94/10300), to identify other proteins that bind to or interact with PTMAX (“PTMAX-binding proteins” or “PTMAX-bp”) and modulate PTMAX activity. Such PTMAX-binding proteins are also likely to be involved in the propagation of signals by the PTMAX proteins as, for example, upstream or downstream elements of the PTMAX pathway.

The two-hybrid system is based on the modular nature of most transcription factors, which consist of separable DNA-binding and activation domains. Briefly, the assay utilizes two different DNA constructs. In one construct, the gene that codes for PTMAX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a PTMAX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with PTMAX.

This invention further pertains to novel agents identified by the above-described screening assays and uses thereof for treatments as described herein.

### Chromosome Mapping

Once the sequence (or a portion of the sequence) of a gene has been isolated, this sequence can be used to map the location of the gene on a chromosome. This process is called chromosome mapping. Accordingly, portions or fragments of the PTMAX, sequences, described herein, can be used to map the location of the PTMAX genes, respectively, on a chromosome. The mapping of the PTMAX sequences to chromosomes is an important first step in correlating these sequences with genes associated with disease.

Briefly, PTMAX genes can be mapped to chromosomes by preparing PCR primers (preferably 15-25 bp in length) from the PTMAX sequences. Computer analysis of the PTMAX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the PTMAX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. (D'Eustachio et al. (1983) Science 220:919-924). Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the PTMAX sequences to design oligonucleotide primers, sublocalization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see Venna et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, for example, in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, for example, Egeland et al. (1987) Nature, 325:783-787.

Moreover, differences in the DNA sequences between individuals affected and unaffected with a disease associated with the PTMAX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

### Tissue Typing

The PTMAX sequences of the present invention can also be used to identify individuals from minute biological samples. In this technique, an individual's genomic DNA is digested with one or more restriction enzymes, and probed on a Southern blot to yield unique bands for identification. The sequences of the present invention are useful as additional DNA markers for RFLP (“restriction fragment length polymorphisms,” described in U.S. Pat. No. 5,272,057).

Furthermore, the sequences of the present invention can be used to provide an alternative technique that determines the actual base-by-base DNA sequence of selected portions of an individual's genome. Thus, the PTMAX sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The PTMAX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO:1, 3, 5 or 7 can comfortably provide positive individual identification with a panel of perhaps 10 to 1,000 primers that each yield a noncoding amplified sequence of 100 bases. If predicted coding sequences, such as those in SEQ ID NO:9, 10, 11, or 12 are used, a more appropriate number of primers for positive individual identification would be 500-2,000.

### Predictive Medicine

The present invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the present invention relates to diagnostic assays for determining PTMAX protein and/or nucleic acid expression as well as PTMAX activity, in the context of a biological sample (e.g., blood, serum, cells, tissue) to thereby determine whether an individual is afflicted with a disease or disorder, or is at risk of developing a disorder, associated with aberrant PTMAX expression or activity. The invention also provides for prognostic (or predictive) assays for determining whether an individual is at risk of developing a disorder associated with PTMAX protein, nucleic acid expression or activity. For example, mutations in a PTMAX gene can be assayed in a biological sample. Such assays can be used for prognostic or predictive purpose to thereby prophylactically treat an individual prior to the onset of a disorder characterized by or associated with PTMAX protein, nucleic acid expression or activity.

Another aspect of the invention provides methods for determining PTMAX protein, nucleic acid expression or PTMAX activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of PTMAX in clinical trials.

These and other agents are described in further detail in the following sections.

### Diagnostic Assays

An exemplary method for detecting the presence or absence of PTMAX in a biological sample involves obtaining a biological sample from a test subject and contacting the biological sample with a compound or an agent capable of detecting PTMAX protein or nucleic acid (e.g., mRNA, genomic DNA) that encodes PTMAX protein such that the presence of PTMAX is detected in the biological sample. An agent for detecting PTMAX mRNA or genomic DNA is a labeled nucleic acid probe capable of hybridizing to PTMAX mRNA or genomic DNA. The nucleic acid probe can be, for example, a full-length PTMAX nucleic acid, such as the nucleic acid of SEQ ID NO:1, 3, 5, 7, 9, 11, 13, 15, 17, 19, 21, 23, 25 OR 51 or a portion thereof, such as an oligonucleotide of at least 15, 30, 50, 100, 250 or 500 nucleotides in length and sufficient to specifically hybridize under stringent conditions to PTMAX mRNA or genomic DNA. Other suitable probes for use in the diagnostic assays of the invention are described herein.

An agent for detecting PTMAX protein is an antibody capable of binding to PTMAX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect PTMAX mRNA, protein, or genomic DNA in a biological sample in vitro as well as in vivo. For example, in vitro techniques for detection of PTMAX mRNA include Northern hybridizations and in situ hybridizations. In vitro techniques for detection of PTMAX protein include enzyme linked immunosorbent assays (ELISAs), Western blots, immunoprecipitations and immunofluorescence. In vitro techniques for detection of PTMAX genomic DNA include Southern hybridizations. Furthermore, in vivo techniques for detection of PTMAX protein include introducing into a subject a labeled anti-PTMAX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting PTMAX protein, mRNA, or genomic DNA, such that the presence of PTMAX protein, mRNA or genomic DNA is detected in the biological sample, and comparing the presence of PTMAX protein, mRNA or genomic DNA in the control sample with the presence of PTMAX protein, mRNA or genomic DNA in the test sample.

The invention also encompasses kits for detecting the presence of PTMAX in a biological sample. For example, the kit can comprise: a labeled compound or agent capable of detecting PTMAX protein or mRNA in a biological sample; means for determining the amount of PTMAX in the sample; and means for comparing the amount of PTMAX in the sample with a standard. The compound or agent can be packaged in a suitable container. The kit can further comprise instructions for using the kit to detect PTMAX protein or nucleic acid.

### Prognostic Assays

The diagnostic methods described herein can furthermore be utilized to identify subjects having or at risk of developing a disease or disorder associated with aberrant PTMAX expression or activity. For example, the assays described herein, such as the preceding diagnostic assays or the following assays, can be utilized to identify a subject having or at risk of developing a disorder associated with PTMAX protein, nucleic acid expression or activity such as cancer, immune system associated (e.g., multiple sclerosis), or fibrotic disorders. Alternatively, the prognostic assays can be utilized to identify a subject having or at risk for developing a disease or disorder. Thus, the present invention provides a method for identifying a disease or disorder associated with aberrant PTMAX expression or activity in which a test sample is obtained from a subject and PTMAX protein or nucleic acid (e.g., mRNA, genomic DNA) is detected, wherein the presence of PTMAX protein or nucleic acid is diagnostic for a subject having or at risk of developing a disease or disorder associated with aberrant PTMAX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant PTMAX expression or activity. For example, such methods can be used to determine whether a subject can be effectively treated with an agent for a disorder, such as cancer, immune system associated disorders, e.g., multiple sclerosis. Thus, the present invention provides methods for determining whether a subject can be effectively treated with an agent for a disorder associated with aberrant PTMAX expression or activity in which a test sample is obtained and PTMAX protein or nucleic acid is detected (e.g., wherein the presence of PTMAX protein or nucleic acid is diagnostic for a subject that can be administered the agent to treat a disorder associated with aberrant PTMAX expression or activity.)

The methods of the invention can also be used to detect genetic lesions in an PTMAX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding an PTMAX-protein, or the mis-expression of the PTMAX gene. For example, such genetic lesions can be detected by ascertaining the existence of at least one of (1) a deletion of one or more nucleotides from an PTMAX gene; (2) an addition of one or more nucleotides to an PTMAX gene; (3) a substitution of one or more nucleotides of an PTMAX gene, (4) a chromosomal rearrangement of an PTMAX gene; (5) an alteration in the level of a messenger RNA transcript of an PTMAX gene, (6) aberrant modification of an PTMAX gene, such as of the methylation pattern of the genomic DNA, (7) the presence of a non-wild type splicing pattern of a messenger RNA transcript of an PTMAX gene, (8) a non-wild type level of an PTMAX-protein, (9) allelic loss of an PTMAX gene, and (10) inappropriate post-translational modification of an PTMAX-protein. As described herein, there are a large number of assay techniques known in the art which can be used for detecting lesions in an PTMAX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) PNAS 91:360-364), the latter of which can be particularly useful for detecting point mutations in the PTMAX-gene (see Abravaya et al. (1995) Nucl Acids Res 23:675-682). This method can include the steps of collecting a sample of cells from a patient, isolating nucleic acid (e.g., genomic, mRNA or both) from the cells of the sample, contacting the nucleic acid sample with one or more primers that specifically hybridize to an PTMAX gene under conditions such that hybridization and amplification of the PTMAX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli et al., 1990, Proc Natl Acad Sci USA 87:1874-1878), transcriptional amplification system (Kwoh, et al., 1989, Proc Natl Acad Sci USA 86:1173-1177), Q-Beta Replicase (Lizardi et al., 1988, BioTechnology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a PTMAX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,493,531) can be used to score for the presence of specific mutations by development or loss of a ribozyme cleavage site.

In other embodiments, genetic mutations in PTMAX can be identified by hybridizing a sample and control nucleic acids, e.g., DNA or RNA, to high density arrays containing hundreds or thousands of oligonucleotides probes (Cronin et al. (1996) Human Mutation 7: 244-255; Kozal et al. (1996) Nature Medicine 2: 753-759). For example, genetic mutations in PTMAX can be identified in two dimensional arrays containing light-generated DNA probes as described in Cronin et al. above. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the PTMAX gene and detect mutations by comparing the sequence of the sample PTMAX with the corresponding wild-type (control) sequence. Examples of sequencing reactions include those based on techniques developed by Maxim and Gilbert (1977) PNAS 74:560 or Sanger (1977) PNAS 74:5463. It is also contemplated that any of a variety of automated sequencing procedures can be utilized when performing the diagnostic assays (Naeve et al., (1995) Biotechniques 19:448), including sequencing by mass spectrometry (see, e.g., PCT International Publ. No. WO 94/16101; Cohen et al. (1996) Adv Chromatogr 36:127-162; and Griffin et al. (1993) Appl Biochem Biotechnol 38:147-159).

Other methods for detecting mutations in the PTMAX gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes of formed by hybridizing (labeled) RNA or DNA containing the wild-type PTMAX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al (1988) Proc Natl Acad Sci USA 85:4397; Saleeba et al (1992) Methods Enzymol 217:286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in PTMAX cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a PTMAX sequence, e.g., a wild-type PTMAX sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in PTMAX genes. For example, single strand conformation polymorphism (SSCP) may be used to detect differences in electrophoretic mobility between mutant and wild type nucleic acids (Orita et al. (1989) Proc Natl Acad Sci USA: 86:2766, see also Cotton (1993) Mutat Res 285:125-144; Hayashi (1992) Genet Anal Tech Appl 9:73-79). Single-stranded DNA fragments of sample and control PTMAX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility (Keen et al. (1991) Trends Genet 7:5).

In yet another embodiment the movement of mutant or wild-type fragments in polyacrylamide gels containing a gradient of denaturant is assayed using denaturing gradient gel electrophoresis (DGGE) (Myers et al (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found (Saiki et al. (1986) Nature 324:163); Saiki et al. (1989) Proc Natl Acad. Sci USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al (1992) Mol Cell Probes 6:1). It is anticipated that in certain embodiments amplification may also be performed using Taq ligase for amplification (Barany (1991) Proc Natl Acad Sci USA 88:189). In such cases, ligation will occur only if there is a perfect match at the 3′ end of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an PTMAX gene.

Furthermore, any cell type or tissue, preferably thymus tissue, in which PTMAX is expressed may be utilized in the prognostic assays described herein. However, any biological sample containing nucleated cells may be used, including, for example, buccal mucosal cells.

### Pharmacogenomics

Agents, or modulators that have a stimulatory or inhibitory effect on PTMAX activity (e.g., PTMAX gene expression), as identified by a screening assay described herein can be administered to individuals to treat (prophylactically or therapeutically) disorders (e.g., cancer or immune disorders associated with aberrant PTMAX activity. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of PTMAX protein, expression of PTMAX nucleic acid, or mutation content of PTMAX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, Clin Exp Pharmacol Physiol, 1996, 23:983-985 and Linder, Clin Chem, 1997, 43:254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is haemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C 19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of PTMAX protein, expression of PTMAX nucleic acid, or mutation content of PTMAX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with an PTMAX modulator, such as a modulator identified by one of the exemplary screening assays described herein.

### Monitoring of Effects During Clinical Trials

Monitoring the influence of agents (e.g., drugs, compounds) on the expression or activity of PTMAX (e.g., the ability to modulate aberrant cell proliferation and/or differentiation) can be applied not only in basic drug screening, but also in clinical trials. For example, the effectiveness of an agent determined by a screening assay as described herein to increase PTMAX gene expression, protein levels, or upregulate PTMAX activity, can be monitored in clinical trails of subjects exhibiting decreased PTMAX gene expression, protein levels, or downregulated PTMAX activity. Alternatively, the effectiveness of an agent determined by a screening assay to decrease PTMAX gene expression, protein levels, or downregulate PTMAX activity, can be monitored in clinical trails of subjects exhibiting increased PTMAX gene expression, protein levels, or upregulated PTMAX activity. In such clinical trials, the expression or activity of PTMAX and, preferably, other genes that have been implicated in, for example, a cellular proliferation or immune disorder can be used as a “read out” or markers of the immune responsiveness of a particular cell.

For example, and not by way of limitation, genes, including PTMAX, that are modulated in cells by treatment with an agent (e.g., compound, drug or small molecule) that modulates PTMAX activity (e.g., identified in a screening assay as described herein) can be identified. Thus, to study the effect of agents on cellular proliferation disorders, for example, in a clinical trial, cells can be isolated and RNA prepared and analyzed for the levels of expression of PTMAX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of PTMAX or other genes. In this way, the gene expression pattern can serve as a marker, indicative of the physiological response of the cells to the agent. Accordingly, this response state may be determined before, and at various points during, treatment of the individual with the agent.

In one embodiment, the present invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of an PTMAX protein, mRNA, or genomic DNA in the pre-administration sample; (iii) obtaining one or more post-administration samples from the subject; (iv) detecting the level of expression or activity of the PTMAX protein, mRNA, or genomic DNA in the post-administration samples; (v) comparing the level of expression or activity of the PTMAX protein, mRNA, or genomic DNA in the pre-administration sample with the PTMAX protein, mRNA, or genomic DNA in the post administration sample or samples; and (vi) altering the administration of the agent to the subject accordingly. For example, increased administration of the agent may be desirable to increase the expression or activity of PTMAX to higher levels than detected, i.e., to increase the effectiveness of the agent. Alternatively, decreased administration of the agent may be desirable to decrease expression or activity of PTMAX to lower levels than detected, i.e., to decrease the effectiveness of the agent.

### Methods of Treatment

The present invention provides for both prophylactic and therapeutic methods of treating a subject at risk of (or susceptible to) a disorder or having a disorder associated with aberrant PTMAX expression or activity. For example, PTMA 1-6, 9 and 10 will be useful for both prophylactic and therapeutic methods of treating various cancers, viral diseases, and immune deficiency disorders. As a further example, PTMA 7 will be useful for both prophylactic and therapeutic methods of treating various cancers, coronary artery disease, arthritis, diabetic retinopathy, autoimmune diseases, and immune deficiency disorders. As a further example, PTMA 8 will be useful for both prophylactic and therapeutic methods of treating neurological diseases, psychiatric disorders, and inflammatory diseases.

### Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to, (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989, Science 244: 1288-1292); or (v) modulators ( i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, etc.).

### Prophylactic Methods

In one aspect, the invention provides a method for preventing, in a subject, a disease or condition associated with an aberrant PTMAX expression or activity, by administering to the subject an agent that modulates PTMAX expression or at least one PTMAX activity. Subjects at risk for a disease that is caused or contributed to by aberrant PTMAX expression or activity can be identified by, for example, any or a combination of diagnostic or prognostic assays as described herein. Administration of a prophylactic agent can occur prior to the manifestation of symptoms characteristic of the PTMAX aberrancy, such that a disease or disorder is prevented or, alternatively, delayed in its progression. Depending on the type of PTMAX aberrancy, for example, an PTMAX agonist or PTMAX antagonist agent can be used for treating the subject. The appropriate agent can be determined based on screening assays described herein. The prophylactic methods of the present invention are further discussed in the following subsections.

### Therapeutic Methods

Another aspect of the invention pertains to methods of modulating PTMAX expression or activity for therapeutic purposes. The modulatory method of the invention involves contacting a cell with an agent that modulates one or more of the activities of PTMAX protein activity associated with the cell. An agent that modulates PTMAX protein activity can be an agent as described herein, such as a nucleic acid or a protein, a naturally-occurring cognate ligand of an PTMAX protein, a peptide, an PTMAX peptidomimetic, or other small molecule. In one embodiment, the agent stimulates one or more PTMAX protein activity. Examples of such stimulatory agents include active PTMAX protein and a nucleic acid molecule encoding PTMAX that has been introduced into the cell. In another embodiment, the agent inhibits one or more PTMAX protein activity. Examples of such inhibitory agents include antisense PTMAX nucleic acid molecules and anti-PTMAX antibodies. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant expression or activity of a PTMAX protein or nucleic acid molecule. In one embodiment, the method involves administering an agent (e.g., an agent identified by a screening assay described herein), or combination of agents that modulates (e.g., upregulates or downregulates) PTMAX expression or activity. In another embodiment, the method involves administering an PTMAX protein or nucleic acid molecule as therapy to compensate for reduced or aberrant PTMAX expression or activity.

Stimulation of PTMAX activity is desirable in situations in which PTMAX is abnormally downregulated and/or in which increased PTMAX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

### Determination of the Biological Effect of the Therapeutic

In various embodiments of the present invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

### Malignancies

Therapeutics of the present invention may be useful in the therapeutic or prophylactic treatment of diseases or disorders that are associated with cell hyperproliferation and/or loss of control of cell proliferation (e.g., cancers, malignancies and tumors). For a review of such hyperproliferation disorders, see e.g., Fishman, et al., 1985. MEDICINE, 2nd ed., J. B. Lippincott Co., Philadelphia, Pa.

Therapeutics of the present invention may be assayed by any method known within the art for efficacy in treating or preventing malignancies and related disorders. Such assays include, but are not limited to, in vitro assays utilizing transformed cells or cells derived from the patient's tumor, as well as in vivo assays using animal models of cancer or malignancies. Potentially effective Therapeutics are those that, for example, inhibit the proliferation of tumor-derived or transformed cells in culture or cause a regression of tumors in animal models, in comparison to the controls.

In the practice of the present invention, once a malignancy or cancer has been shown to be amenable to treatment by modulating (i.e., inhibiting, antagonizing or agonizing) activity, that cancer or malignancy may subsequently be treated or prevented by the administration of a Therapeutic that serves to modulate protein function.

### Premalignant Conditions

The Therapeutics of the present invention that are effective in the therapeutic or prophylactic treatment of cancer or malignancies may also be administered for the treatment of pre-malignant conditions and/or to prevent the progression of a pre-malignancy to a neoplastic or malignant state. Such prophylactic or therapeutic use is indicated in conditions known or suspected of preceding progression to neoplasia or cancer, in particular, where non-neoplastic cell growth consisting of hyperplasia, metaplasia or, most particularly, dysplasia has occurred. For a review of such abnormal cell growth see e.g., Robbins & Angell, 1976. BASIC PATHOLOGY, 2nd ed., W.B. Saunders Co., Philadelphia, Pa.

Hyperplasia is a form of controlled cell proliferation involving an increase in cell number in a tissue or organ, without significant alteration in its structure or function. For example, it has been demonstrated that endometrial hyperplasia often precedes endometrial cancer. Metaplasia is a form of controlled cell growth in which one type of mature or fully differentiated cell substitutes for another type of mature cell. Metaplasia may occur in epithelial or connective tissue cells. Dysplasia is generally considered a precursor of cancer, and is found mainly in the epithelia. Dysplasia is the most disorderly form of non-neoplastic cell growth, and involves a loss in individual cell uniformity and in the architectural orientation of cells. Dysplasia characteristically occurs where there exists chronic irritation or inflammation, and is often found in the cervix, respiratory passages, oral cavity, and gall bladder.

Alternatively, or in addition to the presence of abnormal cell growth characterized as hyperplasia, metaplasia, or dysplasia, the presence of one or more characteristics of a transformed or malignant phenotype displayed either in vivo or in vitro within a cell sample derived from a patient, is indicative of the desirability of prophylactic/therapeutic administration of a Therapeutic that possesses the ability to modulate activity of An aforementioned protein. Characteristics of a transformed phenotype include, but are not limited to: (i) morphological changes; (ii) looser substratum attachment; (iii) loss of cell-to-cell contact inhibition; (iv) loss of anchorage dependence; (v) protease release; (vi) increased sugar transport; (vii) decreased serum requirement; (viii) expression of fetal antigens, (ix) disappearance of the 250 kDal cell-surface protein, and the like. See e.g., Richards, et al., 1986. MOLECULAR PATHOLOGY, W.B. Saunders Co., Philadelphia, Pa.

In a specific embodiment of the present invention, a patient that exhibits one or more of the following predisposing factors for malignancy is treated by administration of an effective amount of a Therapeutic: (i) a chromosomal translocation associated with a malignancy (e.g. the Philadelphia chromosome (bcr/abl) for chronic myelogenous leukemia and t(14; 18) for follicular lymphoma, etc.); (ii) familial polyposis or Gardner's syndrome (possible forerunners of colon cancer); (iii) monoclonal gammopathy of undetermined significance (a possible precursor of multiple myeloma) and (iv) a first degree kinship with persons having a cancer or pre-cancerous disease showing a Mendelian (genetic) inheritance pattern (e.g., familial polyposis of the colon, Gardner's syndrome, hereditary exostosis, polyendocrine adenomatosis, Peutz-Jeghers syndrome, neurofibromatosis of Von Recklinghausen, medullary thyroid carcinoma with amyloid production and pheochromocytoma, retinoblastoma, carotid body tumor, cutaneous melanocarcinoma, intraocular melanocarcinoma, xeroderma pigmentosum, ataxia telangiectasia, Chediak-Higashi syndrome, albinism, Fanconi's aplastic anemia and Bloom's syndrome).

In another embodiment, a Therapeutic of the present invention is administered to a human patient to prevent the progression to breast, colon, lung, pancreatic, or uterine cancer, or melanoma or sarcoma.

### Hyperproliferative and Dysproliferative Disorders

In one embodiment of the present invention, a Therapeutic is administered in the therapeutic or prophylactic treatment of hyperproliferative or benign dysproliferative disorders. The efficacy in treating or preventing hyperproliferative diseases or disorders of a Therapeutic of the present invention may be assayed by any method known within the art. Such assays include in vitro cell proliferation assays, in vitro or in vivo assays using animal models of hyperproliferative diseases or disorders, or the like. Potentially effective Therapeutics may, for example, promote cell proliferation in culture or cause growth or cell proliferation in animal models in comparison to controls.

Specific embodiments of the present invention are directed to the treatment or prevention of cirrhosis of the liver (a condition in which scarring has overtaken normal liver regeneration processes); treatment of keloid (hypertrophic scar) formation causing disfiguring of the skin in which the scarring process interferes with normal renewal; psoriasis (a common skin condition characterized by excessive proliferation of the skin and delay in proper cell fate determination); benign tumors; fibrocystic conditions and tissue hypertrophy (e.g., benign prostatic hypertrophy).

### Neurodegenerative Disorders

PTMAX protein have been implicated in the deregulation of cellular maturation and apoptosis, which are both characteristic of neurodegenerative disease. Accordingly, Therapeutics of the invention, particularly but not limited to those that modulate (or supply) activity of an aforementioned protein, may be effective in treating or preventing neurodegenerative disease. Therapeutics of the present invention that modulate the activity of an aforementioned protein involved in neurodegenerative disorders can be assayed by any method known in the art for efficacy in treating or preventing such neurodegenerative diseases and disorders. Such assays include in vitro assays for regulated cell maturation or inhibition of apoptosis or in vivo assays using animal models of neurodegenerative diseases or disorders, or any of the assays described below. Potentially effective Therapeutics, for example but not by way of limitation, promote regulated cell maturation and prevent cell apoptosis in culture, or reduce neurodegeneration in animal models in comparison to controls.

Once a neurodegenerative disease or disorder has been shown to be amenable to treatment by modulation activity, that neurodegenerative disease or disorder can be treated or prevented by administration of a Therapeutic that modulates activity. Such diseases include all degenerative disorders involved with aging, especially osteoarthritis and neurodegenerative disorders.

### Disorders Related to Organ Transplantation

PTMAX has been implicated in disorders related to organ transplantation, in particular but not limited to organ rejection. Therapeutics of the invention, particularly those that modulate (or supply) activity, may be effective in treating or preventing diseases or disorders related to organ transplantation. Therapeutics of the invention (particularly Therapeutics that modulate the levels or activity of an aforementioned protein) can be assayed by any method known in the art for efficacy in treating or preventing such diseases and disorders related to organ transplantation. Such assays include in vitro assays for using cell culture models as described below, or in vivo assays using animal models of diseases and disorders related to organ transplantation, see e.g., below. Potentially effective Therapeutics, for example but not by way of limitation, reduce immune rejection responses in animal models in comparison to controls.

Accordingly, once diseases and disorders related to organ transplantation are shown to be amenable to treatment by modulation of activity, such diseases or disorders can be treated or prevented by administration of a Therapeutic that modulates activity.

### Cytokine and Cell Proliferation/Differentiation Activity

A PTMAX protein of the present invention may exhibit cytokine, cell proliferation (either inducing or inhibiting) or cell differentiation (either inducing or inhibiting) activity or may induce production of other cytokines in certain cell populations. Many protein factors discovered to date, including all known cytokines, have exhibited activity in one or more factor dependent cell proliferation assays, and hence the assays serve as a convenient confirmation of cytokine activity. The activity of a protein of the present invention is evidenced by any one of a number of routine factor dependent cell proliferation assays for cell lines including, without limitation, 32D, DA2, DA1G, T10, B9, B9/11, BaF3, MC9/G, M+(preB M+), 2E8, RB5, DA1, 123, T1165, HT2, CTLL2, TF-1, Mo7e and CMK.

The activity of a protein of the invention may, among other means, be measured by the following methods: Assays for T-cell or thymocyte proliferation include without limitation those described in: CURRENT PROTOCOLS IN IMMUNOLOGY, Ed by Coligan et al., Greene Publishing Associates and Wiley-Interscience (Chapter 3 and Chapter 7); Takai et al., J Immunol 137:3494-3500, 1986; Bertagnolli et al., J Immunol 145:1706-1712, 1990; Bertagnolli et al., Cell Immunol 133:327-341, 1991; Bertagnolli, et al., J Immunol 149:3778-3783, 1992; Bowman et al., J Immunol 152:1756-1761, 1994.

Assays for cytokine production and/or proliferation of spleen cells, lymph node cells or thymocytes include, without limitation, those described by Kruisbeek and Shevach, In: CURRENT PROTOCOLS IN IMMUNOLOGY. Coligan et al., eds. Vol 1, pp. 3.12.1-14, John Wiley and Sons, Toronto 1994; and by Schreiber, In: CURRENT PROTOCOLS IN IMMUNOLOGY. Coligan eds. Vol 1 pp. 6.8.1-8, John Wiley and Sons, Toronto 1994.

Assays for proliferation and differentiation of hematopoietic and lymphopoietic cells include, without limitation, those described by Bottomly et al., In: CURRENT PROTOCOLS IN IMMUNOLOGY. Coligan et al., eds. Vol 1 pp. 6.3.1-6.3.12, John Wiley and Sons, Toronto 1991; deVries et al., J Exp Med 173:1205-1211, 1991; Moreau et al., Nature 336:690-692, 1988; Greenberger et al., Proc Natl Acad Sci U.S.A. 80:2931-2938, 1983; Nordan, In: CURRENT PROTOCOLS IN IMMUNOLOGY. Coligan et al., eds. Vol 1 pp. 6.6.1-5, John Wiley and Sons, Toronto 1991; Smith et al., Proc Natl Acad Sci U.S.A. 83:1857-1861, 1986; Measurement of human Interleukin 11-Bennett, et al. In: CURRENT PROTOCOLS IN IMMUNOLOGY. Coligan et al., eds. Vol 1 pp. 6.15.1 John Wiley and Sons, Toronto 1991; Ciarletta, et al., In: CURRENT PROTOCOLS IN IMMUNOLOGY. Coligan et al., eds. Vol 1 pp. 6.13.1, John Wiley and Sons, Toronto 1991.

Assays for T-cell clone responses to antigens (which will identify, among others, proteins that affect APC-T cell interactions as well as direct T-cell effects by measuring proliferation and cytokine production) include, without limitation, those described In: CURRENT PROTOCOLS IN IMMUNOLOGY. Coligan et al., eds., Greene Publishing Associates and Wiley-Interscience (Chapter 3Chapter 6, Chapter 7); Weinberger et al., Proc Natl Acad Sci USA 77:6091-6095, 1980; Weinberger et al., Eur J Immun 11:405-411, 1981; Takai et al., J Immunol 137:3494-3500, 1986; Takai et al., J Immunol 140:508-512, 1988.

### Immune Stimulating or Suppressing Activity

A PTMAX protein of the present invention may also exhibit immune stimulating or immune suppressing activity, including without limitation the activities for which assays are described herein. A protein may be useful in the treatment of various immune deficiencies and disorders (including severe combined immunodeficiency (SCID)), e.g., in regulating (up or down) growth and proliferation of T and/or B lymphocytes, as well as effecting the cytolytic activity of NK cells and other cell populations. These immune deficiencies may be genetic or be caused by vital (e.g., HIV) as well as bacterial or fungal infections, or may result from autoimmune disorders. More specifically, infectious diseases causes by vital, bacterial, fungal or other infection may be treatable using a protein of the present invention, including infections by HIV, hepatitis viruses, herpesviruses, mycobacteria, Leishmania species., malaria species. and various fungal infections such as candidiasis. Of course, in this regard, a protein of the present invention may also be useful where a boost to the immune system generally may be desirable, i.e., in the treatment of cancer.

Autoimmune disorders which may be treated using a protein of the present invention include, for example, connective tissue disease, multiple sclerosis, systemic lupus erythematosus, rheumatoid arthritis, autoimmune pulmonary inflammation, Guillain-Barre syndrome, autoimmune thyroiditis, insulin dependent diabetes mellitus, myasthenia gravis, graft-versus-host disease and autoimmune inflammatory eye disease. Such a protein of the present invention may also to be useful in the treatment of allergic reactions and conditions, such as asthma (particularly allergic asthma) or other respiratory problems. Other conditions, in which immune suppression is desired (including, for example, organ transplantation), may also be treatable using a protein of the present invention.

Using the proteins of the invention it may also be possible to immune responses, in a number of ways. Down regulation may be in the form of inhibiting or blocking an immune response already in progress or may involve preventing the induction of an immune response. The functions of activated T cells may be inhibited by suppressing T cell responses or by inducing specific tolerance in T cells, or both. Immunosuppression of T cell responses is generally an active, non-antigen-specific, process which requires continuous exposure of the T cells to the suppressive agent. Tolerance, which involves inducing non-responsiveness or energy in T cells, is distinguishable from immunosuppression in that it is generally antigen-specific and persists after exposure to the tolerizing agent has ceased. Operationally, tolerance can be demonstrated by the lack of a T cell response upon re-exposure to specific antigen in the absence of the tolerizing agent.

Down regulating or preventing one or more antigen functions (including without limitation B lymphocyte antigen functions (such as, for example, B7), e.g., preventing high level lymphokine synthesis by activated T cells, will be useful in situations of tissue, skin and organ transplantation and in graft-versus-host disease (GVHD). For example, blockage of T cell function should result in reduced tissue destruction in tissue transplantation. Typically, in tissue transplants, rejection of the transplant is initiated through its recognition as foreign by T cells, followed by an immune reaction that destroys the transplant. The administration of a molecule which inhibits or blocks interaction of a B7 lymphocyte antigen with its natural ligand(s) on immune cells (such as a soluble, monomeric form of a peptide having B7-2 activity alone or in conjunction with a monomeric form of a peptide having an activity of another B lymphocyte antigen (e.g., B7-1, B7-3) or blocking antibody), prior to transplantation can lead to the binding of the molecule to the natural ligand(s) on the immune cells without transmitting the corresponding costimulatory signal. Blocking B lymphocyte antigen function in this matter prevents cytokine synthesis by immune cells, such as T cells, and thus acts as an immunosuppressant. Moreover, the lack of costimulation may also be sufficient to energize the T cells, thereby inducing tolerance in a subject. Induction of long-term tolerance by B lymphocyte antigen-blocking reagents may avoid the necessity of repeated administration of these blocking reagents. To achieve sufficient immunosuppression or tolerance in a subject, it may also be necessary to block the function of B lymphocyte antigens.

The efficacy of particular blocking reagents in preventing organ transplant rejection or GVHD can be assessed using animal models that are predictive of efficacy in humans. Examples of appropriate systems which can be used include allogeneic cardiac grafts in rats and xenogeneic pancreatic islet cell grafts in mice, both of which have been used to examine the immunosuppressive effects of CTLA4Ig fusion proteins in vivo as described in Lenschow et al., Science 257:789-792 (1992) and Turka et al., Proc Natl Acad Sci USA, 89:11102-11105 (1992). In addition, murine models of GVHD (see Paul ed., FUNDAMENTAL IMMUNOLOGY, Raven Press, New York, 1989, pp. 846-847) can be used to determine the effect of blocking B lymphocyte antigen function in vivo on the development of that disease.

Blocking antigen function may also be therapeutically useful for treating autoimmune diseases. Many autoimmune disorders are the result of inappropriate activation of T cells that are reactive against self tissue and which promote the production of cytokines and auto-antibodies involved in the pathology of the diseases. Preventing the activation of autoreactive T cells may reduce or eliminate disease symptoms. Administration of reagents which block costimulation of T cells by disrupting receptor:ligand interactions of B lymphocyte antigens can be used to inhibit T cell activation and prevent production of auto-antibodies or T cell-derived cytokines which may be involved in the disease process. Additionally, blocking reagents may induce antigen-specific tolerance of autoreactive T cells which could lead to long-term relief from the disease. The efficacy of blocking reagents in preventing or alleviating autoimmune disorders can be determined using a number of well-characterized animal models of human autoimmune diseases. Examples include murine experimental autoimmune encephalitis, systemic lupus erythematosis in MRL/1pr/1pr mice or NZB hybrid mice, murine autoimmune collagen arthritis, diabetes mellitus in NOD mice and BB rats, and murine experimental myasthenia gravis (see Paul ed., FUNDAMENTAL IMMUNOLOGY, Raven Press, New York, 1989, pp. 840-856).

Upregulation of an antigen function (preferably a B lymphocyte antigen function), as a means of up regulating immune responses, may also be useful in therapy. Upregulation of immune responses may be in the form of enhancing an existing immune response or eliciting an initial immune response. For example, enhancing an immune response through stimulating B lymphocyte antigen function may be useful in cases of viral infection. In addition, systemic vital diseases such as influenza, the common cold, and encephalitis might be alleviated by the administration of stimulatory forms of B lymphocyte antigens systemically.

Alternatively, anti-viral immune responses may be enhanced in an infected patient by removing T cells from the patient, costimulating the T cells in vitro with viral antigen-pulsed APCs either expressing a peptide of the present invention or together with a stimulatory form of a soluble peptide of the present invention and reintroducing the in vitro activated T cells into the patient. Another method of enhancing anti-vital immune responses would be to isolate infected cells from a patient, transfect them with a nucleic acid encoding a protein of the present invention as described herein such that the cells express all or a portion of the protein on their surface, and reintroduce the transfected cells into the patient. The infected cells would now be capable of delivering a costimulatory signal to, and thereby activate, T cells in vivo.

In another application, up regulation or enhancement of antigen function (preferably B lymphocyte antigen function) may be useful in the induction of tumor immunity. Tumor cells (e.g., sarcoma, melanoma, lymphoma, leukemia, neuroblastoma, carcinoma) transfected with a nucleic acid encoding at least one peptide of the present invention can be administered to a subject to overcome tumor-specific tolerance in the subject. If desired, the tumor cell can be transfected to express a combination of peptides. For example, tumor cells obtained from a patient can be transfected ex vivo with an expression vector directing the expression of a peptide having B7-2-like activity alone, or in conjunction with a peptide having B7-1-like activity and/or B7-3-like activity. The transfected tumor cells are returned to the patient to result in expression of the peptides on the surface of the transfected cell. Alternatively, gene therapy techniques can be used to target a tumor cell for transfection in vivo.

The presence of the peptide of the present invention having the activity of a B lymphocyte antigen(s) on the surface of the tumor cell provides the necessary costimulation signal to T cells to induce a T cell mediated immune response against the transfected tumor cells. In addition, tumor cells which lack MHC class I or MHC class II molecules, or which fail to reexpress sufficient amounts of MHC class I or MHC class II molecules, can be transfected with nucleic acid encoding all or a portion of (e.g., a cytoplasmic-domain truncated portion) of an MHC class I □ chain protein and □₂ microglobulin protein or an MHC class II a chain protein and an MHC class II □ chain protein to thereby express MHC class I or MHC class II proteins on the cell surface. Expression of the appropriate class I or class II MHC in conjunction with a peptide having the activity of a B lymphocyte antigen (e.g., B7-1, B7-2, B7-3) induces a T cell mediated immune response against the transfected tumor cell. Optionally, a gene encoding an antisense construct which blocks expression of an MHC class II associated protein, such as the invariant chain, can also be cotransfected with a DNA encoding a peptide having the activity of a B lymphocyte antigen to promote presentation of tumor associated antigens and induce tumor specific immunity. Thus, the induction of a T cell mediated immune response in a human subject may be sufficient to overcome tumor-specific tolerance in the subject.

The activity of a protein of the invention may, among other means, be measured by the following methods: Suitable assays for thymocyte or splenocyte cytotoxicity include, without limitation, those described In: CURRENT PROTOCOLS IN IMMUNOLOGY. Coligan et al., eds. Greene Publishing Associates and Wiley-Interscience (Chapter 3, Chapter 7); Herrmann et al., Proc Natl Acad Sci USA 78:2488-2492, 1981; Herrmann et al., J Immunol 128:1968-1974, 1982; Handa et al., J Immunol 135:1564-1572, 1985; Takai et al., J Immunol 137:3494-3500, 1986; Takai et al., J Immunol 140:508-512, 1988; Herrmann et al., Proc Natl Acad Sci USA 78:2488-2492, 1981; Herrmann et al., J Immunol 128:1968-1974, 1982; Handa et al., J Immunol 135:1564-1572, 1985; Takai et al., J Immunol 137:3494-3500, 1986; Bowman et al., J Virology 61:1992-1998; Takai et al., J Immunol 140:508-512, 1988; Bertagnolli et al., Cell Immunol 133:327-341, 1991; Brown et al., J Immunol 153:3079-3092, 1994.

Assays for T-cell-dependent immunoglobulin responses and isotype switching (which will identify, among others, proteins that modulate T-cell dependent antibody responses and that affect Th1/Th2 profiles) include, without limitation, those described in: Maliszewski, J Immunol 144:3028-3033, 1990; and Mond and Brunswick In: CURRENT PROTOCOLS IN IMMUNOLOGY. Coligan et al., (eds.) Vol 1 pp. 3.8.1-3.8.16, John Wiley and Sons, Toronto 1994.

Mixed lymphocyte reaction (MLR) assays (which will identify, among others, proteins that generate predominantly Th1 and CTL responses) include, without limitation, those described In: CURRENT PROTOCOLS IN IMMUNOLOGY. Coligan et al., eds. Greene Publishing Associates and Wiley-Interscience (Chapter 3, Chapter 7); Takai et al., J Immunol 137:3494-3500, 1986; Takai et al., J Immunol 140:508-512, 1988; Bertagnolli et al., J Immunol 149:3778-3783, 1992.

Dendritic cell-dependent assays (which will identify, among others, proteins expressed by dendritic cells that activate naive T-cells) include, without limitation, those described in: Guery et al., J Immunol 134:536-544, 1995; Inaba et al., J Exp Med 173:549-559, 1991; Macatonia et al., J Immunol 154:5071-5079, 1995; Porgador et al., J Exp Med 182:255-260, 1995; Nair et al., J Virol 67:4062-4069, 1993; Huang et al., Science 264:961-965, 1994; Macatonia et al., J Exp Med 169:1255-1264, 1989; Bhardwaj et al., J Clin Investig 94:797-807, 1994; and Inaba et al., J Exp Med 172:631-640, 1990.

Assays for lymphocyte survival/apoptosis (which will identify, among others, proteins that prevent apoptosis after superantigen induction and proteins that regulate lymphocyte homeostasis) include, without limitation, those described in: Darzynkiewicz et al., Cytometry 13:795-808, 1992; Gorczyca et al., Leukemia 7:659-670, 1993; Gorczyca et al., Cancer Res 53:1945-1951, 1993; Itoh et al., Cell 66:233-243, 1991; Zacharchuk, J Immunol 145:4037-4045, 1990; Zamai et al., Cytometry 14:891-897, 1993; Gorczyca et al., Internat J Oncol 1:639-648, 1992.

Assays for proteins that influence early steps of T-cell commitment and development include, without limitation, those described in: Antica et al., Blood 84:111-117, 1994; Fine et al., Cell Immunol 155: 111-122, 1994; Galy et al., Blood 85:2770-2778, 1995; Toki et al., Proc Nat AcadSci USA 88:7548-7551, 1991.

### Hematopoiesis Regulating Activity

A PTMAX protein of the present invention may be useful in regulation of hematopoiesis and, consequently, in the treatment of myeloid or lymphoid cell deficiencies. Even marginal biological activity in support of colony forming cells or of factor-dependent cell lines indicates involvement in regulating hematopoiesis, e.g. in supporting the growth and proliferation of erythroid progenitor cells alone or in combination with other cytokines, thereby indicating utility, for example, in treating various anemias or for use in conjunction with irradiation/chemotherapy to stimulate the production of erythroid precursors and/or erythroid cells; in supporting the growth and proliferation of myeloid cells such as granulocytes and monocytes/macrophages (i.e., traditional CSF activity) useful, for example, in conjunction with chemotherapy to prevent or treat consequent myelo-suppression; in supporting the growth and proliferation of megakaryocytes and consequently of platelets thereby allowing prevention or treatment of various platelet disorders such as thrombocytopenia, and generally for use in place of or complimentary to platelet transfusions; and/or in supporting the growth and proliferation of hematopoietic stem cells which are capable of maturing to any and all of the above-mentioned hematopoietic cells and therefore find therapeutic utility in various stem cell disorders (such as those usually treated with transplantation, including, without limitation, aplastic anemia and paroxysmal nocturnal hemoglobinuria), as well as in repopulating the stem cell compartment post irradiation/chemotherapy, either in-vivo or ex-vivo (i.e., in conjunction with bone marrow transplantation or with peripheral progenitor cell transplantation (homologous or heterologous)) as normal cells or genetically manipulated for gene therapy.

The activity of a protein of the invention may, among other means, be measured by the following methods:

Suitable assays for proliferation and differentiation of various hematopoietic lines are cited above.

Assays for embryonic stem cell differentiation (which will identify, among others, proteins that influence embryonic differentiation hematopoiesis) include, without limitation, those described in: Johansson et al. Cellular Biology 15:141-151, 1995; Keller et al., Mol. Cell. Biol. 13:473-486, 1993; McClanahan et al., Blood 81:2903-2915, 1993.

Assays for stem cell survival and differentiation (which will identify, among others, proteins that regulate lympho-hematopoiesis) include, without limitation, those described in: Methylcellulose colony forming assays, Freshney, In: CULTURE OF HEMATOPOIETIC CELLS. Freshney, et al. (eds.) Vol pp. 265-268, Wiley-Liss, Inc., New York, N.Y. 1994; Hirayama et al., Proc Natl Acad Sci USA 89:5907-5911, 1992; McNiece and Briddeli, In: CULTURE OF HEMATOPOIETIC CELLS. Freshney, et al. (eds.) Vol pp. 23-39, Wiley-Liss, Inc., New York, N.Y. 1994; Neben et al., Exp Hematol 22:353-359, 1994; Ploemacher, In: CULTURE OF HEMATOPOIETIC CELLS. Freshney, et al eds. Vol pp. 1-21, Wiley-Liss, Inc., New York, N.Y. 1994; Spooncer et al.,

In: CULTURE OF HEMATOPOIETIC CELLS. Freshhey, et al., (eds.) Vol pp. 163-179, Wiley-Liss, Inc., New York, N.Y. 1994; Sutherland, In: CULTURE OF HEMATOPOIETIC CELLS. Freshney, et al., (eds.) Vol pp. 139-162, Wiley-Liss, Inc., New York, N.Y. 1994.

### Tissue Growth Activity

A PTMAX protein of the present invention also may have utility in compositions used for nerve tissue growth or regeneration, as well as for wound healing and tissue repair and replacement.

The protein of the present invention may also be useful for proliferation of neural cells and for regeneration of nerve and brain tissue, i.e. for the treatment of central and peripheral nervous system diseases and neuropathies, as well as mechanical and traumatic disorders, which involve degeneration, death or trauma to neural cells or nerve tissue. More specifically, a protein may be used in the treatment of diseases of the peripheral nervous system, such as peripheral nerve injuries, peripheral neuropathy and localized neuropathies, and central nervous system diseases, such as Alzheimer's, Parkinson's disease, Huntington's disease, amyotrophic lateral sclerosis, and Shy-Drager syndrome. Further conditions which may be treated in accordance with the present invention include mechanical and traumatic disorders, such as spinal cord disorders, head trauma and cerebrovascular diseases such as stroke. Peripheral neuropathies resulting from chemotherapy or other medical therapies may also be treatable using a protein of the invention.

Proteins of the invention may also be useful to promote better or faster closure of non-healing wounds, including without limitation pressure ulcers, ulcers associated with vascular insufficiency, surgical and traumatic wounds, and the like.

It is expected that a protein of the present invention may also exhibit activity for generation or regeneration of other tissues, such as organs (including, for example, pancreas, liver, intestine, kidney, skin, endothelium), muscle (smooth, skeletal or cardiac) and vascular (including vascular endothelium) tissue, or for promoting the growth of cells comprising such tissues. Part of the desired effects may be by inhibition or modulation of fibrotic scarring to allow normal tissue to regenerate. A protein of the invention may also exhibit angiogenic activity.

A protein of the present invention may also be useful for gut protection or regeneration and treatment of lung or liver fibrosis, reperfusion injury in various tissues, and conditions resulting from systemic cytokine damage.

A protein of the present invention may also be useful for promoting or inhibiting differentiation of tissues described above from precursor tissues or cells; or for inhibiting the growth of tissues described above.

The activity of a protein of the invention may, among other means, be measured by the following methods:

Assays for tissue generation activity include, without limitation, those described in: International Patent Publication No. WO95/16035 (bone, cartilage, tendon); International Patent Publication No. WO95/05846 (nerve, neuronal); International Patent Publication No. WO91/07491 (skin, endothelium).

Assays for wound healing activity include, without limitation, those described in: Winter, EPIDERMAL WOUND HEALING, pp. 71-112 (Maibach and Rovee, eds.), Year Book Medical Publishers, Inc., Chicago, as modified by Eaglstein and Menz, J. Invest. Dermatol 71:382-84 (1978).

### Chemotactic/Chemokinetic Activity

A protein of the present invention may have chemotactic or chemokinetic activity (e.g., act as a chemokine) for mammalian cells, including, for example, monocytes, fibroblasts, neutrophils, T-cells, mast cells, eosinophils, epithelial and/or endothelial cells. Chemotactic and chemokinetic proteins can be used to mobilize or attract a desired cell population to a desired site of action. Chemotactic or chemokinetic proteins provide particular advantages in treatment of wounds and other trauma to tissues, as well as in treatment of localized infections. For example, attraction of lymphocytes, monocytes or neutrophils to tumors or sites of infection may result in improved immune responses against the tumor or infecting agent.

A protein or peptide has chemotactic activity for a particular cell population if it can stimulate, directly or indirectly, the directed orientation or movement of such cell population. Preferably, the protein or peptide has the ability to directly stimulate directed movement of cells. Whether a particular protein has chemotactic activity for a population of cells can be readily determined by employing such protein or peptide in any known assay for cell chemotaxis.

The activity of a protein of the invention may, among other means, be measured by following methods:

Assays for chemotactic activity (which will identify proteins that induce or prevent chemotaxis) consist of assays that measure the ability of a protein to induce the migration of cells across a membrane as well as the ability of a protein to induce the adhesion of one cell population to another cell population. Suitable assays for movement and adhesion include, without limitation, those described in: CURRENT PROTOCOLS IN IMMUNOLOGY, Coligan et al., eds. (Chapter 6.12, MEASUREMENT OF ALPHA AND BETA CHEMOKINES 6.12.1-6.12.28); Taub et al. J Clin Invest 95:1370-1376, 1995; Lind et al. APMIS 103:140-146, 1995; Muller et al., Eur J Immunol 25: 1744-1748; Gruberet al. J Immunol 152:5860-5867, 1994; Johnston et al., J Immunol 153: 1762-1768, 1994.

### Receptor/Ligand Activity

A protein of the present invention may also demonstrate activity as receptors, receptor ligands or inhibitors or agonists of receptor/ligand interactions. Examples of such receptors and ligands include, without limitation, cytokine receptors and their ligands, receptor kinases and their ligands, receptor phosphatases and their ligands, receptors involved in cell—cell interactions and their ligands (including without limitation, cellular adhesion molecules (such as selecting, integrins and their ligands) and receptor/ligand pairs involved in antigen presentation, antigen recognition and development of cellular and humoral immune responses). Receptors and ligands are also useful for screening of potential peptide or small molecule inhibitors of the relevant receptor/ligand interaction. A protein of the present invention (including, without limitation, fragments of receptors and ligands) may themselves be useful as inhibitors of receptor/ligand interactions.

The activity of a protein of the invention may, among other means, be measured by the following methods:

Suitable assays for receptor-ligand activity include without limitation those described in: CURRENT PROTOCOLS IN IMMUNOLOGY, Ed by Coligan, et al., Greene Publishing Associates and Wiley-Interscience (Chapter 7.28, Measurement of Cellular Adhesion under static conditions 7.28.1-7.28.22), Takai et al., Proc Natl Acad Sci USA 84:6864-6868, 1987; Bierer et al., J. Exp. Med. 168:1145-1156, 1988; Rosenstein et al., J. Exp. Med. 169:149-160 1989; Stoltenborg et al., J Immunol Methods 175:59-68, 1994; Stitt et al., Cell 80:661-670, 1995.

### Anti-Inflammatory Activity

Proteins of the present invention may also exhibit anti-inflammatory activity. The anti-inflammatory activity may be achieved by providing a stimulus to cells involved in the inflammatory response, by inhibiting or promoting cell—cell interactions (such as, for example, cell adhesion), by inhibiting or promoting chemotaxis of cells involved in the inflammatory process, inhibiting or promoting cell extravasation, or by stimulating or suppressing production of other factors which more directly inhibit or promote an inflammatory response. Proteins exhibiting such activities can be used to treat inflammatory conditions including chronic or acute conditions), including without limitation inflammation associated with infection (such as septic shock, sepsis or systemic inflammatory response syndrome (SIRS)), ischemia-reperfusion injury, endotoxin lethality, arthritis, complement-mediated hyperacute rejection, nephritis, cytokine or chemokine-induced lung injury, inflammatory bowel disease, Crohn's disease or resulting from over production of cytokines such as TNF or hypersensitivity to an antigenic substance or material.

### Tumor Inhibition Activity

In addition to the activities described above for immunological treatment or prevention of tumors, a protein of the invention may exhibit other anti-tumor activities. A protein may inhibit tumor growth directly or indirectly (such as, for example, via ADCC). A protein may exhibit its tumor inhibitory activity by acting on tumor tissue or tumor precursor tissue, by inhibiting formation of tissues necessary to support tumor growth (such as, for example, by inhibiting angiogenesis), by causing production of other factors, agents or cell types which inhibit tumor growth, or by suppressing, eliminating or inhibiting factors, agents or cell types which promote tumor growth.

### EXAMPLES

### Example 1

### Radiation Hybrid Mapping for Various Clones

### Radiation Hybrid Mapping Provides the Chromosomal Location of Clones

Radiation hybrid mapping using human chromosome markers was carried out for many of the clones described in the present invention. The procedure used to obtain these results is analogous to that described in Steen, R G et al. (A High-Density Integrated Genetic Linkage and Radiation Hybrid Map of the Laboratory Rat, Genome Research 1999 (Published Online on May 21, 1999)Vol. 9, AP1-AP8, 1999). A panel of 93 cell clones containing randomized radiation-induced human chromosomal fragments was screened in 96 well plates using PCR primers designed to identify the sought clones in a unique fashion. Table 3 provides the results obtained for clones AC010784-1 and AC010175_A.0.1.

TABLE 3
Chromosomal mapping from radiation hybrid results.

| | | Distance from | Distance from |
|---|---|---|---|
| Clone | Chromosome | Marker, cR | Marker, cR |
| AC010784-1 | 4 | WI-4767, 8.4 cR | WI-5565, 0.0 cR |
| AC010175_A.0.1 | 12 | D12S358, 4.2 cR | AFMA184ZC1, |
| | | | 2.5 cR |

### Example 2

### Quantitative Expression Analysis of PTMAX Nucleic Acids

The quantitative expression of various clones was assessed using microtiter plates containing RNA samples from a variety of normal and pathology-derived cells, cell lines and tissues using real time quantitative PCR (RTQ PCR). RTQ PCR was performed on a Perkin-Elmer Biosystems ABI PRISM® 7700 Sequence Detection System. Various collections of samples are assembled on the plates, and referred to as Panel 1 (containing normal tissues and cancer cell lines), Panel 2 (containing samples derived from tissues from normal and cancer sources), Panel 3 (containing cancer cell lines), Panel 4 (containing cells and cell lines from normal tissues and cells related to inflammatory conditions), Panel 5D/5I (containing human tissues and cell lines with an emphasis on metabolic diseases), Panel CNSD.01 (containing samples from normal and diseased brains) and CNS_neurodegeneration_panel (containing samples from normal and diseased brains).

RNA integrity from all samples is controlled for quality by visual assessment of agarose gel electropherograms using 28S and 18S ribosomal RNA staining intensity ratio as a guide (2:1 to 2.5:1 28s: 18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

First, the RNA samples were normalized to reference nucleic acids such as constitutively expressed genes (for example, β-actin and GAPDH). Normalized RNA (5 ul) was converted to cDNA and analyzed by RTQ-PCR using One Step RT-PCR Master Mix Reagents (PE Biosystems; Catalog No. 4309169) and gene-specific primers according to the manufacturer's instructions. Probes and primers were designed for each assay according to Perkin Elmer Biosystem's Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tₘ) range=58°−60° C., primer optimal Tm=59° C., maximum primer difference=2° C., probe does not have 5′ G, probe Tₘ must be 10° C. greater than primer Tₘ, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: Normalized RNA from each tissue and each cell line was spotted in each well of a 96 well PCR plate (Perkin Elmer Biosystems). PCR cocktails including two probes (a probe specific for the target clone and another gene-specific probe multiplexed with the target probe) were set up using 1×TaqMan™ PCR Master Mix for the PE Biosystems 7700, with 5 mM MgCl2, dNTPs (dA, G, C, U at 1:1:1:2 ratios), 0.25 U/ml AmpliTaq Gold™ (PE Biosystems), and 0.4 U/μl RNase inhibitor, and 0.25 U/μl reverse transcriptase. Reverse transcription was performed at 48° C. for 30 minutes followed by amplification/PCR cycles as follows: 95° C. 10 min, then 40 cycles of 95° C. for 15 seconds, 60° C. for 1 minute. Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100.

### Panels 1, 1.1, 1.2, and 1.3D

The plates for Panels 1, 1. 1, 1.2 and 1 .3D include 2 control wells (genomic DNA control and chemistry control) and 94 wells containing cDNA from various samples. The samples in these panels are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in these panels are widely available through the American Type Culture Collection (ATCC), a repository for cultured cell lines, and were cultured using the conditions recommended by the ATCC. The normal tissues found on these panels are comprised of samples derived from all major organ systems from single adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose.

In the results for Panels 1, 1. 1, 1.2 and 1 .3D, the following abbreviations are used:

ca.=carcinoma,

*=established from metastasis,

met=metastasis,

s cell var=small cell variant,

non-s=non-sm=non-small,

squam=squamous,

pl. eff=pl effusion=pleural effusion,

glio=glioma,

astro=astrocytoma, and

neuro=neuroblastoma.

### GENERAL_SCREENING_PANEL_V1.4

The plates for Panel 1.4 include 2 control wells (genomic DNA control and chemistry control) and 94 wells containing cDNA from various samples. The samples in Panel 1.4 are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in Panel 1.4 are widely available through the American Type Culture Collection (ATCC), a repository for cultured cell lines, and were cultured using the conditions recommended by the ATCC. The normal tissues found on Panel 1.4 are comprised of pools of samples derived from all major organ systems from 2 to 5 different adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose. See Panels 1, 1.1, 1.2 and 1.3D for description of abbreviations.

### Panels 2D and 2.2

The plates for Panels 2D and 2.2 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologists at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissues were ascertained to be free of disease and were purchased from various commercial sources such as Clontech (Palo Alto, Calif.), Research Genetics, and Invitrogen.

### Panel 3D

The plates of Panel 3D are comprised of 94 cDNA samples and two control samples. Specifically, 92 of these samples are derived from cultured human cancer cell lines, 2 samples of human primary cerebellar tissue and 2 controls. The human cell lines are generally obtained from ATCC (American Type Culture Collection), NCI or the German tumor cell bank and fall into the following tissue groups: Squamous cell carcinoma of the tongue, breast cancer, prostate cancer, melanoma, epidermoid carcinoma, sarcomas, bladder carcinomas, pancreatic cancers, kidney cancers, leukemias/lymphomas, ovarian/uterine/cervical, gastric, colon, lung and CNS cancer cell lines. In addition, there are two independent samples of cerebellum. These cells are all cultured under standard recommended conditions and RNA extracted using the standard procedures. The cell lines in panel 3D and 1.3D are of the most common cell lines used in the scientific literature.

### Panels 4D, 4R, and 4.1D

Panel 4 includes samples on a 96 well plate (2 control wells, 94 test samples) composed of RNA (Panel 4R) or cDNA (Panels 4D/4.1D) isolated from various human cell lines or tissues related to inflammatory conditions. Total RNA from control normal tissues such as colon and lung (Stratagene, La Jolla, Calif.) and thymus and kidney (Clontech) were employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5×10⁻⁵ M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50 ng/ml and IL-18 at 5-10 ng/ml for 6 hours. In some c mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶ cells/ml in DMEM 5% FCS (Hyclone), 100 μM non essential amino acids (Gibco), 1 mM sodium pyruvate (Gibco), mercaptoethanol (5.5×10⁻⁵ M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 μM non essential amino acids (Gibco), 1 mM sodium pyruvate (Gibco), mercaptoethanol 5.5×10⁻⁵ M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LP S) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and positive selection. Then CD45RO beads were used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and plated at 10⁶ cells/ml onto Falcon 6 well tissue culture plates that had been coated overnight with 0.5 μg/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶ cells/ml in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24,48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶ cells/ml in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and IL-2 (4 ng/ml). IL-12 (5 ng/ml) and anti-IL4 (1 μg/ml) were used to direct to Th1, while IL-4 (5 ng/ml) and anti-IFN gamma (1 μg/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 μg/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵ cells/ml for 8 days, changing the media every 3 days and adjusting the cell concentration to 5×10⁵ cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 10⁷ cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20 degrees C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7 μl RNAsin and 8 μl DNAse were added. The tube was incubated at 37 degrees C. for 30 minutes to remove contaminating genomic DNA, extracted once with phenol chloroform and re-precipitated with 1/10 volume of 3 M sodium acetate and 2 volumes of 100% ethanol. The RNA was spun down and placed in RNAse free water. RNA was stored at −80 degrees C.

### Panels CNSD.01, CNS_{—}1 and CNS_{—}1.1

The plates for Panel CNSD.01, CNS_{—}1 and CNS1.1 include two control wells and 94 test samples comprised of cDNA isolated from postmortem human brain tissue obtained from the Harvard Brain Tissue Resource Center. Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains two brains from each of the following diagnoses: Alzheimer's disease, Parkinson's disease, Huntington's disease, Progressive Supernuclear Palsy, Depression, and “Normal controls”. Within each of these brains, the following regions are represented: cingulate gyrus, temporal pole, globus palladus, substantia nigra, Brodman Area 4 (primary motor strip), Brodman Area 7 (parietal cortex), Brodman Area 9 (prefrontal cortex), and Brodman area 17 (occipital cortex). Not all brain regions are represented in all cases; e.g., Huntington's disease is characterized in part by neurodegeneration in the globus palladus, thus this region is impossible to obtain from confirmed Huntington's cases. Likewise Parkinson's disease is characterized by degeneration of the substantia nigra making this region more difficult to obtain. Normal control brains were examined for neuropathology and found to be free of any pathology consistent with neurodegeneration.

In the labels employed to identify tissues in the CNS panel, the following abbreviations are used:

PSP=Progressive supranuclear palsy

Sub Nigra=Substantia nigra

Glob Palladus=Globus palladus

Temp Pole=Temporal pole

Cing Gyr=Cingulate gyrus

BA 4=Brodman Area 4

### Panel CNS_Neurodegeneration_V1.0

The plates for Panel CNS_Neurodegeneration_V1.0 include two control wells and 47 test samples comprised of cDNA isolated from postmortem human brain tissue obtained from the Harvard Brain Tissue Resource Center (McLean Hospital) and the Human Brain and Spinal Fluid Resource Center (VA Greater Los Angeles Healthcare System). Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains six brains from Alzheimer's disease (AD) pateins, and eight brains from “Normal controls” who showed no evidence of dementia prior to death. The eight normal control brains are divided into two categories: Controls with no dementia and no Alzheimer's like pathology (Controls) and controls with no dementia but evidence of severe Alzheimer's like pathology, (specifically senile plaque load rated as level 3 on a scale of 0-3; 0=no evidence of plaques, 3=severe AD senile plaque load). Within each of these brains, the following regions are represented: hippocampus, temporal cortex (Brodman Area 21), parietal cortex (Brodman area 7), and occipital cortex (Brodman area 17). These regions were chosen to encompass all levels of neurodegeneration in AD. The hippocampus is a region of early and severe neuronal loss in AD; the temporal cortex is known to show neurodegeneration in AD after the hippocampus; the parietal cortex shows moderate neuronal death in the late stages of the disease; the occipital cortex is spared in AD and therefore acts as a “control” region within AD patients. Not all brain regions are represented in all cases.

In the labels employed to identify tissues in the CNS_Neurodegeneration_V1.0 panel, the following abbreviations are used:

AD=Alzheimer's disease brain; patient was demented and showed AD-like pathology upon autopsy

Control=Control brains; patient not demented, showing no neuropathology

Control (Path)=Control brains; patient not demented but showing severe AD-like pathology

Sup Temporal Ctx=Superior Temporal Cortex

if Temporal Ctx=Inferior Temporal Cortex

A. Clone Identification No: AC009485_A (PTMA 1)

Expression of gene AC009485_A was assessed using the primer-probe set Ag 184, described in Table 4. Results from RTQ-PCR runs are shown in Tables 5, 6, 7, and 8.

TABLE 4
Probe Name Ag184

| | | | Start | SEQ ID |
|---|---|---|---|---|
| Primers | Sequences | Length | Position | NO: |
| Forward | 5′-AGAGGAAGCTGAGTCTGCTACAGG-3′ | 24 | 234 | 27 |
| Probe | TET-5′- | 25 | 259 | 28 |
| | CCTCATCATCTTCAGCTGCCCGCTT-3′- | | | |
| | TAMRA | | | |
| Reverse | 5′-TCTGCTTCTTGGTATCGACATCAT-3′ | 24 | 287 | 29 |

TABLE 5
Panel 1.3D

| | Relative |
|---|---|
| | Expression(%) |
| | 1.3dtm4665t_ |
| Tissue | ag184 |
| Liver adenocarcinoma | 54.7 |
| Pancreas | 2.4 |
| Pancreatic Ca. CAPAN 2 | 17.4 |
| Adrenal gland | 4.9 |
| Thyroid | 19.5 |
| Salivary gland | 4.0 |
| Pituitary gland | 3.1 |
| Brain (fetal) | 0.0 |
| Brain (whole) | 7.7 |
| Brain (amygdala) | 6.2 |
| Brain (cerebellum) | 9.2 |
| Brain (hippocampus) | 10.7 |
| Brain (substantia nigra) | 5.5 |
| Brain (thalamus) | 7.7 |
| Cerebral Cortex | 39.8 |
| Spinal cord | 25.7 |
| CNS ca. (glio/astro) U87-MG | 27.4 |
| CNS ca. (gilo/astro) U-118-MG | 15.8 |
| CNS ca. (astro) SW1783 | 28.7 |
| CNS ca.* (neuro; met) SK-N- | 30.1 |
| AS | |
| CNS ca. (astro) SF-539 | 21.3 |
| CNS ca. (astro) SNB-75 | 11.6 |
| CNS Ca. (glio) SNB-19 | 19.6 |
| CNS Ca. (glio) U251 | 10.4 |
| CNS Ca. (glio) SF-295 | 12.3 |
| Heart (fetal) | 13.3 |
| Heart | 6.2 |
| Fetal Skeletal | 39.2 |
| Skeletal muscle | 7.4 |
| Bone marrow | 10.0 |
| Thymus | 100.0 |
| Spleen | 17.7 |
| Lymph node | 8.9 |
| Colorectal | 45.1 |
| Stomach | 12.3 |
| Small intestine | 14.3 |
| Colon Ca. SW480 | 24.8 |
| Colon ca.* (SW480 | 33.0 |
| met) SW620 | |
| Colon Ca. HT29 | 52.5 |
| Colon Ca. HCT-116 | 42.3 |
| Colon Ca. CaCo-2 | 23.7 |
| 83219 CC Well to Mod Diff | 40.6 |
| (ODO3866 | |
| Colon Ca. HCC-2998 | 19.5 |
| Gastric ca.* (liver met) NCI- | 32.1 |
| N87 | |
| Bladder | 53.2 |
| Trachea | 20.7 |
| Kidney | 14.9 |
| Kidney (fetal) | 26.6 |
| Renal ca. 786-0 | 10.8 |
| Renal Ca. A498 | 8.9 |
| Renal ca. RXF 393 | 10.7 |
| Renal ca. ACHN | 15.1 |
| Renal Ca. UO-31 | 19.3 |
| Renal Ca. TK-10 | 10.5 |
| Liver | 2.6 |
| Liver (fetal) | 9.2 |
| Liver ca. (hepatoblast) HepG2 | 8.8 |
| Lung | 16.0 |
| Lung (fetal) | 17.3 |
| Lung Ca. (small cell) LX-1 | 24.5 |
| Lung Ca. (small cell) NCI-H69 | 10.4 |
| Lung Ca. (s.cell var.) SHP-77 | 46.0 |
| Lung ca. (large cell) NCI-H460 | 8.4 |
| Lung ca. (non-sm. cell) A549 | 8.4 |
| Lung ca. (non-s.cell) NCI-H23 | 29.7 |
| Lung ca (non-s.cell) HOP-62 | 11.0 |
| Lung ca. (non-s.d) NCI-H522 | 8.0 |
| Lung ca. (squam.) SW 900 | 13.5 |
| Lung ca. (squam.) NCI-H596 | 17.4 |
| Mammary gland | 15.6 |
| Breast ca.* (pl. effusion) MCF-7 | 37.6 |
| Breast ca.* (pl.ef) MDA-MB- | 12.6 |
| 231 | |
| Breast ca.* (pl. effusion) T47D | 8.0 |
| Breast ca. BT-549 | 12.6 |
| Breast ca. MDA-N | 22.8 |
| Ovary | 61.6 |
| Ovarian Ca. OVCAR-3 | 19.6 |
| Ovarian Ca. OVCAR-4 | 4.2 |
| Ovarian Ca. OVCAR-5 | 21.2 |
| Ovarian Ca. OVCAR-8 | 26.6 |
| Ovarian Ca. IGROV-1 | 6.2 |
| Ovarian ca.* (ascites) SK-OV-3 | 22.4 |
| Uterus | 8.2 |
| Placenta | 10.7 |
| Prostate | 8.4 |
| Prostate ca.* (bone met) PC-3 | 8.8 |
| Testis | 5.0 |
| Melanoma Hs688 (A).T | 7.6 |
| Melanoma* (met) Hs688 (B).T | 6.2 |
| Melanoma UACC-62 | 3.9 |
| Melanoma M14 | 5.1 |
| Melanoma LOX IMVI | 10.0 |
| Melanoma* (met) SK-MEL-5 | 6.9 |
| Adipose | 14.9 |

TABLE 6
Panel 2D

| | Relative |
|---|---|
| | Expression(%) |
| | 2dx4tm4640t_ |
| Tissue Name | ag184_b2 |
| Normal Colon GENPAK | 55.6 |
| 061003 | |
| 83219 CC Well to Mod Diff | 21.7 |
| (ODO3866) | |
| 83220 CC NAT (ODO3866) | 15.3 |
| 83221 CC Gr.2 rectosigmoid | 9.8 |
| (ODO3868) | |
| 83222 CC NAT (ODO3868) | 5.6 |
| 83235 CC Mod Diff | 48.3 |
| (ODO3820) | |
| 83236 CC NAT (ODO3920) | 14.5 |
| 83237 CC Gr.2 ascend colon | 51.9 |
| (ODO3921) | |
| 83238 CC NAT (ODO3921) | 11.4 |
| 83241 CC from Partial | 48.8 |
| Hepatectomy (ODO4309) | |
| 83242 Liver NAT (ODO4309) | 9.5 |
| 87472 Colon mets to lung | 21.2 |
| (OD04451-01) | |
| 87473 Lung NAT (OD04451- | 15.2 |
| 02) | |
| Normal Prostate Clontech A + | 62.5 |
| 6546-1 | |
| 84140 Prostate Cancer | 29.3 |
| (OD04410) | |
| 84141 Prostate NAT | 22.3 |
| (OD04410) | |
| 87073 Prostate Cancer | 22.1 |
| (OD04720-01) | |
| 87074 Prostate NAT | 32.2 |
| (OD04720-02) | |
| Normal Lung GENPAK 061010 | 35.1 |
| 83239 Lung Met to Muscle | 33.9 |
| (ODO4286) | |
| 83240 Muscle NAT | 7.6 |
| (ODO4286) | |
| 84136 Lung Malignant Cancer | 52.0 |
| (OD03126) | |
| 84137 Lung NAT (OD03126) | 33.1 |
| 84871 Lung Cancer (OD04404) | 62.9 |
| 84872 Lung NAT (OD04404) | 21.0 |
| 84875 Lung Cancer (OD04565) | 17.0 |
| 84876 Lung NAT (OD04565) | 12.3 |
| 85950 Lung Cancer (OD04237- | 58.3 |
| 01) | |
| 85970 Lung NAT (OD04237- | 29.1 |
| 02) | |
| 83255 Ocular Mel Met to Liver | 20.1 |
| (ODO4310) | |
| 83256 Liver NAT (ODO4310) | 8.5 |
| 84139 Melanoma Mets to Lung | 33.9 |
| (OD04321) | |
| 84138 Lung NAT (OD04321) | 26.7 |
| Normal Kidney GENPAK | 30.3 |
| 061008 | |
| 83786 Kidney Ca, Nuclear | 34.2 |
| grade 2 (OD04338) | |
| 83787 Kidney NAT (OD04338) | 2.0 |
| 83788 Kidney Ca Nuclear grade | 31.2 |
| 1/2 (OD04339) | |
| 83789 Kidney NAT (OD04339) | 23.7 |
| 83790 Kidney Ca, Clear cell | 33.5 |
| type (OD04340) | |
| 83791 Kidney NAT (OD04340) | 22.4 |
| 83792 Kidney Ca, Nuclear | 13.3 |
| grade 3 (OD04348) | |
| 83793 Kidney NAT (OD04348) | 23.2 |
| 87474 Kidney Cancer | 12.7 |
| (OD04622-01) | |
| 87475 Kidney NAT (OD04622- | 5.6 |
| 03) | |
| 85973 Kidney Cancer | 28.6 |
| (OD04450-01) | |
| 85974 Kidney NAT (OD04450- | 24.8 |
| 03) | |
| Kidney Cancer Clontech | 12.4 |
| 8120607 | |
| Kidney NAT Clontech 8120608 | 7.1 |
| Kidney Cancer Clontech | 8.7 |
| 8120613 | |
| Kidney NAT Clontech 8120614 | 7.3 |
| Kidney Cancer Clontech | 17.2 |
| 9010320 | |
| Kidney NAT Clontech 9010321 | 10.9 |
| Normal Uterus GENPAK | 8.4 |
| 061018 | |
| Uterus Cancer GENPAK | 36.6 |
| 064011 | |
| Normal Thyroid Clontech A + | 34.9 |
| 6570-1 | |
| Thyroid Cancer GENPAK | 30.9 |
| 064010 | |
| Thyroid Cancer INVITROGEN | 17.0 |
| A302152 | |
| Thyroid NAT INVITROGEN | 32.2 |
| A302153 | |
| Normal Breast GENPAK | 28.0 |
| 061019 | |
| 84877 Breast Cancer | 39.6 |
| (OD04566) | |
| 85975 Breast Cancer | 51.9 |
| (OD04590-01) | |
| 85976 Breast Cancer Mets | 56.7 |
| (OD04590-03) | |
| 87070 Breast Cancer Metastasis | 41.2 |
| (OD04655-05) | |
| GENPAK Breast Cancer | 23.7 |
| 064006 | |
| Breast Cancer Res. Gen. 1024 | 34.6 |
| Breast Cancer Clontech | 46.5 |
| 9100266 | |
| Breast NAT Clontech 9100265 | 20.9 |
| Breast Cancer INVITROGEN | 57.0 |
| A209073 | |
| Breast NAT INVITROGEN | 23.5 |
| A2090734 | |
| Normal Liver GENPAK | 7.0 |
| 061009 | |
| Liver Cancer GENPAK 064003 | 8.8 |
| Liver Cancer Research | 4.6 |
| Genetics RNA 1025 | |
| Liver Cancer Research | 10.3 |
| Genetics RNA 1026 | |
| Paired Liver Cancer Tissue | 7.0 |
| Research Genetics RNA 6004-T | |
| Paired Liver Tissue Research | 10.6 |
| Genetics RNS 6004-N | |
| Paired Liver Cancer Tissue | 10.2 |
| Research Genetics RNA 6005-T | |
| Paired Liver Tissue Research | 2.0 |
| Genetics RNA 6005-N | |
| Normal Bladder GENPAK | 48.2 |
| 061001 | |
| Bladder Cancer Research | 19.4 |
| Genetics RNA 1023 | |
| Bladder Cancer INVITROGEN | 29.3 |
| A302173 | |
| 87071 Bladder Cancer | 57.6 |
| (OD04718-01) | |
| 87072 Bladder Normal | 26.7 |
| Adjacent (OD04718-03) | |
| Normal Ovary Res. Gen. | 16.4 |
| Ovarian Cancer GENPAK | 53.7 |
| 064008 | |
| 87492 Ovary Cancer | 100.0 |
| (OD04768-07) | |
| 87493 Ovary NAT (OD04768- | 8.0 |
| 08) | |
| Normal Stomach GENPAK | 27.3 |
| 061017 | |
| Gastric Cancer Clontech | 12.6 |
| 9060358 | |
| NAT Stomach Clontech | 17.8 |
| 9060359 | |
| Gastric Cancer Clontech | 35.4 |
| 9060395 | |
| NAT Stomach Clontech | 23.6 |
| 9060394 | |
| Gastric Cancer Clontech | 51.6 |
| 9060397 | |
| NAT Stomach Clontech | 10.4 |
| 90600396 | |
| Gastric Cancer GENPAK | 62.5 |
| 064005 | |

TABLE 7
Panel 4D

| | Relative |
|---|---|
| | Expression(%) |
| | 4dx45tm4605t_ |
| Tissue Name | ag184_a2 |
| 93768_Secondary Th1_anti- | 14.0 |
| CD28/anti-CD3 | |
| 93769_Secondary Th2_anti- | 16.2 |
| CD28/anti-CD3 | |
| 93770_Secondary Tr1_anti- | 12.3 |
| CD28/anti-CD3 | |
| 93573_Secondary Th1_resting | 1.4 |
| day 4-6 in IL-2 | |
| 93572_Secondary Th2_resting | 3.3 |
| day 4-6 in IL-2 | |
| 93571_Secondary Tr1_resting | 3.2 |
| day 4-6 in IL-2 | |
| 93568_primary Th1_anti- | 17.3 |
| CD28/anti-CD3 | |
| 93569_primary Th2_anti- | 16.1 |
| CD28/anti-CD3 | |
| 93570_primary Tr1_anti- | 21.2 |
| CD28/anti-CD3 | |
| 93565_primary Th1_resting dy | 25.4 |
| 4-6 in IL-2 | |
| 93566_primary Th2_resting dy | 10.6 |
| 4-6 in IL-2 | |
| 93567_primary Tr1_resting dy | 10.0 |
| 4-6 in IL-2 | |
| 93351_CD45RA CD4 | 5.8 |
| lymphocyte_anti-CD28/anti- | |
| CD3 | |
| 93352_CD45RO CD4 | 14.7 |
| lymphocyte_anti-CD28/anti- | |
| CD3 | |
| 93251_CD8 Lymphocytes_ | 14.8 |
| anti-CD28/anti-CD3 | |
| 93353_chronic CD8 | 14.0 |
| Lymphocytes 2ry_resting dy 4- | |
| 6 in IL-2 | |
| 93574-chronic CD8 | 9.6 |
| Lymphocytes 2ry_activated | |
| CD3/CD28 | |
| 93354_CD4_none | 2.2 |
| 93252_Secondary | 5.3 |
| Th1/Th2/Tr1_anti-CD95 CH11 | |
| 93103_LAK cells_resting | 7.0 |
| 93788_LAK cells_IL-2 | 12.6 |
| 93787_LAK cells_IL-2 + | 10.6 |
| IL-12 | |
| 93789_LAK cells_IL-2 + IFN | 17.5 |
| gamma | |
| 93790_LAK cells_IL-2 + | 11.9 |
| IL-18 93104_LAK | 6.6 |
| cells_PMA/ionomycin and | |
| IL-18 | |
| 93578_NK Cells IL-2_resting | 6.2 |
| 93109_Mixed Lymphocyte | 7.0 |
| Reaction_Two Way MLR | |
| 93110_Mixed Lymphocyte | 8.7 |
| Reaction_Two Way MLR | |
| 93111_Mixed Lymphocyte | 6.4 |
| Reaction_Two Way MLR | |
| 93112_Mononuclear Cells | 4.5 |
| (PBMCs)_resting | |
| 93113_Mononuclear Cells | 38.4 |
| (PBMCs)_PWN | |
| 93114_Mononuclear Cells | 15.5 |
| (PBMCs)_PHA-L | |
| 93249_Ramos (B cell)_none | 26.7 |
| 93250_Ramos (B | 100.0 |
| cell)_ionomycin | |
| 93349_B lymphocytes_PWM | 63.8 |
| 93350_B lymphoytes_CD40L | 15.8 |
| and IL-4 | |
| 92665_EOL-1 | 15.8 |
| (Eosinophil)_dbcAMP | |
| differentiated | |
| 93248_EOL-1 | 7.1 |
| (Eosinophil)_dbcAMP/ | |
| PMAionomycin | |
| 93356_Dendritic Cells_none | 4.9 |
| 93355_Dendritic Cells_LPS | 4.1 |
| 100 ng/ml | |
| 93775_Dendritic Cells_anti- | 6.6 |
| CD40 | |
| 93774_MOnocytes_resting | 3.8 |
| 93776_Monocytes_LPS 50 | 1.6 |
| ng/ml | |
| 93581_Macrophages_resting | 7.2 |
| 93582_Macrophages_LPS 100 | 1.8 |
| ng/ml | |
| 93098_HUVEC | 10.7 |
| (Endothelial)_none | |
| 93099_HUVEC | 17.3 |
| (Endothelial)_starved | |
| 93100_HUVEC | 6.6 |
| (Endothelial)_IL-1b | |
| 93779_HUVEC | 10.3 |
| (Endothelial)_IFN gamma | |
| 93102_HUVEC | 8.9 |
| (Endothelial)_TNF alpha + IFN | |
| gamma | |
| 93101_HUVEC | 8.9 |
| (Endothelial)_TNF alpha + IL4 | |
| 93781_HUVEC | 6.7 |
| (Endothelial)_IL-11 | |
| 93583_Lung Microvascular | 6.6 |
| Endothelial Cells_none | |
| 93584_Lung Microvascular | 7.4 |
| Endothelial Cells_TNFa (4 | |
| ng/ml) and IL 1b (1 ng/ml) | |
| 92662_Microvascular Dermal | 10.8 |
| endothelium_none | |
| 92663_MIcrosvasular Dermal | 6.8 |
| endothelium_TNFa (4 ng/ml) | |
| and IL 1b (1 ng/ml) | |
| 93773_Bronchial | 5.2 |
| epithelium_TNFa (4 ng/ml) and | |
| IL 1b (1 ng/ml)** | |
| 93347_Small Airway | 2.6 |
| Epithelium_none | |
| 93348_Small Airway | 12.6 |
| Epithelium_TNFa (4 ng/ml) | |
| and IL 1b (1 ng/ml) | |
| 92668_Coronery Artery | 5.8 |
| SMC_resting | |
| 92669_Coronery Artery | 1.9 |
| SMC_TNFa (4 ng/ml) and IL | |
| 1b (1 ng/ml) | |
| 93107_astrocytes_resting | 1.6 |
| 93108_astrocytes_TNFa (4 | 1.1 |
| ng/ml) and IL 1b (1 ng/ml) | |
| 92666_KU-812 | 14.9 |
| (Basophil)_resting | |
| 92667_KU-812 | 31.2 |
| (Basophil)_PMA/ionoycin | |
| 93579_CCD1106 | 7.1 |
| (Keratinocytes)_none | |
| 93580_CCD1106 | 3.5 |
| (Keratinocytes)_TNFa and | |
| IFNg** | |
| 93791_Liver Cirrhosis | 1.1 |
| 93792_Lupus Kidney | 0.9 |
| 93577_NCI-H292 | 12.3 |
| 93358_NCI-H292_IL-4 | 15.6 |
| 93360_NCI-H292_IL-9 | 16.9 |
| 93359_NCI-H292_IL-13 | 10.7 |
| 93357_NCI-H292_IFN gamma | 9.3 |
| 93777_HPAEC_- | 6.7 |
| 93778_HPAEC_IL-1 beta/TNA | 5.6 |
| alpha | |
| 93254_Normal Human Lung | 3.8 |
| Fibroblast_none | |
| 93253_Normal Human Lung | 4.0 |
| Fibroblast_TNFa (4 ng/ml) and | |
| IL-1b (1 ng/ml) | |
| 93257_Normal Human Lung | 8.4 |
| Fibroblast_IL-4 | |
| 93256_Normal Human Lung | 6.8 |
| Fibroblast_IL-9 | |
| 93255_Normal Human Lung | 6.2 |
| Fibroblast_IL-13 | |
| 93258_Normal Human Lung | 9.7 |
| Fibroblast_IFN gamma | |
| 93106_Dermal Fibroblasts | 10.1 |
| CCD1070_resting | |
| 93361_Dermal Fibroblasts | 31.6 |
| CCD1070_TNF alpha 4 ng/ml | |
| 93105_Dermal Fibroblasts | 4.9 |
| CCD1070_IL-1 beta 1 ng/ml | |
| 93772_dermal fibroblast_IFN | 4.2 |
| gamma | |
| 93771_dermal fibroblast_IL-4 | 7.1 |
| 93260_IBD Colitis 2 | 1.4 |
| 93261_IBD Crohns | 0.8 |
| 735010_Colon_normal | 6.6 |
| 735019_Lung_none | 6.9 |
| 64028-1_Thymus_none | |
| 64030-1_Kidney_none | 19.6 |

TABLE 8
CNS_neurodegeneration_panel_v1.0

| | Relative |
|---|---|
| | Expression (%) |
| | tm6945t_ |
| Tissue Name | ag184_b1 |
| AD 1 Hippo | 23.8 |
| AD 2 Hippo | 31.4 |
| AD 3 Hippo | 17.9 |
| AD 4 Hippo | 18.7 |
| AD 5 hippo | 75.3 |
| AD 6 Hippo | 83.3 |
| Control 2 Hippo | 41.8 |
| Control 4 Hippo | 16.6 |
| Control (Path) 3 Hippo | 11.9 |
| AD 1 Temporal Ctx | 42.9 |
| AD 2 Temporal Ctx | 39.6 |
| AD 3 Temporal Ctx | 14.8 |
| AD 4 Temporal Ctx | 37.7 |
| AD 5 Inf Temporal Ctx | 100.0 |
| AD 5 Sup Temporal Ctx | 51.7 |
| AD 6 Inf Temporal Ctx | 88.9 |
| AD 6 Sup Temporal Ctx | 76.2 |
| Control 1 Temporal Ctx | 12.4 |
| Control 2 Temporal Ctx | 33.1 |
| Control 3 Temporal Ctx | 21.4 |
| Control 4 Temporal Ctx | 12.7 |
| Control (Path) 1 Temporal Ctx | 48.1 |
| Control (Path) 2 Temporal Ctx | 34.8 |
| Control (Path) 3 Temporal Ctx | 9.3 |
| Control (Path) 4 Temporal Ctx | 34.7 |
| AD 1 Occipital Ctx | 32.8 |
| AD 2 Occipital Ctx (Missing) | 0.0 |
| AD 3 Occipital Ctx | 13.3 |
| AD 4 Occipital Ctx | 36.4 |
| AD 5 Occipital Ctx | 44.4 |
| AD 6 Occipital Ctx | 58.6 |
| Control 1 Occipital Ctx | 11.2 |
| Control 2 Occipital Ctx | 44.5 |
| Control 3 Occipital Ctx | 22.3 |
| Control 4 Occipital Ctx | 16.3 |
| Control (Path) 1 Occipital Ctx | 78.3 |
| Control (Path) 2 Occipital Ctx | 17.5 |
| Control (Path) 3 Occipital Ctx | 8.8 |
| Control (Path) 4 Occipital Ctx | 22.9 |
| Control 1 Parietal Ctx | 15.6 |
| Control 2 Parietal Ctx | 57.3 |
| Control 3 Parietal Ctx | 17.2 |
| Control (Path) 12 Parietal Ctx | 49.1 |
| Control (Path) 2 Parietal Ctx | 28.6 |
| Control (Path) 3 Parietal Ctx | 10.1 |
| Control (Path) 4 Parietal Ctx | 40.3 |

Panel 1.3D Summary: Ag184 The AC009485_A gene encodes a protein with strong homology to prothymosin alpha. Expression of the AC009485_A gene is highest in thymus (CT=20.8). Overall, there is lower but widespread expression of this gene in all tissues and cell lines on this panel except fetal brain. Thus, the expression of the AC009485_A gene could be used to distinguish thymus from other tissues and the absence of expression could be used to distinguish fetal brain tissue from other tissues.

Among CNS samples, AC009485_A gene expression is highest in cerebral cortex and spinal cord (CT=22-23) with lower expression in thalamus, substantia nigra, hippocampus, cerebellum and amygdala. Please see CNS_neurodegeneration_panel_v10 for a description of the potential utility of this gene in treating CNS disorders.

The AC009485_A gene is also expressed at high levels in metabolic tissues including adipose, pancreas, adrenal gland, pituitary gland, thyroid, adult/fetal heart, and adult/fetal skeletal muscle (CTs=22-26). Therefore, this gene product may be involved in the pathogenesis and/or treatment of disease in any or all of these tissues.

Panel 2D Summary: Ag184 Expression of the AC009485_A gene is highest in an ovarian cancer sample (CT=20.5) on this panel. Overall there appears to be a consistent association with the higher expression of this gene in cancerous tissues. The AC009485_A gene is overexpressed in cancerous tissue from stomach, ovary, lung and colon when compared to normal adjacent tissue. Thus, the expression of this gene could be used as a marker to distinguish between stomach, ovarian, lung or colon cancers and their respective normal adjacent tissues and could be of benefit for the diagnosis or prognostication of these diseases. Moreover, therapeutic modulation of the expression or activity of the AC009485_A gene, through the use of small molecule drugs, antibodies or protein therapeutics might be beneficial for the treatment of stomach, ovarian, lung or colon cancer.

Panel 4D Summary: Ag184 Expression of the AC009485_A gene is highest in ionomycin-treated Ramos B cells (CT=18.4). Consistent with these data, this gene is also found highly expressed on activated primary B cells (B cells+PWM and PBMC+PWM). In addition, the AC009485_A gene is expressed at lower levels in TNF-alpha stimulated dermal fibroblasts and activated basophils. This gene encodes a protein that has homology to thymosin alpha-1, a peptide originally isolated from thymus, which is consistent with the data showing strong expression in the thymus in Panel 1.3D. Thymosin alpha has been demonstrated to have potent immunostimulatory activities. Therefore the use of the AC009485_A gene product as a protein therapeutic could be useful for improving immune disorders associated with various diseases such as AIDS or other immune deficiency disorders as well as for an adjuvant for cancer chemotherapy or viral mediated diseases. The observation that the AC009485_A gene is expressed in activated B cells suggest that antibody against protein encoded by this gene might be useful to prevent B cell expansion or hyper proliferation as seen in B cell lymphomas.

CNS_neurodegeneration_panel_v1.0 Summary: Ag 184 Expression of the AC009485_A gene is highest in inferior and superior temporal cortex of two Alzheimer's disease patients (CT=23). Thus, this gene is slightly (but detectably) increased in the temporal cortex of Alzheimer's disease patients (1.5-fold increase); this increase is detectable after correcting CT values for overall mRNA quality. Thymosin alpha is critical for maintenance of levels of both nerve growth factor (NGF) and its receptor (p75NGFr); early thymectomy decreases levels of both of these proteins, which are partially restored by injection of thymosin alpha. NGF in turn is critical for the formation and maintenance of basal cholinergic neurons, a population of neurons that specifically degenerate in Alzheimer's disease (AD). The increase in levels of this protein seen in AD may be an attempt on the part of the CNS to compensate for the loss of cholinergic neurons. Therefore, therapeutic modulation of the AC009485_A gene or its protein product in Alzheimer's disease may be useful in slowing neurodegeneration of cholinergic neurons, especially when used in conjunction with acetylcholine esterase inhibitors (a standard of AD therapy). (Turrini et al., A role of the thymus and thymosin-alpha1 in brain NGF levels and NGF receptor expression. J. Neuroimmunol. 82:64-72, 1998; Turrini and Aloe, Evidence that endogenous thymosin alpha-1 is present in the rat central nervous system. Neurochem. int. 35:463-70. 1999; Fahnestock et al., The precursor pro-nerve growth factor is the predominant form of nerve growth factor in brain and is increased in alzheimer's disease. Mol. Cell. Neurosci. 18:210-220.2001.)

B. Clone Identification No: AC010175_A.0.1/CG53252-01 (PTMA 2)

Expression of gene AC010175_A.0.1 was assessed using the primer-probe sets Ag165, Ag185b, and Ag3750, described in Tables 9 and 10. Results from RTQ-PCR runs are shown in Tables 11, 12, 13, 14, 15, 16, and 17.

TABLE 9
Probe Name Ag165/Ag185b (identical sequences)

| | | | | Start | SEQ ID |
|---|---|---|---|---|---|
| Primers | Sequences | TM | Length | Position | NO: |
| Forward | 5′-ATGTCAGACGCAGCCGTAGA-3′ | | 20 | 1 | 30 |
| Probe | TET-5′- | | 24 | 22 | 31 |
| | ACCAGCTCCGAAATCACCACCGAG-3′- | | | | |
| | TAMRA | | | | |
| Reverse | 5′-CTTCCACAACTTCCTTCTTCTCCT-3′ | | 24 | 53 | 32 |

TABLE 10
Probe Name Ag3750

| | | | | Start | SEQ ID |
|---|---|---|---|---|---|
| Primers | Sequences | TM | Length | Position | NO: |
| Forward | 5′-GTCAGACGCAGGCGTAGAC-3′ | 59.6 | 19 | 3 | 33 |
| Probe | FAM-5′- | 69 | 26 | 27 | 34 |
| | CTCCGAAATCACCACCGAGGAGTTAA- | | | | |
| | 3′-TAMRA | | | | |
| Reverse | 5′-CCTCTTCCACAACTTCCTTCTT-3′ | 58.9 | 22 | 58 | 35 |

TABLE 11
Panel 1

| | Relative |
|---|---|
| | Expression (%) |
| Tissue Name | tm442f |
| Endothelial cells | 9.2 |
| Endothelial cells (treated) | 34.2 |
| Pancreas | 0.0 |
| Pancreatic ca. CAPAN 2 | 31.0 |
| Adrenal gland | 44.8 |
| Thyroid | 44.4 |
| Salivary gland | 22.4 |
| Pituitary gland | 8.8 |
| Brain (fetal) | 18.4 |
| Brain (whole) | 34.2 |
| Brain (amygdala) | 22.2 |
| Brain (cerebellum) | 54.7 |
| Brain (hippocampus) | 19.2 |
| Brain (substantia nigra) | 35.6 |
| Brain (thalamus) | 27.7 |
| Brain (hypothalamus) | 8.3 |
| Spinal cord | 26.2 |
| CNS ca. (glio/astro) U87-MG | 10.7 |
| CNS ca. (glio/astro) U-118-MG | 14.1 |
| CNS ca. (astro) SW1783 | 9.1 |
| CNS ca.* (neuro; met) SK-N- | 33.0 |
| AS | |
| CNS ca. (astro) SF-539 | 8.2 |
| CNS ca. (astro) SNB-75 | 9.5 |
| CNS ca. (glio) SNB-19 | 14.1 |
| CNS ca. (glio) U251 | 7.4 |
| CNS ca. (glio) SF-295 | 7.2 |
| Heart | 10.1 |
| Skeletal muscle | 17.0 |
| Bone marrow | 43.8 |
| Thymus | 73.7 |
| Spleen | 30.6 |
| Lymph node | 31.2 |
| Colon (ascending) | 29.9 |
| Stomach | 36.9 |
| Small intestine | 11.8 |
| Colon ca. SW480 | 7.2 |
| Colon ca.* (SW480 | 17.2 |
| met) SW620 | |
| Colon ca. HT29 | 24.5 |
| Colon ca. HCT-116 | 82.9 |
| Colon ca. CaCo-2 | 18.2 |
| Colon ca. HCT-15 | 21.9 |
| Colon ca. HCC-2998 | 20.0 |
| Gastric ca.* (liver met) NCI- | 37.1 |
| N87 | |
| Bladder | 25.7 |
| Trachea | 39.5 |
| Kidney | 14.2 |
| Kidney (fetal) | 33.4 |
| Renal ca. 786-0 | 16.5 |
| Renal ca. A498 | 2.3 |
| Renal ca. RXF 393 | 6.9 |
| Renal ca. ACHN | 12.4 |
| Renal ca. UO-31 | 6.0 |
| Renal ca. TK-10 | 11.3 |
| Liver | 23.3 |
| Liver (fetal) | 14.1 |
| Liver ca. (hepatoblast) HepG2 | 7.6 |
| Lung | 13.6 |
| Lung (fetal) | 27.2 |
| Lung ca. (small cell) LX-1 | 28.5 |
| Lung ca. (small cell) NCI-H69 | 15.2 |
| Lung ca. (s.cell var.) SHP-77 | 98.6 |
| Lung ca. (large cell) NCI-H460 | 27.4 |
| Lung ca. (non-sm. cell) A549 | 12.9 |
| Lung ca. (non-s.cell) NCI-H23 | 19.3 |
| Lung ca (non-s.cell) HOP-62 | 8.3 |
| Lung ca. (non-s.cl) NCI-H522 | 35.6 |
| Lung ca. (squam.) SW 900 | 43.5 |
| Lung ca. (squam.) NCI-H596 | 34.4 |
| Mammary gland | 37.6 |
| Breast ca.* (pl. effusion) MCF-7 | 57.0 |
| Breast ca.* (pl.ef) MDA-MB- | 12.4 |
| 231 | |
| Breast ca.* (pl. effusion) T47D | 28.9 |
| Breast ca. BT-549 | 42.9 |
| Breast ca. MDA-N | 36.6 |
| Ovary | 26.4 |
| Ovarian ca. OVCAR-3 | 15.2 |
| Ovarian ca. OVCAR-4 | 10.9 |
| Ovarian ca. OVCAR-5 | 25.5 |
| Ovarian ca. OVCAR-8 | 19.9 |
| Ovarian ca. IGROV-1 | 7.6 |
| Ovarian ca.* (ascites) SK-OV-3 | 23.0 |
| Uterus | 18.6 |
| Placenta | 15.5 |
| Prostate | 51.4 |
| Prostate ca.* (bone met) PC-3 | 33.2 |
| Testis | 29.3 |
| Melanoma Hs688 (A).T | 13.1 |
| Melanoma* (met) Hs688 (B).T | 6.0 |
| Melanoma UACC-62 | 4.1 |
| Melanoma M14 | 19.8 |
| Melanoma LOX IMVI | 100.0 |
| Melanoma* (met) SK-MEL-5 | 16.8 |
| Melanoma SK-MEL-28 | 26.1 |

Panel 1.3D

| | Relative |
|---|---|
| | Expression (%) |
| | 1.3dtm4664t_ |
| Tissue Name | ag165 |
| Liver adenocarcinoma | 98.6 |
| Pancreas | 3.3 |
| Pancreatic ca. CAPAN 2 | 25.7 |
| Adrenal gland | 7.2 |
| Thyroid | 21.3 |
| Salivary gland | 4.7 |
| Pituitary gland | 3.1 |
| Brain (fetal) | 5.6 |
| Brain (whole) | 12.4 |
| Brain (amygdala) | 7.1 |
| Brain (cerebellum) | 12.6 |
| Brain (hippocampus) | 14.8 |
| Brain (substantia nigra) | 6.8 |
| Brain (thalamus) | 11.0 |
| Cerebral Cortex | 74.7 |
| Spinal cord | 22.2 |
| CNS ca. (glio/astro) U87-MG | 43.8 |
| CNS ca. (glio/astro) U-118-MG | 25.3 |
| CNS ca. (astro) SW1783 | 40.6 |
| CNS ca.* (neuro; met ) SK-N- | 38.4 |
| AS | |
| CNS ca. (astro) SF-539 | 20.9 |
| CNS ca. (astro) SNB-75 | 14.2 |
| CNS ca. (glio) SNB-19 | 25.0 |
| CNS ca. (glio) U251 | 10.7 |
| CNS ca. (glio) SF-295 | 13.7 |
| Heart (fetal) | 23.3 |
| Heart | 11.4 |
| Fetal Skeletal | 55.9 |
| Skeletal muscle | 10.7 |
| Bone marrow | 15.2 |
| Thymus | 81.2 |
| Spleen | 16.4 |
| Lymph node | 9.2 |
| Colorectal | 84.7 |
| Stomach | 10.1 |
| Small intestine | 12.5 |
| Colon ca. SW480 | 22.1 |
| Colon ca.* (SW480 | 44.1 |
| met) SW620 | |
| Colon ca. HT29 | 80.1 |
| Colon ca. HCT-116 | 88.3 |
| Colon ca. CaCo-2 | 34.9 |
| 83219 CC Well to Mod Diff | 37.4 |
| (ODO3866) | |
| Colon ca. HCC-2998 | 24.3 |
| Gastric ca.* (liver met) NCI- | 45.7 |
| N87 | |
| Bladder | 88.9 |
| Trachea | 22.4 |
| Kidney | 18.6 |
| Kidney (fetal) | 28.5 |
| Renal ca. 786-0 | 13.6 |
| Renal ca. A498 | 9.9 |
| Renal ca. RXF 393 | 9.9 |
| Renal ca. ACHN | 15.7 |
| Renal ca. UO-31 | 25.0 |
| Renal ca. TK-10 | 12.2 |
| Liver | 3.6 |
| Liver (fetal) | 11.7 |
| Liver ca. (hepatoblast) HepG2 | 11.8 |
| Lung | 13.8 |
| Lung (fetal) | 24.0 |
| Lung ca. (small cell) LX-1 | 44.1 |
| Lung ca. (small cell) NCI-H69 | 17.2 |
| Lung ca. (s.cell var.) SHP-77 | 72.7 |
| Lung ca. (large cell)`NCI-H460 | 9.9 |
| Lung ca. (non-sm. cell) A549 | 11.6 |
| Lung ca. (non-s.cell) NCI-H23 | 33.7 |
| Lung ca (non-s.cell) HOP-62 | 15.4 |
| Lung ca. (non-s.cl) NCI-H522 | 12.2 |
| Lung ca. (squam.) SW 900 | 23.3 |
| Lung ca. (squam.) NCI-H596 | 30.1 |
| Mammary gland | 14.1 |
| Breast ca.* (pl. effusion) MCF-7 | 54.3 |
| Breast ca.* (pl.ef) MDA-MB- | 17.1 |
| 231 | |
| Breast ca.* (pl. effusion) T47D | 11.7 |
| Breast ca. BT-549 | 16.2 |
| Breast ca. MDA-N | 24.7 |
| Ovary | 100.0 |
| Ovarian ca. OVCAR-3 | 19.6 |
| Ovarian ca. OVCAR-4 | 5.6 |
| Ovarian ca. OVCAR-5 | 22.8 |
| Ovarian ca. OVCAR-8 | 31.0 |
| Ovarian ca. IGROV-1 | 5.7 |
| Ovarian ca.* (ascites) SK-OV-3 | 27.4 |
| Uterus | 7.7 |
| Placenta | 9.7 |
| Prostate | 6.6 |
| Prostate ca.* (bone met) PC-3 | 19.3 |
| Testis | 4.6 |
| Melanoma Hs688 (A).T | 7.1 |
| Melanoma* (met) Hs688 (B).T | 9.2 |
| Melanoma UACC-62 | 7.0 |
| Melanoma M14 | 12.4 |
| Melanoma LOX IMVI | 19.8 |
| Melanoma* (met) SK-MEL-5 | 17.7 |
| Adipose | 20.3 |

TABLE 13
General_screening_panel_v1.4

| | Relative |
|---|---|
| | Expression (%) |
| | tm7295f_ |
| Tissue Name | ag3750_a2 |
| D6005-01_Human adipose | 9.6 |
| 112193_Metastatic melanoma | 8.2 |
| 112192_Metastatic melanoma | 6.1 |
| 95280_Epidermis (metastatic | 43.3 |
| melanoma) | |
| 95279_Epidermis (metastatic | 46.6 |
| melanoma) | |
| Melanoma (met)_SK-MEL-5 | 17.2 |
| 112196_Tongue (oncology | 18.4 |
| 113461_Testis Pool | 5.7 |
| Prostate ca. (bone met)_PC-3 | 25.0 |
| 113455_Prostate Pool | 4.3 |
| 103396_Placenta | 5.9 |
| 113463_Uterus Pool | 7.5 |
| Ovarian carcinoma_OVCAR-3 | 30.8 |
| Ovarian | 40.9 |
| carcinoma (ascites)_SK-OV-3 | |
| 95297_Adenocarcinoma | 13.5 |
| (ovary) | |
| Ovarian carcinoma_OVCAR-5 | 34.1 |
| Ovarian carcinoma_IGROV-1 | 11.9 |
| Ovarian carcinoma_OVCAR-8 | 8.5 |
| 103368_Ovary | 7.7 |
| MCF7_breast | 24.2 |
| carcinoma (pleural effusion) | |
| Breast ca. (pleural | 23.7 |
| effusion)_MDA-MB-231 | |
| 112189_ductal cell | 100.0 |
| carcinoma(breast) | |
| Breast ca. (pleural | 70.7 |
| effusion)_T47D | |
| Breast carcinoma_MDA-N | 19.1 |
| 113452_Breast Pool | 17.7 |
| 103398_Trachea | 11.7 |
| 112354_lung | 5.2 |
| 103374_Fetal Lung | 41.7 |
| 94921_Small cell carcinoma of | 13.2 |
| the lung | |
| Lung ca. (small cell)_LX-1 | 37.8 |
| 94919_Small cell carcinoma of | 10.8 |
| the lung | |
| Lung ca.(s.cell var.)_SHP-77 | 28.4 |
| 95268_Lung (Large cell | 29.0 |
| carcinoma) | |
| 94920_Small cell carcinoma of | 18.3 |
| the lung | |
| Lung ca. (non-s.cell)_NCI-H23 | 33.4 |
| Lung ca. (large cell)_NCI-H460 | 15.8 |
| Lung ca. (non-s.cell)_HOP-62 | 9.0 |
| Lung ca. (non-s.cl)_NCI-H522 | 12.0 |
| 103392_Liver | 1.5 |
| 103393_Fetal Liver | 18.2 |
| Liver ca. (hepatoblast)_HepG2 | 6.3 |
| 113465_Kidney Pool | 13.3 |
| 103373_Fetal Kidney | 25.1 |
| Renal ca._786-0 | 19.6 |
| 112188_renal cell carcinoma | 6.8 |
| Renal ca._ACHN | 15.3 |
| 112190_Renal cell carcinoma | 19.5 |
| Renal ca._TK-10 | 19.4 |
| Bladder | 21.6 |
| Gastric ca. (liver met)_NCI-N87 | 29.4 |
| 112197_Stomach | 74.0 |
| 94938_Colon Adenocarcinoma | 21.7 |
| Colon ca._SW480 | 48.2 |
| Colon ca. (SW480 met)_SW620 | 40.8 |
| Colon ca._HT29 | 31.5 |
| Colon ca._HCT-116 | 74.0 |
| Colon ca._CaCo-2 | 20.5 |
| 83219_CC Well to Mod Diff | 18.6 |
| (ODO3866) | |
| 94936_Colon Adenocarcinoma | 19.5 |
| 94930_Colon | 25.3 |
| 94935_Colon Adenocarcinoma | 21.1 |
| 113468_Colon Pool | 16.8 |
| 113457_Small Intestine Pool | 13.3 |
| 113460_Stomach Pool | 8.7 |
| 113467_Bone Marrow Pool | 7.4 |
| 103371_Fetal Heart | 5.1 |
| 113451_Heart Pool | 5.3 |
| 113466_Lymph Node Pool | 20.7 |
| 103372_Fetal Skeletal Muscle | 4.9 |
| 113456_Skeletal Muscle Pool | 3.2 |
| 113459_Spleen Pool | 9.0 |
| 113462_Thymus Pool | 15.3 |
| CNS ca. (glio/astro)_U87-MG | 11.1 |
| CNS ca. (glio/astro)_U-118- | 26.5 |
| MG | |
| CNS ca. (neuro;met)_SK-N-AS | 32.3 |
| 95264_Brain astrocytoma | 14.4 |
| CNS ca. (astro)_SNB-75 | 21.4 |
| CNS ca. (glio)_SNB-19 | 15.3 |
| CNS ca. (glio)_SF-295 | 17.8 |
| 113447_Brain (Amygdala) Pool | 4.4 |
| 103382_Brain (cerebellum) | 14.2 |
| 64019-1_brain (fetal) | 13.6 |
| 113448_Brain (hippocampus) | 5.6 |
| Pool | |
| 113464_Cerebral Cortex Pool | 7.1 |
| 113449_Brain (Substantianigra) | 5.4 |
| Pool | |
| 113450_Brain (Thalamus) Pool | 9.4 |
| 103384_Brain (whole) | 8.5 |
| 113458_Spinal Cord Pool | 11.1 |
| 103375_Adrenal Gland | 7.4 |
| 113454_Pituitary gland Pool | 1.9 |
| 103397_Salivary Gland | 3.6 |
| 103369_Thyroid (female) | 7.5 |
| Pancreatic ca._CAPAN2 | 34.6 |
| 113453_Pancreas Pool | 17.5 |

TABLE 14
Panel 2D

| | Relative |
|---|---|
| | Expression (%) |
| | 2dx4tm4640t_{—} |
| Tissue Name | ag165_b1 |
| Normal Colon GENPAK 061003 | 75.4 |
| 83219 CC Well to Mod Diff (ODO3866) | 16.8 |
| 83220 CC NAT (ODO3866) | 11.0 |
| 83221 CC Gr. 2 rectosigmoid (ODO3868) | 18.6 |
| 83222 CC NAT (ODO3868) | 4.2 |
| 83235 CC Mod Diff (ODO3920) | 54.8 |
| 83236 CC NAT (ODO3920) | 14.8 |
| 83237 CC Gr. 2 ascend colon (ODO3921) | 71.6 |
| 83238 CC NAT (ODO3921) | 11.2 |
| 83241 CC from Partial Hepatectomy (ODO4309) | 52.2 |
| 83242 Liver NAT (ODO4309) | 13.2 |
| 87472 Colon mets to lung (OD04451-01) | 32.0 |
| 87473 Lung NAT (OD04451-02) | 12.2 |
| Normal Prostate Clontech A+ 6546-1 | 80.7 |
| 84140 Prostate Cancer (OD04410) | 31.0 |
| 84141 Prostate NAT (OD04410) | 18.8 |
| 87073 Prostate Cancer (OD04720-01) | 25.2 |
| 87074 Prostate NAT (OD04720-02) | 31.7 |
| Normal Lung GENPAK 061010 | 32.4 |
| 83239 Lung Met to Muscle (ODO4286) | 29.5 |
| 83240 Muscle NAT (ODO4286) | 8.4 |
| 84136 Lung Malignant Cancer (OD03126) | 48.8 |
| 84137 Lung NAT (OD03126) | 33.5 |
| 84871 Lung Cancer (OD04404) | 57.7 |
| 84872 Lung NAT (OD04404) | 23.2 |
| 84875 Lung Cancer (OD04565) | 19.9 |
| 84876 Lung NAT (OD04565) | 13.8 |
| 85950 Lung Cancer (OD04237-01) | 58.1 |
| 85970 Lung NAT (OD04237-02) | 32.2 |
| 83255 Ocular Mel Met to Liver (ODO4310) | 33.9 |
| 83256 Liver NAT (ODO4310) | 10.1 |
| 84139 Melanoma Mets to Lung (OD04321) | 29.4 |
| 84138 Lung NAT (OD04321) | 30.3 |
| Normal Kidney GENPAK 061008 | 37.0 |
| 83786 Kidney Ca, Nuclear grade 2 (OD04338) | 36.6 |
| 83787 Kidney NAT (OD04338) | 23.5 |
| 83788 Kidney Ca Nuclear grade 1/2 (OD04339) | 40.2 |
| 83789 Kidney NAT (OD04339) | 25.3 |
| 83790 Kidney Ca, Clear cell type (OD04340) | 32.0 |
| 83791 Kidney NAT (OD04340) | 23.7 |
| 83792 Kidney Ca, Nuclear grade 3 (OD04348) | 12.4 |
| 83793 Kidney NAT (OD04348) | 23.6 |
| 87474 Kidney Cancer (OD04622-01) | 16.9 |
| 87475 Kidney NAT (OD04622-03) | 5.8 |
| 85973 Kidney Cancer (OD04450-01) | 32.9 |
| 85974 Kidney NAT (OD04450-03) | 24.0 |
| Kidney Cancer Clontech 8120607 | 12.2 |
| Kidney NAT Clontech 8120608 | 7.0 |
| Kidney Cancer Clontech 8120613 | 12.0 |
| Kidney NAT Clontech 8120614 | 7.0 |
| Kidney Cancer Clontech 9010320 | 18.0 |
| Kidney NAT Clontech 9010321 | 11.9 |
| Normal Uterus GENPAK 061018 | 6.8 |
| Uterus Cancer GENPAK 064011 | 36.3 |
| Normal Thyroid Clontech A+ 6570-1 | 38.6 |
| Thyroid Cancer GENPAK 064010 | 36.3 |
| Thyroid Cancer INVITROGEN A302152 | 15.6 |
| Thyroid NAT INVITROGEN A302153 | 35.7 |
| Normal Breast GENPAK 061019 | 22.2 |
| 84877 Breast Cancer (OD04566) | 40.0 |
| 85975 Breast Cancer (OD04590-01) | 57.1 |
| 85976 Breast Cancer Mets (OD04590-03) | 54.3 |
| 87070 Breast Cancer Metastasis (OD04655-05) | 40.3 |
| GENPAK Breast Cancer 064006 | 22.8 |
| Breast Cancer Res. Gen. 1024 | 30.5 |
| Breast Cancer Clontech 9100266 | 58.1 |
| Breast NAT Clontech 9100265 | 26.6 |
| Breast Cancer INVITROGEN A209073 | 57.1 |
| Breast NAT INVITROGEN A2090734 | 23.8 |
| Normal Liver GENPAK 061009 | 8.4 |
| Liver Cancer GENPAK 064003 | 14.4 |
| Liver Cancer Research Genetics RNA 1025 | 5.4 |
| Liver Cancer Research Genetics RNA 1026 | 11.7 |
| Paired Liver Cancer Tissue Research Genetics | 6.6 |
| RNA 6004-T | |
| Paired Liver Tissue Research Genetics RNA 6004-N | 14.8 |
| Paired Liver Cancer Tissue Research Genetics | 11.6 |
| RNA 6005-T | |
| Paired Liver Tissue Research Genetics RNA 6005-N | 2.5 |
| Normal Bladder GENPAK 061001 | 53.6 |
| Bladder Cancer Research Genetics RNA 1023 | 23.6 |
| Bladder Cancer INVITROGEN A302173 | 39.7 |
| 87071 Bladder Cancer (OD04718-01) | 63.5 |
| 87072 Bladder Normal Adjacent (OD04718-03) | 33.6 |
| Normal Ovary Res. Gen. | 18.7 |
| Ovarian Cancer GENPAK 064008 | 57.1 |
| 87492 Ovary Cancer (OD04768-07) | 100.0 |
| 87493 Ovary NAT (OD04768-08) | 6.9 |
| Normal Stomach GENPAK 061017 | 35.8 |
| Gastric Cancer Clontech 9060358 | 17.6 |
| NAT Stomach Clontech 9060359 | 19.0 |
| Gastric Cancer Clontech 9060395 | 40.6 |
| NAT Stomach Clontech 9060394 | 25.7 |
| Gastric Cancer Clontech 9060397 | 49.7 |
| NAT Stomach Clontech 9060396 | 10.8 |
| Gastric Cancer GENPAK 064005 | 77.8 |

TABLE 15
Panel 4D

| | Relative |
|---|---|
| | Expression (%) |
| | 4dx4tm4605t_{—} |
| Tissue Name | ag165_a1 |
| 93768_Secondary Th1_anti-CD28/anti-CD3 | 12.3 |
| 93769_Secondary Th2_anti-CD28/anti-CD3 | 13.8 |
| 93770_Secondary Tr1_anti-CD28/anti-CD3 | 11.4 |
| 93573_Secondary Th1_resting 4-6 in IL-2 | 1.7 |
| 93572_Secondary Th2_resting day 4-6 in IL-2 | 2.4 |
| 93571_Secondary Tr1_resting day 4-6 in IL-2 | 2.3 |
| 93568_primary Th1_anti-CD28/anti-CD3 | 16.6 |
| 93569_primary Th2_anti-CD28/anti-CD3 | 13.7 |
| 93570_primary Tr1_anti-CD28/anti-CD3 | 23.2 |
| 93565_primary Th1_resting dy 4-6 in IL-2 | 19.8 |
| 93566_primary Th2_resting dy 4-6 in IL-2 | 10.4 |
| 93567_primary Tr1_resting dy 4-6 in IL-2 | 8.4 |
| 93351_CD45RA CD4 lymphocyte_anti-CD28/anti-CD3 | 8.3 |
| 93352_CD45RO CD4 lymphocyte_anti-CD28/anti-CD3 | 12.9 |
| 93251_CD8 Lymphocytes_anti-CD28/anti-CD3 | 10.6 |
| 93353_chronic CD8 Lymphocytes 2ry_resting dy 4-6 in | 12.0 |
| IL-2 | |
| 93574_chronic CD8 Lymphocytes 2ry_activated | 8.8 |
| CD3/CD28 | |
| 93354_CD4_none | 2.2 |
| 93252_Secondary Th1/Th2/Tr1_anti-CD95 CH11 | 4.7 |
| 93103_LAX cells_resting | 6.7 |
| 93788_LAX cells_IL-2 | 11.2 |
| 93787_LAX cells_IL-2 + IL-12 | 11.4 |
| 93789_LAK cells_IL-2 + IFN gamma | 16.6 |
| 93790_LAK cells_IL-2 + IL-18 | 11.0 |
| 93104_LAK cells_PMA/ionomycin and IL-18 | 5.1 |
| 93578_NK Cells IL-2_resting | 6.0 |
| 93109_Mixed Lymphocyte Reaction_Two Way MLR | 5.6 |
| 93110_Mixed Lymphocyte Reaction_Two Way MLR | 7.1 |
| 93111_Mixed Lymphocyte Reaction_Two Way MLR | 6.6 |
| 93112_Mononuclear Cells (PBMCs)_resting | 2.7 |
| 93113_Mononuclear Cells (PBMCs)_PWM | 35.9 |
| 93114_Mononuclear Cells (PBMCs)_PHA-L | 20.2 |
| 93249_Ramos (B cell)_none | 32.4 |
| 93250_Ramos (B cell)_ionomycin | 100.0 |
| 93349_B lymphocytes_PWM | 80.0 |
| 93350_B lymphoytes_CD40L and IL-4 | 16.8 |
| 92665_EOL-1 (Eosinophil)_dbcAMP differentiated | 12.6 |
| 93248_EOL-1 (Eosinophil)_dbcAMP/PMAionomycin | 6.3 |
| 93356_Dendritic Cells_none | 5.3 |
| 93355_Dendritic Cells_LPS 100 ng/ml | 2.9 |
| 93775_Dendritic Cells_anti-CD40 | 5.6 |
| 93774_Monocytes_resting | 4.1 |
| 93776_Monocytes_LPS 50 ng/ml | 1.2 |
| 93581_Macrophages_resting | 5.9 |
| 93582_Macrophages_LPS 100 ng/ml | 1.9 |
| 93098_HUVEC (Endothelial)_none | 10.5 |
| 93099_HUVEC (Endothelial)_starved | 21.7 |
| 93100_HUVEC (Endothelial)_IL-1b | 7.3 |
| 93779_HUVEC (Endothelial)_IFN gamma | 8.5 |
| 93102_HUVEC (Endothelial)_TNF alpha + IFN gamma | 6.6 |
| 93101_HUVEC (Endothelial)_TNF alpha + IL4 | 8.9 |
| 93781_HUVEC (Endothelial)_IL-11 | 6.1 |
| 93583_Lung Microvascular Endothelial Cells_none | 5.2 |
| 93584_Lung Microvascular Endothelial Cells_TNFa | 6.6 |
| (4 ng/ml) and IL 1b (1 ng/ml) | |
| 92662_Microvascular Dermal endothelium_none | 9.9 |
| 92663_Microsvasular Dermal endothelium_TNFa | 6.3 |
| (4 ng/ml) and IL 1b (1 ng/ml) | |
| 93773_Bronchial epithelium_TNFa (4 ng/ml) and IL 1b | 5.2 |
| (1 ng/ml) ** | |
| 93347_Small Airway Epithelium_none | 2.3 |
| 93348_Small Airway Epithelium_TNFa (4 ng/ml) and | 12.7 |
| IL 1b (1 ng/ml) | |
| 92668_Coronery Artery SMC_resting | 5.1 |
| 92669_Coronery Artery SMC_TNFa (4 ng/ml) and IL | 1.7 |
| 1b (1 ng/ml) | |
| 93107_astrocytes_resting | 1.7 |
| 93108_astrocytes_TNFa (4 ng/ml) and IL (1 ng/ml) | 0.9 |
| 92666_KU-812 (Basophil)_resting | 11.5 |
| 92667_KU-812 (Basophil)_PMA/ionoycin | 27.6 |
| 93579_CCD1106 (Keratinocytes)_none | 8.4 |
| 93580_CCD11O6 (Keratinocytes)_TNFa and IFNg ** | 3.2 |
| 93791_Liver Cirrhosis | 1.2 |
| 93792_Lupus Kidney | 0.8 |
| 93577_NCI-H292 | 14.8 |
| 93358_NCI-H292_IL-4 | 12.3 |
| 93360_NCI-H292_IL-9 | 18.9 |
| 93359_NCI-H292_IL-13 | 8.8 |
| 93357_NCI-H292_IFN gamma | 8.5 |
| 93777_HPAEC_- | 6.4 |
| 93778_HPAEC_IL-1 beta/TNA alpha | 5.9 |
| 93254_Normal Human Lung Fibroblast_none | 3.0 |
| 93253_Normal Human Lung Fibroblast_TNFa | 2.9 |
| (4 ng/ml) and IL-1b (1 ng/ml) | |
| 93257_Normal Human Lung Fibroblast_IL-4 | 6.2 |
| 93256_Normal Human Lung Fibroblast_IL-9 | 5.8 |
| 93255_Normal Human Lung Fibroblast_IL-13 | 5.1 |
| 93258_Normal Human Lung Fibroblast_IFN gamma | 10.0 |
| 93106_Dermal Fibroblasts CCD1070_resting | 11.7 |
| 93361_Dermal Fibroblasts CCD1070_TNF alpha | 27.6 |
| 4 ng/ml | |
| 93105_Dermal Fibroblasts CCD1070_IL-1 beta 1 ng/ml | 4.9 |
| 93772_dermal fibroblast_IFN gamma | 3.6 |
| 93771_dermal fibroblast_IL-4 | 5.7 |
| 93260_IBD Colitis 2 | 1.1 |
| 93261_IBD Crohns | 0.7 |
| 735010_Colon_normal | 5.4 |
| 735019_Lung_none | 5.8 |
| 64028-1_Thymus_none | 4.7 |
| 64030-1_Kidney_none | 18.0 |

TABLE 16
Panel 4.1D

| | Relative |
|---|---|
| | Expression (%) |
| | 4.1dx4tm6228f |
| Tissue Name | _ag3750_a1 |
| 93768_Secondary Th1_anti-CD28/anti-CD3 | 62.5 |
| 93769_Secondary Th2_anti-CD28/anti-CD3 | 57.2 |
| 93770_Secondary Tr1_anti-CD28/anti-CD3 | 49.3 |
| 93573_Secondary Th1_resting day 4-6 in IL-2 | 6.7 |
| 93572_Secondary Th2_resting day 4-6 in IL-2 | 8.9 |
| 93571_Secondary Tr1_resting day 4-6 in IL-2 | 11.4 |
| 93568_primary Th1_anti-CD28/anti-CD3 | 49.3 |
| 93569_primary Th2_anti-CD28/anti-CD3 | 51.6 |
| 93570_primary Tr1_anti-CD28/anti-CD3 | 65.6 |
| 93565_primary Th1_resting dy 4-6 in IL-2 | 14.6 |
| 93566_primary Th2_resting dy 4-6 in IL-2 | 8.6 |
| 93567_primary Tr1_resting dy 4-6 in IL-2 | 17.5 |
| 93351_CD45RA CD4 lymphocyte_anti-CD28/anti-CD3 | 37.8 |
| 93352_CD45RO CD4 lymphocyte_anti-CD28/anti-CD3 | 79.7 |
| 93251_CD8 Lymphocytes_anti-CD28/anti-CD3 | 59.0 |
| 93353_chronic CD8 Lymphocytes 2ry_resting dy 4-6 in | 66.8 |
| IL-2 | |
| 93574_chronic CD8 Lymphocytes 2ry_activated | 23.0 |
| CD3/CD28 | |
| 93354_CD4_none | 12.7 |
| 93252_Secondary Th1/Th2/Tr1_anti-CD95 CH11 | 20.7 |
| 93103_LAK cells_resting | 28.9 |
| 93788_LAK cells_IL-2 | 37.4 |
| 93787_LAK cells_IL-2 + IL-12 | 29.5 |
| 93789_LAK cells_IL-2 + IFN gamma | 22.5 |
| 93790_LAK cells_IL-2 + IL-18 | 24.0 |
| 93104_LAK cells_PMA/ionomycin and IL-18 | 27.2 |
| 93578_NK Cells IL-2_resting | 40.5 |
| 93109_Mixed Lymphocyte Reaction_Two Way MLR | 23.4 |
| 93110_Mixed Lymphocyte Reaction_Two Way MLR | 32.9 |
| 93111_Mixed Lymphocyte Reaction_Two Way MLR | 21.9 |
| 93112_Mononuclear Cells (PBMCs)_resting | 16.6 |
| 93113_Mononuclear Cells (PBMCs)_PWM | 27.1 |
| 93114_Mononuclear Cells (PBMCs)_PHA-L | 45.3 |
| 93249_Ramos (B cell)_none | 100.0 |
| 93250_Ramos (B cell)_ionomycin | 88.8 |
| 93349_B lymphocytes_PWM | 53.9 |
| 93350_B lymphoytes_CD40L and IL-4 | 40.9 |
| 92665_EOL-1 (Eosinophil)_dbcAMP differentiated | 73.9 |
| 93248_EOL-1 (Eosinophil)_dbcAMP/PMAionomycin | 26.5 |
| 93356_Dendritic Cells_none | 22.6 |
| 93355_Dendritic Cells_LPS 100 ng/ml | 12.8 |
| 93775_Dendritic Cells_anti-CD40 | 23.2 |
| 93774_Monocytes_resting | 18.4 |
| 93776_Monocytes_LPS 50 ng/ml | 9.3 |
| 93581_Macrophages_resting | 20.4 |
| 93582_Macrophages_LPS 100 ng/ml | 7.4 |
| 93098_HUVEC (Endothelial)_none | 26.0 |
| 93099_HUVEC (Endothelial)_starved | 39.1 |
| 93100_HUVEC (Endothelial)_IL-1b | 44.8 |
| 93779_HUVEC (Endothelial)_IFN gamma | 45.0 |
| 93102_HUVEC (Endothelial)_TNF alpha + IFN gamma | 26.9 |
| 93101_HUVEC (Endothelial)_TNF alpha + IL4 | 31.7 |
| 93781_HUVEC (Endothelial)_IL-11 | 32.7 |
| 93583_Lung Microvascular Endothelial Cells_none | 30.1 |
| 93584_Lung Microvascular Endothelial Cells_TNFa | 23.4 |
| (4 ng/ml) and IL 1b (1 ng/ml) | |
| 92662_Microvascular Dermal endothelium_none | 16.7 |
| 92663_Microsvasular Dermal endothelium_TNFa | 12.4 |
| (4 ng/ml) and IL 1b (1 ng/ml) | |
| 93773_Bronchial epithelium_TNFa (4 ng/ml) and IL 1b | 21.7 |
| (1 ng/ml) ** | |
| 93347_Small Airway Epithelium_none | 6.1 |
| 93348_Small Airway Epithelium_TNFa (4 ng/ml) and | 12.8 |
| IL 1b (1 ng/ml) | |
| 92668_Coronery Artery SMC_resting | 15.6 |
| 92669_Coronery Artery SMC_TNFa (4 ng/ml) and IL | 9.7 |
| 1b (1 ng/ml) | |
| 93107_astrocytes_resting | 5.4 |
| 93108_astrocytes_TNFa (4 ng/ml) and IL 1b (1 ng/ml) | 3.1 |
| 92666_KU-812 (Basophil)_resting | 57.9 |
| 92667_KU-812 (Basophil)_PMA/ionoycin | 81.2 |
| 93579_CCD1106 (Keratinocytes)_none | 27.9 |
| 93580_CCD1106 (Keratinocytes)_TNFa and IFNg ** | 18.0 |
| 93791_Liver Cirrhosis | 9.4 |
| 93577_NCI-H292 | 23.0 |
| 93358_NCI-H292_IL-4 | 29.1 |
| 93360_NCI-H292_IL-9 | 40.4 |
| 93359_NCI-H292_IL-13 | 31.9 |
| 93357_NCI-H292_IFN gamma | 23.5 |
| 93777_HPAEC_- | 25.9 |
| 93778_HPAEC_IL-1 beta/TNA alpha | 27.3 |
| 93254_Normal Human Lung Fibroblast_none | 15.9 |
| 93253_Normal Human Lung Fibroblast_TNFa | 12.9 |
| (4 ng/ml) and IL-1b (1 ng/ml) | |
| 93257_Normal Human Lung Fibroblast_IL-4 | 44.8 |
| 93256_Normal Human Lung Fibroblast_IL-9 | 20.6 |
| 93255_Normal Human Lung Fibroblast_IL-13 | 15.7 |
| 93258_Normal Human Lung Fibroblast_IFN gamma | 25.2 |
| 93106_Dermal Fibroblasts CCD1070_resting | 28.6 |
| 93361_Dermal Fibroblasts CCD1070_TNF alpha | 54.9 |
| 4 ng/ml | |
| 93105_Dermal Fibroblasts CCD1070_IL-1 beta 1 ng/ml | 14.4 |
| 93772_dermal fibroblast_IFN gamma | 17.2 |
| 93771_dermal fibroblast_IL-4 | 18.6 |
| 93892_Dermal fibroblasts_none | 11.8 |
| 99202_Neutrophils_TNFa + LPS | 2.3 |
| 99203_Neutrophils_none | 3.9 |
| 735010_Colon_normal | 5.4 |
| 735019_Lung_none | 15.7 |
| 64028-1_Thymus_none | 54.5 |
| 64030-1_Kidney_none | 14.9 |

TABLE 17
CNS_neurodegeneration_panel_v1.0

| | Relative |
|---|---|
| | Expression (%) |
| | tm6945t_{—} |
| Tissue Name | ag165_a1 |
| AD 1 Hippo | 14.3 |
| AD 2 Hippo | 23.1 |
| AD 3 Hippo | 11.8 |
| AD 4 Hippo | 8.3 |
| AD 5 hippo | 82.8 |
| AD 6 Hippo | 56.7 |
| Control 2 Hippo | 32.3 |
| Control 4 Hippo | 12.4 |
| Control (Path) 3 Hippo | 10.1 |
| AD 1 Temporal Ctx | 26.4 |
| AD 2 Temporal Ctx | 27.1 |
| AD 3 Temporal Ctx | 9.7 |
| AD 4 Temporal Ctx | 23.9 |
| AD 5 Inf Temporal Ctx | 68.4 |
| AD 5 Sup Temporal Ctx | 39.8 |
| AD 6 Inf Temporal Ctx | 69.9 |
| AD 6 Sup Temporal Ctx | 52.9 |
| Control 1 Temporal Ctx | 9.9 |
| Control 2 Temporal Ctx | 24.3 |
| Control 3 Temporal Ctx | 14.5 |
| Control 4 Temporal Ctx | 9.3 |
| Control (Path) 1 Temporal Ctx | 39.2 |
| Control (Path) 2 Temporal Ctx | 24.4 |
| Control (Path) 3 Temporal Ctx | 7.4 |
| Control (Path) 4 Temporal Ctx | 24.7 |
| AD 1 Occipital Ctx | 20.6 |
| AD 2 Occipital Ctx (Missing) | 0.0 |
| AD 3 Occipital Ctx | 9.4 |
| AD 4 Occipital Ctx | 25.6 |
| AD 5 Occipital Ctx | 100.0 |
| AD 6 Occipital Ctx | 35.0 |
| Control 1 Occipital Ctx | 9.2 |
| Control 2 Occipital Ctx | 32.0 |
| Control 3 Occipital Ctx | 13.2 |
| Control 4 Occipital Ctx | 30.3 |
| Control (Path) 1 Occipital Ctx | 55.9 |
| Control (Path) 2 Occipital Ctx | 12.5 |
| Control (Path) 3 Occipital Ctx | 7.0 |
| Control (Path) 4 Occipital Ctx | 19.8 |
| Control 1 Parietal Ctx | 11.4 |
| Control 2 Parietal Ctx | 40.7 |
| Control 3 Parietal Ctx | 11.8 |
| Control (Path) 1 Parietal Ctx | 38.3 |
| Control (Path) 2 Parietal Ctx | 21.8 |
| Control (Path) 3 Parietal Ctx | 8.2 |
| Control (Path) 4 Parietal Ctx | 29.1 |

Panel 1 Summary: Ag185b The AC010175_A.0.1 gene encodes a protein with homology to prothymosin alpha. Expression of the AC010175_A.0.1 gene is highest in a sample derived from a melanoma cell line (LOX INWI) (CT=20). In addition, there is also substantial expression of this gene in samples derived from thymus, a lung cancer cell line (SHP-77) and a colon cancer cell line (HCT-116). Thus, this gene could be used to distinguish these samples from other samples in the panel. Moreover, therapeutic modulation of the expression or activity of the AC010175_A.0.1 gene product, through the use of small molecule drugs, antibodies or protein therapeutics might be of benefit for the treatment of melanoma, lung or colon cancer. However, there also appears to be widespread but lower expression of this gene in the remainder of the samples in the panel, suggesting a general role in cellular function.

Among CNS samples, AC010175_A.0.1 gene expression is highest in cerebral cortex and spinal cord (CT=22-23) with lower expression in thalamus, substantia nigra, hippocampus, cerebellum and amygdala. Please see CNS_neurodegeneration_panel_v1.0 for a description of the potential utility of this gene in treating CNS disorders.

The AC010175_A.0.1 gene is also expressed at high levels in metabolic tissues including adipose, pancreas, adrenal gland, pituitary gland, thyroid, adult/fetal heart, and adult/fetal skeletal muscle (CTs=22-26). Therefore, this gene product may be involved in the pathogenesis and/or treatment of disease in any or all of these tissues.

Panel 1.3D Summary: Ag165 Expression of the AC010175_A.0.1 gene is highest in ovary, thymus and liver adenocarcinoma (CT=23). Thus, the expression of this gene could be used to distinguish the above-mentioned tissues from other tissues.

Among CNS samples, AC010175_A.0.1 gene expression is highest in cerebral cortex (CT=23) with lower expression in spinal cord, thalamus, substantia nigra, hippocampus, cerebellum and amygdala (CTs=25-27). Please see CNS_neurodegeneration_panel_v1.0 for a description of the potential utility of this gene in treating CNS disorders.

The AC010175_A.0.1 gene is also expressed at high levels in metabolic tissues including adipose, pancreas, adrenal gland, pituitary gland, thyroid, adult/fetal heart, and adult/fetal skeletal muscle (CTs=24-28). Therefore, this gene product may be involved in the pathogenesis and/or treatment of disease in any or all of these tissues.

General_screening_panel_v1.4 Summary: Ag3750 Expression of the AC010175_A.0.1 gene is highest in two breast cancer cell lines (CT=23). In general, this gene is expressed at higher levels in cancer cell lines than in normal tissues. For example, expression of the AC010175_A.0.1 gene is associated with breast cancer cell lines, melanoma cell lines and colon cancer cell lines. Thus, the expression of this gene could be used to distinguish breast cancer, colon cancer or melanoma cell lines from other samples. Moreover, therapeutic modulation of the expression or activity of the AC010175_A.0.1 gene, through the use of small molecule drugs, antibodies or protein therapeutics might be of benefit for the treatment of melanoma, breast or colon cancer.

The AC010175_A.0.1 gene is highly expressed throughout the CNS including in cerebral cortex, spinal cord, thalamus, substantia nigra, hippocampus, cerebellum and amygdala (CTs=25-27). Please see CNS_neurodegeneration_panel_v1.0 for a description of the potential utility of this gene in treating CNS disorders.

The AC010175_A.0.1 gene is also expressed at high levels in metabolic tissues including adipose, pancreas, adrenal gland, pituitary gland, thyroid, adult/fetal heart, and adult/fetal skeletal muscle (CTs=25-29). Therefore, this gene product may be involved in the pathogenesis and/or treatment of disease in any or all of these tissues.

Panel 2D Summary: Ag165 Expression of the AC010175_A.0.1 gene is highest in a sample derived from an ovarian cancer (CT=22.7) on this panel. Overall there appears to be a consistent association with the higher expression of this gene in cancerous tissues. The AC010175_A.0.1 gene is overexpressed in cancerous tissue from stomach, ovary, lung and colon when compared to normal adjacent tissue. Thus, the expression of this gene could be used as a marker to distinguish between stomach, ovarian, lung or colon cancers and their respective normal adjacent tissues and could be of benefit for the diagnosis or prognostication of these diseases. Moreover, therapeutic modulation of the expression or activity of the AC010175_A.0.1 gene, through the use of small molecule drugs, antibodies or protein therapeutics might be beneficial for the treatment of stomach, ovarian, lung or colon cancer.

Panel 4D Summary: Ag165 Expression of the AC010175_A.0.1 gene is ubiquitous throughout Panel 4D. However, the gene is expressed preferentially at high levels in B cells. The AC010175_A.0.1 gene is highly upregulated in ionomycin-treated Ramos B cells (CT=20.1) as well as in primary B cells after activation with PWM. This gene product is homologous to the nuclear protein prothymosin alpha that is thought to play a role in proliferation and chromatin remodeling. Therefore, small molecule or antibody therapeutics designed against the AC010175_A.0.1 protein might be useful for the treatment of lymphoproliferative disorders, autoimmune disease and B cell lymphomas.

Panel 4.1D Summary: Ag3750 Expression of the AC010175_A.0.1 gene is ubiquitous throughout Panel 4.1 D. However, highest expression of this gene is seen in the Ramos B cell line (activated or not; CTs=23) as well as in activated basophils and eosinophils. Expression of the AC010175_A.0.1 gene is highest in Ramos B cells irrespective of treatment. This gene product is homologous to the nuclear protein prothymosin alpha that is thought to play a role in proliferation and chromatin remodeling. Therefore, small molecule or antibody therapeutics designed against the ACO 10175_A.0.1 protein might be useful for the treatment of lymphoproliferative disorders, autoimmune disease and B cell lymphomas

CNS_neurodegeneration_panel_v1.0 Summary: Ag165 Expression of the AC010175_A.0.1 gene is highest in the occipital cortex from an Alzheimer's disease patient (CT=24. 1). Furthermore, this gene is slightly (but detectably) increased in the temporal cortex of Alzheimer's disease patients (1.3-fold increase), this increase is detectable after correcting CT values for overall mRNA quality. Prothymosin alpha is the immature form of thymosin alpha. Thymosin alpha is critical for maintenance of levels of both nerve growth factor (NGF) and its receptor (p75NGFr); early thymectomy decreases levels of both of these proteins, which are partially restored by injection of thymosin alpha. NGF in turn is critical for the formation and maintenance of basal cholinergic neurons, a population of neurons that specifically degenerate in Alzheimer's disease (AD). The increase in levels of this protein seen in AD may be an attempt on the part of the CNS to compensate for the loss of cholinergic neurons. Therefore, therapeutic modulation of the AC009485_A gene or its protein product in Alzheimer's disease may be useful in slowing neurodegeneration of cholinergic neurons, especially when used in conjunction with acetylcholine esterase inhibitors (a standard of AD therapy). (Turrini et al., A role of the thymus and thymosin-alpha1 in brain NGF levels and NGF receptor expression. J. Neuroimmunol. 82:64-72, 1998; Turrini and Aloe, Evidence that endogenous thymosin alpha-1 is present in the rat central nervous system. Neurochem. Int. 35:463-70. 1999; Fahnestock et al., The precursor pro-nerve growth factor is the predominant form of nerve growth factor in brain and is increased in alzheimer's disease. Mol. Cell. Neurosci. 18:210-220. 2001.)

C. Clone Identification No: AC009533_A (PTMA 4)

Expression of gene AC009533_A was assessed using the primer-probe sets Ag1855 described in Table 18. Results from RTQ-PCR runs are shown in Tables 19.

TABLE 18
Probe Name Ag1855

| | | SEQ ID |
|---|---|---|
| Primers | Sequences | NO: |
| Forward | 5′-AGATGTCAGACGCAGCCGTA-3′ | 36 |
| Probe | TET-5′-CAGCTCCGAAATCACCACCGAGGAC-3′- | 37 |
| | TAMRA | |
| Reverse | 5′-TCCACAACTTCCTTCTTCTCCTTT-3′ | 38 |

TABLE 19

| | % Rel. | % Rel. | | % Rel. | % Rel. |
|---|---|---|---|---|---|
| | Expr. | Expr. | | Expr. | Expr. |
| Tissue_Name | tm381t | tm336t | Tissue_Name | tm381t | tm336t |
| Endothelial cells | 4.3 | 1.5 | Kidney (fetal) | 37.6 | 39.0 |
| Endothelial cells (treated) | 15.4 | 3.3 | Renal Ca. 786-0 | 8.8 | 2.9 |
| Pancreas | 20.2 | 20.6 | Renal Ca. A498 | 0.9 | 0.6 |
| Pancreatic ca. CAPAN 2 | 22.9 | 22.2 | Renal Ca. RXF 393 | 1.2 | 0.6 |
| Adipose | 44.4 | 55.9 | Renal Ca. ACHN | 2.8 | 2.6 |
| Adrenal gland | 6.7 | 2.5 | Renal Ca. UO-31 | 0.4 | 0.6 |
| Thyroid | 30.4 | 51.1 | Renal Ca. TK-10 | 4.6 | 8.1 |
| Salavary gland | 5.2 | 2.2 | Liver | 21.9 | 11.6 |
| Pituitary gland | 3.6 | 8.4 | Liver (fetal) | 4.7 | 6.0 |
| Brain (fetal) | 2.4 | 4.1 | Liver Ca. (hepatoblast) HepG2 | 1.5 | 0.6 |
| Brain (whole) | 14.1 | 11.7 | Lung | 10.7 | 16.0 |
| Brain (amygdala) | 2.3 | 3.5 | Lung (fetal) | 13.2 | 44.4 |
| Brain (cerebellum) | 37.4 | 28.5 | Lung Ca. (small cell) LX-1 | 27.4 | 27.9 |
| Brain (hippocampus) | 6.8 | 8.3 | Lung Ca. (small cell) NCI- | 5.3 | 3.3 |
| | | | H69 | | |
| Brain (substantia nigra) | 22.4 | 15.0 | Lung Ca. (s.cell var.) SHP-77 | 59.5 | 65.5 |
| Brain (thalamus) | 12.9 | 12.9 | Lung Ca. (large cell)NCI-H460 | 14.2 | 25.4 |
| Brain (hypothalamus) | 1.9 | 8.6 | Lung Ca. (non-sm. cell) A549 | 3.3 | 5.0 |
| Spinal cord | 10.1 | 4.2 | Lung Ca. (non-s.cell) NCI-H23 | 9.9 | 6.8 |
| CNS Ca. (glio/astro) U87-MG | 3.0 | 0.6 | Lung ca (non-s.cell) HOP-62 | 0.8 | 0.6 |
| CNS Ca. (glio/astro) U-118-MG | 2.5 | 1.9 | Lung Ca. (non-s.d) NCI-H522 | 19.2 | 26.8 |
| CNS Ca. (astro) SW1783 | 1.2 | 0.6 | Lung Ca. (squam.) SW 900 | 27.0 | 33.9 |
| CNS ca.* (neuro; met) SK-N-AS | 29.1 | 33.9 | Lung Ca. (squam.) NCI-H596 | 23.8 | 37.4 |
| CNS Ca. (astro) SF-539 | 1.0 | 0.6 | Mammary gland | 25.4 | 34.4 |
| CNS Ca. (astro) SNB-75 | 1.4 | 0.6 | Breast ca.* (p1. effusion) | 50.0 | 66.0 |
| | | | MCF-7 | | |
| CNS Ca. (glio) SNB-19 | 4.3 | 5.9 | Breast ca.* (pl.ef) MDA-MB- | 3.1 | 0.8 |
| | | | 231 | | |
| CNS Ca. (glio) U251 | 0.7 | 0.8 | Breast ca.* (p1. effusion) | 13.7 | 12.2 |
| | | | T47D | | |
| CNS Ca. (glio) SF-295 | 0.5 | 1.5 | Breast Ca. BT-549 | 40.1 | 38.7 |
| Heart | 3.6 | 1.3 | Breast ca. MDA-N | 13.1 | 25.4 |
| Skeletal muscle | 0.0 | 0.6 | Ovary | 33.2 | 23.0 |
| Bone marrow | 13.3 | 26.4 | Ovarian Ca. OVCAR-3 | 4.4 | 4.4 |
| Thymus | 66.0 | 100.0 | Ovarian Ca. OVCAR-4 | 2.4 | 2.4 |
| Spleen | 14.7 | 25.0 | Ovarian Ca. OVCAR-5 | 12.2 | 27.0 |
| Lymph node | 27.6 | 46.7 | Ovarian Ca. OVCAR-8 | 12.3 | 17.2 |
| Colon (ascending) | 29.3 | 27.4 | Ovarian Ca. IGROV-1 | 1.3 | 0.6 |
| Stomach | 12.9 | 19.5 | Ovarian ca.* (ascites) SK-OV- | 11.7 | 21.6 |
| | | | 3 | | |
| Small intestine | 18.4 | 25.2 | Uterus | 9.7 | 11.6 |
| Colon ca. 5W480 | 3.1 | 1.4 | Plancenta | 33.5 | 33.7 |
| Colon ca.* (SW480 met)SW620 | 11.8 | 17.1 | Prostate | 14.6 | 15.5 |
| Colon ca. HT29 | 14.1 | 40.1 | Prostate ca.* (bone met)PC-3 | 20.0 | 16.2 |
| Colon Ca. HCT-116 | 64.6 | 82.9 | Testis | 22.2 | 22.5 |
| Colon Ca. CaCo-2 | 7.6 | 7.8 | Melanoma Hs688(A).T | 1.1 | 0.6 |
| Colon ca. HCT-15 | 13.2 | 13.5 | Melanoma* (met) Hs688(B).T | 0.1 | 0.6 |
| Colon Ca. HCC-2998 | 9.9 | 5.1 | Melanoma UACC-62 | 0.0 | 0.6 |
| Gastric ca.* (liver met) NCI-N87 | 26.2 | 40.3 | Melanoma M14 | 6.8 | 8.4 |
| Bladder | 18.7 | 28.7 | Melanoma LOX IMVI | 100.0 | 76.8 |
| Trachea | 27.6 | 33.0 | Melanoma* (met) SK-MEL-5 | 3.3 | 10.0 |
| Kidney | 10.7 | 7.5 | Melanoma SK-MEL-28 | 9.5 | 6.0 |

The Table above shows that clone AC009533_A is highly expressed in many normal and cancer cell lines. It is highly expressed especially in melanoma LOX IMVI, breast ca.* (pl. effusion) MCF-7, lung ca. (s.cell var.) SHP-77, and colon ca. HCT-116, as well as in normal thymus cells.

D. Clone Identification No: AL121585_A (PTMA 5)

Expression of gene AL121585_A was assessed using the primer-probe set Ag1091, described in Table 20. Results from RTQ-PCR runs are shown in Table 21.

TABLE 20
Probe Name Ag1091

| | | | | Start | SEQ ID |
|---|---|---|---|---|---|
| Primers | Sequences | TM | Length | Position | NO: |
| Forward | 5′-TGCCTATACCAAGAAGCAGAAG-3′ | 58.7 | 22 | 424 | 39 |
| Probe | FAM-5- | 64.7 | 26 | 447 | 40 |
| | CCAACAAGGATGACTAGACAGCAAAA- | | | | |
| | 3′-TAMRA | | | | |
| Reverse | 5′-TGAATAGGTCACCCTCCTAACA-3′ | 58.6 | 22 | 480 | 41 |

TABLE 21
Panel 1.2

| | Relative |
|---|---|
| | Expression (%) |
| | 1.2tm1182f_{—} |
| Tissue Name | ag1091 |
| Endothelial cells | 0.0 |
| Heart (fetal) | 0.0 |
| Pancreas | 9.2 |
| Pancreatic ca. CAPAN 2 | 29.3 |
| Adrenal Gland (new lot*) | 0.0 |
| Thyroid | 2.1 |
| Salavary gland | 49.7 |
| Pituitary gland | 4.8 |
| Brain (fetal) | 2.6 |
| Brain (whole) | 0.8 |
| Brain (amygdala) | 0.4 |
| Brain (cerebellum) | 0.0 |
| Brain (hippocampus) | 0.8 |
| Brain (thalamus) | 0.0 |
| Cerebral Cortex | 1.0 |
| Spinal cord | 0.0 |
| CNS ca. (glio/astro) U87-MG | 0.0 |
| CNS ca. (glio/astro) U-118-MG | 0.0 |
| CNS ca. (astro) SW1783 | 0.0 |
| CNS ca.* (neuro; met) SK-N-AS | 0.0 |
| CNS ca. (astro) SF-539 | 0.0 |
| CNS ca. (astro) SNB-75 | 0.0 |
| CNS ca. (glio) SNB-19 | 0.0 |
| CNS ca. (glio) U251 | 0.0 |
| CNS ca. (glio) SF-295 | 0.0 |
| Heart | 0.0 |
| Skeletal Muscle (new lot*) | 0.0 |
| Bone marrow | 0.0 |
| Thymus | 0.0 |
| Spleen | 0.0 |
| Lymph node | 0.0 |
| Colorectal | 4.0 |
| Stomach | 93.3 |
| Small intestine | 32.5 |
| Colon ca. SW480 | 0.2 |
| Colon ca.* (SW480 met)SW620 | 0.0 |
| Colon ca. HT29 | 1.6 |
| Colon ca. HCT-116 | 0.0 |
| Colon ca. CaCo-2 | 10.2 |
| 83219 CC Well to Mod Diff (OD03866) | 3.6 |
| Colon ca. HCC-2998 | 98.6 |
| Gastric ca.* (liver met) NCI-N87 | 68.3 |
| Bladder | 21.3 |
| Trachea | 15.5 |
| Kidney | 20.6 |
| Kidney (fetal) | 11.0 |
| Renal ca. 786-0 | 0.0 |
| Renal ca. A498 | 0.0 |
| Renal ca. RXF 393 | 0.0 |
| Renal ca. ACHN | 0.0 |
| Renal ca. UO-31 | 0.0 |
| Renal ca. TK-10 | 2.9 |
| Liver | 0.0 |
| Liver (fetal) | 0.0 |
| Liver ca. (hepatoblast) HepG2 | 0.0 |
| Lung | 7.4 |
| Lung (fetal) | 5.0 |
| Lung ca. (small cell) LX-1 | 2.2 |
| Lung ca. (small cell) NCI-H69 | 100.0 |
| Lung ca. (s.cell var.) SHP-77 | 0.0 |
| Lung ca. (large cell)NCI-H460 | 0.0 |
| Lung ca. (non-sm. cell) A549 | 0.6 |
| Lung ca. (non-s.cell) NCI-H23 | 0.0 |
| Lung ca (non-s.cell) HOP-62 | 0.0 |
| Lung ca. (non-s.cl) NCI-H522 | 0.0 |
| Lung ca. (squam.) SW 900 | 39.5 |
| Lung ca. (squam.) NCI-H596 | 29.9 |
| Mammary gland | 10.7 |
| Breast ca.* (p1. effusion) MCF-7 | 2.8 |
| Breast ca.* (p1.ef) MDA-MB-231 | 0.0 |
| Breast ca.* (p1. effusion) T47D | 43.5 |
| Breast ca. BT-549 | 0.0 |
| Breast ca. MDA-N | 0.0 |
| Ovary | 0.0 |
| Ovarian ca. OVCAR-3 | 18.2 |
| Ovarian ca. OVCAR-4 | 8.4 |
| Ovarian ca. OVCAR-5 | 3.7 |
| Ovarian ca. OVCAR-8 | 0.0 |
| Ovarian ca. IGROV-1 | 0.0 |
| Ovarian ca.* (ascites) SK-OV-3 | 2.8 |
| Uterus | 1.4 |
| Placenta | 1.5 |
| Prostate | 86.5 |
| Prostate ca.* (bone met)PC-3 | 0.0 |
| Testis | 3.7 |
| Melanoma Hs688(A).T | 0.0 |
| Melanoma* (met) Hs688(B).T | 0.0 |
| Melanoma UACC-62 | 0.0 |
| Melanoma M14 | 1.5 |
| Melanoma LOX IMVI | 0.0 |
| Melanoma* (met) SK-MEL-5 | 0.0 |

Panel 1.2 Summary: Ag1091 The AL121585_A gene encodes a protein with homology to prothymosin alpha. In one experiment with this probe/primer set, expression of the AL121585_A gene is highest in a sample derived from lung cancer cell line NCI-H69 (CT=30.5). Overall there is also substantial expression in normal prostate and stomach tissues as well as in gastric and colon cancer cell lines. Thus, expression of the AL121585_A gene could be used to distinguish these samples from other samples.

In another experiment with the same probe/primer set, expression of the AL121585_A gene was low/undetectable (CTs>35) across the samples on this panel (data not shown).

Panel 4D Summary: Ag1091 Expression of the AL121585_A gene is low/undetectable (CTs>35) across the samples on this panel (data not shown).

E. Clone Identification No: AC010175 (PTMA 6)

Expression of gene AC010175 was assessed using the primer-probe set Ag165, described in Table 22. Results from RTQ-PCR runs are shown in Table 23.

TABLE 22
Probe Name Ag165

| | | SEQ ID |
|---|---|---|
| Primers | Sequences | NO: |
| Forward | 5′-ATGTCAGACGCAGCCGTAGA-3′ | 42 |
| Probe | TET-5′-ACCAGCTCCGAAATCACCACCGAG-3′- | 43 |
| | TAMRA | |
| Reverse | 5′-CTTCCACAACTTCCTTCTTCTCCT-3′ | 44 |

TABLE 23

| | % Rel. |
|---|---|
| Tissue_Name | Expr. |
| Endothelial cells | 11.7 |
| Endothelial cells (treated) | 15.2 |
| Pancreas | 24.2 |
| Pancreatic ca. CAPAN 2 | 34.6 |
| Adipose | 37.6 |
| Adrenal gland | 25.9 |
| Thyroid | 34.9 |
| Salavary gland | 16.8 |
| Pituitary gland | 11.1 |
| Brain (fetal) | 17.3 |
| Brain (whole) | 36.1 |
| Brain (amygdala) | 14.7 |
| Brain (cerebellum) | 50.4 |
| Brain (hippocampus) | 23.3 |
| Brain (substantia nigra) | 33.2 |
| Brain (thalamus) | 21.8 |
| Brain (hypothalamus) | 8.6 |
| Spinal cord | 16.0 |
| CNS ca. (glio/astro) U87-MG | 7.4 |
| CNS ca. (glio/astro) U-118-MG | 8.4 |
| CNS ca. (astro) SW1783 | 6.6 |
| CNS ca.* (neuro; met) SK-N-AS | 24.3 |
| CNS ca. (astro) SF-539 | 7.3 |
| CNS ca. (astro) SNB-75 | 9.0 |
| CNS ca. (glio) SNB-19 | 13.9 |
| CNS ca. (glio) U251 | 7.4 |
| CNS ca. (glio) SF-295 | 7.1 |
| Heart | 10.1 |
| Skeletal muscle | 3.0 |
| Bone marrow | 24.2 |
| Thymus | 74.2 |
| Spleen | 22.9 |
| Lymph node | 37.6 |
| Colon (ascending) | 28.1 |
| Stomach | 19.6 |
| Small intestine | 21.6 |
| Colon ca. SW480 | 9.2 |
| Colon ca.* (SW480 met)SW620 | 17.7 |
| Colon ca. HT29 | 27.6 |
| Colon ca. HCT-116 | 0.0 |
| Colon ca. CaCo-2 | 17.6 |
| Colon ca. HCT-15 | 19.9 |
| Colon ca. HCC-2998 | 20.6 |
| Gastric ca.* (liver met) NCI-N87 | 42.9 |
| Bladder | 28.1 |
| Trachea | 31.0 |
| Kidney | 18.8 |
| Kidney (fetal) | 51.1 |
| Renal ca. 786-0 | 12.9 |
| Renal ca. A498 | 4.7 |
| Renal ca. RXF 393 | 4.7 |
| Renal ca. ACHN | 13.6 |
| Renal ca. UO-31 | 6.0 |
| Renal ca. TK-10 | 13.9 |
| Liver | 26.8 |
| Liver (fetal) | 15.7 |
| Liver ca. (hepatoblast) HepG2 | 5.1 |
| Lung | 13.9 |
| Lung (fetal) | 29.5 |
| Lung ca. (small cell) LX-1 | 24.2 |
| Lung ca. (small cell) NCI-H69 | 13.2 |
| Lung ca. (s.cell var.) SHP-77 | 0.0 |
| Lung ca. (large cell)NCI-H460 | 0.0 |
| Lung ca. (non-sm. cell) A549 | 9.6 |
| Lung ca. (non-s.cell) NCI-H23 | 13.3 |
| Lung ca (non-s.cell) HOP-62 | 7.1 |
| Lung ca. (non-s.cl) NCI-H522 | 28.5 |
| Lung ca. (squam.) SW 900 | 37.6 |
| Lung ca. (squam.) NCI-H596 | 27.6 |
| Mammary gland | 27.4 |
| Breast ca.* (p1. effusion) MCF-7 | 50.4 |
| Breast ca.* (p1.ef) MDA-MB-231 | 11.5 |
| Breast ca.* (p1. effusion) T47D | 27.4 |
| Breast ca. BT-549 | 0.0 |
| Breast ca. MDA-N | 23.0 |
| Ovary | 23.5 |
| Ovarian ca. OVCAR-3 | 8.8 |
| Ovarian ca. OVCAR-4 | 7.3 |
| Ovarian ca. OVCAR-5 | 17.4 |
| Ovarian ca. OVCAR-8 | 23.5 |
| Ovarian ca. IGROV-1 | 8.4 |
| Ovarian ca.* (ascites) SK-OV-3 | 19.8 |
| Uterus | 17.1 |
| Placenta | 34.9 |
| Prostate | 21.6 |
| Prostate ca.* (bone met)PC-3 | 0.0 |
| Testis | 26.6 |
| Melanoma Hs688(A).T | 8.9 |
| Melanoma* (met) Hs688(B).T | 5.3 |
| Melanoma UACC-62 | 1.7 |
| Melanoma M14 | 19.2 |
| Melanoma LOX IMVI | 100.0 |
| Melanoma* (met) SK-MEL-5 | 13.6 |
| Melanoma SK-MEL-28 | 19.3 |

It is seen from the Table above that clone AC010175 is expressed in most normal and cancer cells assayed. It is especially prominent in Melanoma LOX IMVI and thymus.

F. Clone Identification No: AC010784-1/CG53228-01 (PTMA 7)

Expression of gene AC010784-1 was assessed using the primer-probe set Ag3076, described in Table 24. Results from RTQ-PCR run are shown in Table 25.

TABLE 24
Probe Name Ag3076

| | | | | Start | SEQ ID |
|---|---|---|---|---|---|
| Primers | Sequences | TM | Length | Position | NO: |
| Forward | 5′-AGGTGGTCTTCACACACAGG-3′ | 58.6 | 20 | 162 | 45 |
| Probe | FAM-5′- | 69.2 | 26 | 187 | 46 |
| | CAGGTCCCCATCTTCTTCAGCTTCAG- | | | | |
| | 3′-TAMRA | | | | |
| Reverse | 5′-CCCTCGTCCTGTCTTACCCT-3′ | 59.7 | 20 | 230 | 47 |

TABLE 25
Panel 1.3D

| | Relative |
|---|---|
| | Expression (%) |
| | 1.3dx4tm5382f |
| Tissue Name | _ag3076_b1 |
| Liver adenocarcinoma | 0.0 |
| Pancreas | 0.0 |
| Pancreatic ca. CAPAN 2 | 0.0 |
| Adrenal gland | 0.0 |
| Thyroid | 0.0 |
| Salivary gland | 0.0 |
| Pituitary gland | 0.0 |
| Brain (fetal) | 0.0 |
| Brain (whole) | 0.0 |
| Brain (amygdala) | 0.0 |
| Brain (cerebellum) | 0.0 |
| Brain (hippocampus) | 0.0 |
| Brain (substantia nigra) | 0.0 |
| Brain (thalamus) | 0.0 |
| Cerebral Cortex | 0.0 |
| Spinal cord | 0.0 |
| CNS ca. (glio/astro) U87-MG | 0.0 |
| CNS ca. (glio/astro) U-118-MG | 0.0 |
| CNS ca. (astro) SW1783 | 0.0 |
| CNS ca.* (neuro; met) SK-N-AS | 0.0 |
| CNS ca. (astro) SF-539 | 0.0 |
| CNS ca. (astro) SNB-75 | 0.0 |
| CNS ca. (glio) SNB-19 | 0.0 |
| CNS ca. (glio) U251 | 0.0 |
| CNS ca. (glio) SF-295 | 0.0 |
| Heart (fetal) | 0.0 |
| Heart | 0.0 |
| Fetal Skeletal | 0.0 |
| Skeletal muscle | 0.0 |
| Bone marrow | 0.4 |
| Thymus | 0.0 |
| Spleen | 0.0 |
| Lymph node | 0.2 |
| Colorectal | 0.2 |
| Stomach | 0.0 |
| Small intestine | 0.0 |
| Colon ca. SW480 | 0.0 |
| Colon ca.* (SW480 met)SW620 | 0.0 |
| Colon ca. HT29 | 0.0 |
| Colon ca. HCT-116 | 0.0 |
| Colon ca. CaCo-2 | 0.0 |
| 83219 CC Well to Mod Diff (OD03866) | 0.0 |
| Colon ca. HCC-2998 | 0.0 |
| Gastric ca.* (liver met) NCI-N87 | 0.0 |
| Bladder | 0.0 |
| Trachea | 0.0 |
| Kidney | 0.0 |
| Kidney (fetal) | 0.0 |
| Renal ca. 786-0 | 0.0 |
| Renal ca. A498 | 0.0 |
| Renal ca. RXF 393 | 0.0 |
| Renal ca. ACHN | 0.0 |
| Renal ca. UO-31 | 0.0 |
| Renal ca. TK-10 | 0.0 |
| Liver | 0.0 |
| Liver (fetal) | 0.0 |
| Liver ca. (hepatoblast) HepG2 | 0.0 |
| Lung | 0.0 |
| Lung (fetal) | 0.0 |
| Lung ca. (small cell) LX-1 | 0.0 |
| Lung ca. (small cell) NCI-H69 | 0.0 |
| Lung ca. (s.cell var.) SHP-77 | 0.0 |
| Lung ca. (large cell)NCI-H460 | 0.0 |
| Lung ca. (non-sm. cell) A549 | 0.0 |
| Lung ca. (non-s.cell) NCI-H23 | 0.2 |
| Lung ca (non-s.cell) HOP-62 | 0.0 |
| Lung ca. (non-s.cl) NCI-H522 | 0.0 |
| Lung ca. (squam.) SW 900 | 0.0 |
| Lung ca. (squam.) NCI-H596 | 0.0 |
| Mammary gland | 0.0 |
| Breast ca.* (p1. effusion) MCF-7 | 0.0 |
| Breast ca.* (p1.ef) MDA-MB-231 | 0.0 |
| Breast ca.* (p1. effusion) T47D | 0.0 |
| Breast ca. BT-549 | 0.0 |
| Breast ca. MDA-N | 0.0 |
| Ovary | 0.0 |
| Ovarian ca. OVCAR-3 | 0.0 |
| Ovarian ca. OVCAR-4 | 0.0 |
| Ovarian ca. OVCAR-5 | 0.2 |
| Ovarian ca. OVCAR-8 | 0.0 |
| Ovarian ca. IGROV-1 | 0.0 |
| Ovarian ca.* (ascites) SK-OV-3 | 0.0 |
| Uterus | 0.0 |
| Placenta | 0.0 |
| Prostate | 0.0 |
| Prostate ca.* (bone met)PC-3 | 0.0 |
| Testis | 0.0 |
| Melanoma Hs688(A).T | 0.0 |
| Melanoma* (met) Hs688(B).T | 0.0 |
| Melanoma UACC-62 | 0.0 |
| Melanoma M14 | 100.0 |
| Melanoma LOX IMVI | 0.0 |
| Melanoma* (met) SK-MEL-5 | 0.0 |
| Adipose | 0.0 |

Panel 1.3D Summary: Ag3076 The AC010784-1 gene encodes a protein with homology to oncostatin. This gene is exclusively expressed in a sample derived from melanoma cell line M14 (CT=27). Thus, the AC010784-1 gene could be used to distinguish this sample from other samples and may play a role in melanoma.

Panel 2.2 Summary: Ag3076 Expression of the AC010784-1 gene is low/undetectable (CTs>34.3) across the samples on this panel (data not shown).

Panel 4D Summary: Ag3076 Expression of the AC010784-1 gene is low/undetectable (CTs>35) across the samples on this panel (data not shown).

G. Clone Identification No: AL049825/CG54029-01 (PTMA 8)

Expression of gene AL049825 was assessed using the primer-probe set Ag2649, described in Table 26. Results from RTQ-PCR run are shown in Table 27, 28, and 29.

TABLE 26
Probe Name Ag2649

| | | | | Start | SEQ ID |
|---|---|---|---|---|---|
| Primers | Sequences | TM | Length | Position | NO: |
| Forward | 5′-AGCGTAATGTCCATGTTGTTCT-3′ | 58.7 | 22 | 7 | 48 |
| Probe | FAM-5′- | 68.8 | 26 | 39 | 49 |
| | CACAGCTTTTCTGATCGGCATACAGG- | | | | |
| | 3′-TAMRA | | | | |
| Reverse | 5′-GGGAGATTGCTCTCTGAGTGT-3′ | 59.3 | 21 | 72 | 50 |

TABLE 27
Panel 1.3D

| | Relative |
|---|---|
| | Expression (%) |
| | 1.3dtm4673f_{—} |
| Tissue Name | ag2649 |
| Liver adenocarcinoma | 0.0 |
| Pancreas | 0.4 |
| Pancreatic ca. CAPAN 2 | 0.0 |
| Adrenal gland | 1.7 |
| Thyroid | 0.3 |
| Salivary gland | 1.3 |
| Pituitary gland | 1.8 |
| Brain (fetal) | 0.0 |
| Brain (whole) | 1.2 |
| Brain (amygdala) | 1.2 |
| Brain (cerebellum) | 0.7 |
| Brain (hippocampus) | 3.4 |
| Brain (substantia nigra) | 0.3 |
| Brain (thalamus) | 0.6 |
| Cerebral Cortex | 13.4 |
| Spinal cord | 3.6 |
| CNS ca. (glio/astro) U87-MG | 33.0 |
| CNS ca. (glio/astro) U-118-MG | 2.0 |
| CNS ca. (astro) SW1783 | 7.7 |
| CNS ca.* (neuro; met) SK-N-AS | 0.6 |
| CNS ca. (astro) SF-539 | 0.0 |
| CNS ca. (astro) SNB-75 | 4.9 |
| CNS ca. (glio) SNB-19 | 0.4 |
| CNS ca. (glio) U251 | 0.9 |
| CNS ca. (glio) SF-295 | 0.1 |
| Heart (fetal) | 51.8 |
| Heart | 14.1 |
| Fetal Skeletal | 51.0 |
| Skeletal muscle | 3.2 |
| Bone marrow | 0.0 |
| Thymus | 3.5 |
| Spleen | 1.8 |
| Lymph node | 1.0 |
| Colorectal | 4.0 |
| Stomach | 1.5 |
| Small intestine | 3.9 |
| Colon ca. SW480 | 0.0 |
| Colon ca.* (SW480 met)SW620 | 0.0 |
| Colon ca. HT29 | 0.0 |
| Colon ca. HCT-116 | 0.0 |
| Colon ca. CaCo-2 | 0.0 |
| 83219 CC Well to Mod Diff (OD03866) | 4.1 |
| Colon Ca. HCC-2998 | 0.0 |
| Gastric ca.* (liver met) NCI-N87 | 0.0 |
| Bladder | 1.2 |
| Trachea | 3.3 |
| Kidney | 2.3 |
| Kidney (fetal) | 2.7 |
| Renal ca. 786-0 | 5.2 |
| Renal ca. A498 | 1.3 |
| Renal ca. RXF 393 | 14.8 |
| Renal ca. ACHN | 2.0 |
| Renal ca. UO-31 | 1.0 |
| Renal ca. TK-10 | 0.0 |
| Liver | 2.3 |
| Liver (fetal) | 0.7 |
| Liver ca. (hepatoblast) HepG2 | 0.0 |
| Lung | 0.0 |
| Lung (fetal) | 1.6 |
| Lung ca. (small cell) LX-1 | 0.0 |
| Lung ca. (small cell) NCI-H69 | 0.0 |
| Lung ca. (s.cell var.) SHP-77 | 9.2 |
| Lung ca. (large cell)NCI-H460 | 0.0 |
| Lung ca. (non-sm. cell) A549 | 0.0 |
| Lung ca. (non-s.cell) NCI-H23 | 0.3 |
| Lung ca (non-s.cell) HOP-62 | 0.0 |
| Lung ca. (non-s.cl) NCI-H522 | 0.0 |
| Lung ca. (squam.) SW 900 | 9.9 |
| Lung ca. (squam.) NCI-H596 | 0.0 |
| Mammary gland | 3.9 |
| Breast ca.* (p1. effusion) MCF-7 | 0.0 |
| Breast ca.* (p1.ef) MDA-MB-231 | 13.2 |
| Breast ca.* (p1. effusion) T47D | 0.0 |
| Breast ca. BT-549 | 14.9 |
| Breast ca. MDA-N | 0.0 |
| Ovary | 100.0 |
| Ovarian ca. OVCAR-3 | 0.3 |
| Ovarian ca. OVCAR-4 | 0.0 |
| Ovarian ca. OVCAR-5 | 0.0 |
| Ovarian ca. OVCAR-8 | 35.4 |
| Ovarian ca. IGROV-1 | 0.0 |
| Ovarian ca.* (ascites) SK-OV-3 | 7.9 |
| Uterus | 3.8 |
| Placenta | 1.9 |
| Prostate | 1.0 |
| Prostate ca.* (bone met)PC-3 | 1.8 |
| Testis | 2.6 |
| Melanoma Hs688(A).T | 29.7 |
| Melanoma* (met) Hs688(B).T | 29.1 |
| Melanoma UACC-62 | 0.3 |
| Melanoma M14 | 0.0 |
| Melanoma LOX IMVI | 0.0 |
| Melanoma* (met) SK-MEL-5 | 0.3 |
| Adipose | 14.4 |

TABLE 28
Panel 2D

| | Relative | | Relative |
|---|---|---|---|
| | Expression(%) | | Expression(%) |
| | 2dx4tm4662f_{—} | | 2dx4tm4662f_{—} |
| Tissue Name | ag2649_a1 | Tissue Name | ag2649_a1 |
| Normal Colon GENPAK | 46.9 | Kidney NAT Clontech 8120608 | 12.0 |
| 061003 | | | |
| 83219 CC Well to Mod Diff | 12.7 | Kidney Cancer Clontech | 5.0 |
| (ODO3866) | | 8120613 | |
| 83220 CC NAT (ODO3866) | 10.5 | Kidney NAT Clontech 8120614 | 26.6 |
| 83221 CC Gr.2 rectosigmoid | 1.9 | Kidney Cancer Clontech | 28.8 |
| (ODO3868) | | 9010320 | |
| 83222 CC NAT (ODO3868) | 0.6 | Kidney NAT Clontech 9010321 | 28.8 |
| 83235 CC Mod Diff | 1.0 | Normal Uterus GENPAK | 37.9 |
| (ODO3920) | | 061018 | |
| 83236 CCNAT (ODO3920) | 18.7 | Uterus Cancer GENPAK | 82.1 |
| | | 064011 | |
| 83237 CC Gr.2 ascend colon | 3.4 | Normal Thyroid Clontech A+ | 5.2 |
| (ODO3921) | | 6570-1 | |
| 83238 CC NAT (ODO3921) | 9.2 | Thyroid Cancer GENPAK | 19.5 |
| | | 064010 | |
| 83241 CC from Partial | 7.0 | Thyroid Cancer INVITROGEN | 6.8 |
| Hepatectomy (ODO4309) | | A302152 | |
| 83242 Liver NAT (ODO4309) | 13.7 | Thyroid NAT INVITROGEN | 18.5 |
| | | A302153 | |
| 87472 Colon mets to lung | 0.0 | Normal Breast GENPAK | 24.1 |
| (OD04451-01) | | 061019 | |
| 87473 Lung NAT (OD04451- | 5.8 | 84877 Breast Cancer | 7.8 |
| 02) | | (OD04566) | |
| Normal Prostate Clontech A+ | 26.7 | 85975 Breast Cancer | 17.2 |
| 6546-1 | | (OD04590-01) | |
| 84140 Prostate Cancer | 6.6 | 85976 Breast Cancer Mets | 22.1 |
| (OD04410) | | (OD04590-03) | |
| 84141 Prostate NAT | 23.2 | 87070 Breast Cancer Metastasis | 1.1 |
| (OD04410) | | (OD04655-05) | |
| 87073 Prostate Cancer | 8.2 | GENPAK Breast Cancer | 3.4 |
| (OD04720-01) | | 064006 | |
| 87074 Prostate NAT | 19.1 | Breast Cancer Res. Gen. 1024 | 17.0 |
| (OD04720-02) | | | |
| Normal Lung GENPAK 061010 | 27.3 | Breast Cancer Clontech | 9.2 |
| | | 9100266 | |
| 83239 Lung Met to Muscle | 6.5 | Breast NAT Clontech 9100265 | 13.2 |
| (ODO4286) | | | |
| 83240 Muscle NAT | 15.1 | Breast Cancer INVITROGEN | 25.5 |
| (ODO4286) | | A209073 | |
| 84136 Lung Malignant Cancer | 7.2 | Breast NAT INVITROGEN | 29.7 |
| (OD03126) | | A2090734 | |
| 84137 Lung NAT (OD03126) | 14.7 | Normal Liver GENPAK | 14.5 |
| | | 061009 | |
| 84871 Lung Cancer (OD04404) | 30.2 | Liver Cancer GENPAK 064003 | 0.5 |
| 84872 Lung NAT (OD04404) | 30.4 | Liver Cancer Research | 12.3 |
| | | Genetics RNA 1025 | |
| 84875 Lung Cancer (OD04565) | 8.9 | Liver Cancer Research | 17.3 |
| | | Genetics RNA 1026 | |
| 84876 Lung NAT (OD04565) | 15.0 | Paired Liver Cancer Tissue | 21.3 |
| | | Research Genetics RNA 6004- | |
| | | T | |
| 85950 Lung Cancer (OD04237- | 1.2 | Paired Liver Tissue Research | 4.8 |
| 01) | | Genetics RNA 6004-N | |
| 85970 Lung NAT (OD04237- | 13.8 | Paired Liver Cancer Tissue | 10.1 |
| 02) | | Research Genetics RNA 6005- | |
| | | T | |
| 83255 Ocular Mel Met to Liver | 2.3 | Paired Liver Tissue Research | 10.0 |
| (ODO4310) | | Genetics RNA 6005-N | |
| 83256 Liver NAT (ODO4310) | 26.6 | Normal Bladder GENPAK | 12.9 |
| | | 061001 | |
| 84139 Melanoma Mets to Lung | 0.9 | Bladder Cancer Research | 2.4 |
| (OD04321) | | Genetics RNA 1023 | |
| 84138 Lung NAT (OD04321) | 5.8 | Bladder Cancer INVITROGEN | 6.0 |
| | | A302173 | |
| Normal Kidney GENPAK | 39.5 | 87071 Bladder Cancer | 11.9 |
| 061008 | | (OD04718-01) | |
| 83786 Kidney Ca, Nuclear | 15.4 | 87072 Bladder Normal | 69.9 |
| grade 2 (OD04338) | | Adjacent (OD04718-03) | |
| 83787 Kidney NAT (OD04338) | 35.9 | Normal Ovary Res. Gen. | 100.0 |
| 83788 Kidney Ca Nuclear grade | 3.7 | Ovarian Cancer GENPAK | 44.3 |
| 1/2 (OD04339) | | 064008 | |
| 83789 Kidney NAT (OD04339) | 33.3 | 87492 Ovary Cancer | 6.2 |
| | | (OD04768-07) | |
| 83790 Kidney Ca, Clear cell | 99.9 | 87493 Ovary NAT (OD04768- | 89.3 |
| type (OD04340) | | 08) | |
| 83791 Kidney NAT (OD04340) | 21.4 | Normal Stomach GENPAK | 18.8 |
| | | 061017 | |
| 83792 Kidney Ca, Nuclear | 8.2 | Gastric Cancer Clontech | 5.3 |
| grade 3 (OD04348) | | 9060358 | |
| 83793 Kidney NAT (OD04348) | 18.4 | NAT Stomach Clontech | 6.0 |
| | | 9060359 | |
| 87474 Kidney Cancer | 22.9 | Gastric Cancer Clontech | 9.9 |
| (OD04622-01) | | 9060395 | |
| 87475 Kidney NAT (OD04622- | 10.2 | NAT Stomach Clontech | 5.4 |
| 03) | | 9060394 | |
| 85973 Kidney Cancer | 1.8 | Gastric Cancer Clontech | 12.9 |
| (OD04450-01) | | 9060397 | |
| 85974 Kidney NAT (OD04450- | 39.4 | NAT Stomach Clontech | 3.9 |
| 03) | | 9060396 | |
| Kidney Cancer Clontech | 15.5 | Gastric Cancer GENPAK | 12.8 |
| 8120607 | | 064005 | |

TABLE 29
Panel 4D

| | Relative | | Relative |
|---|---|---|---|
| | Expression(%) | | Epression(%) |
| | 4dx4tm4179f_{—} | | 4dx4tm4179f_{—} |
| Tissue Name | ag2649_a1 | Tissue Name | ag2649_a1 |
| 93768_Secondary Th1_anti- | 0.0 | 93100_HUVEC | 0.0 |
| CD28/anti-CD3 | | (Endothelial)_IL-1b | |
| 93769_Secondary Th2_anti- | 0.0 | 93779_HUVEC | 0.0 |
| CD28/anti-CD3 | | (Endothelial)_IFN gamma | |
| 93770_Secondary Tr1_anti- | 0.0 | 93102_HUVEC | 0.0 |
| CD28/anti-CD3 | | (Endothelial)_TNF alpha + IFN | |
| | | gamma | |
| 93573_Secondary Th1_resting | 0.0 | 93101_HUVEC | 0.0 |
| day 4-6 in IL-2 | | (Endothelial)_TNF alpha + IL4 | |
| 93572_Secondary Th2_resting | 0.0 | 93781_HUVEC | 0.0 |
| day 4-6 in IL-2 | | (Endothelial)_IL-11 | |
| 93571_Secondary Tr1_resting | 0.0 | 93583_Lung Microvascular | 0.4 |
| day 4-6 in IL-2 | | Endothelial Cells_none | |
| 93568_primary Th1_anti- | 0.0 | 93584_Lung Microvascular | 6.3 |
| CD28/anti-CD3 | | Endothelial Cells_TNFa (4 | |
| | | ng/ml) and IL 1b (1 ng/ml) | |
| 93569_primary Th2_anti- | 0.0 | 92662_Microvascular Dermal | 0.3 |
| CD28/anti-CD3 | | endothelium_none | |
| 93570_primary Tr1_anti- | 0.0 | 92663_Microsvasular Dermal | 3.7 |
| CD28/anti-CD3 | | endothelium_TNFa (4 ng/ml) | |
| | | and IL 1b (1 ng/ml) | |
| 93565_primary Th1_resting dy | 0.0 | 93773_Bronchial | 0.0 |
| 4-6 in IL-2 | | epithelium_TNFa (4 ng/ml) and | |
| | | IL 1b (1 ng/ml)** | |
| 93566_primary Th2_resting dy | 0.0 | 93347_Small Airway | 0.7 |
| 4-6 in IL-2 | | Epithelium_none | |
| 93567_primary Tr1_resting dy | 0.0 | 93348_Small Airway | 2.5 |
| 4-6 in IL-2 | | Epithelium_INFa (4 ng/ml) | |
| | | and IL 1b (1 ng/ml) | |
| 93351_CD45RA CD4 | 21.0 | 92668_Coronery Artery | 9.3 |
| lymphocyte_anti-CD28/anti- | | SMC_resting | |
| CD3 | | | |
| 93352_CD45RO CD4 | 0.0 | 92669_Coronery Artery | 9.0 |
| lymphocyte_anti-CD28/anti- | | SMC_TNFa (4 ng/ml) and IL 1b | |
| CD3 | | (1 ng/ml) | |
| 93251_CD8 Lymphocytes_anti- | 0.0 | 93107_astrocytes_resting | 21.1 |
| CD28/anti-CD3 | | | |
| 93353_chronic CD8 | 0.0 | 93108_astrocytes_TNFa (4 | 65.2 |
| Lymphocytes 2ry_resting dy 4- | | ng/ml) and IL 1b (1 ng/ml) | |
| 6 in IL-2 | | | |
| 93574_chronic CD8 | 0.0 | 92666_KU-812 | 0.0 |
| Lymphocytes 2ry_activated | | (Basophil)_resting | |
| CD3/CD28 | | | |
| 93354_CD4_none | 0.0 | 92667_KU-812 | 0.0 |
| | | (Basophil)_PMA/ionoycin | |
| 93252_Secondary | 0.0 | 93579_CCD1106 | 3.4 |
| Th1/Th2/Trl_anti-CD95 CH11 | | (Keratinocytes)_none | |
| 93103_LAX cells_resting | 0.0 | 93580_CCD1106 | 0.1 |
| | | (Keratinocytes)_TNFa and | |
| | | IFNg** | |
| 93788_LAX cells_IL-2 | 0.0 | 93791_Liver Cirrhosis | 1.7 |
| 93787_LAX cells_IL-2 + IL-12 | 0.0 | 93792_Lupus Kidney | 0.4 |
| 93789_LAX cells_IL-2 + IFN | 0.0 | 93577_NCI-H292 | 0.0 |
| gamma | | | |
| 93790_LAX cells_IL-2 + IL-18 | 0.0 | 93358_NCI-H292_IL-4 | 0.0 |
| 93104_LAK | 0.0 | 93360_NCI-H292_IL-9 | 0.0 |
| cells_PMA/ionomycin and IL- | | | |
| 18 | | | |
| 93578_NK Cells IL-2_resting | 0.0 | 93359_NCI-H292_IL-13 | 0.0 |
| 93109_Mixed Lymphocyte | 0.0 | 93357_NCI-H292_IFN gamma | 0.0 |
| Reaction_Two Way MLR | | | |
| 93110_Mixed Lymphocyte | 0.0 | 93777_HPAEC_- | 0.0 |
| Reaction_Two Way MLR | | | |
| 93111_Mixed Lymphocyte | 0.0 | 93778_HPAEC_IL-1 beta/TNA | 0.0 |
| Reaction_Two Way MLR | | alpha | |
| 93112_Mononuclear Cells | 0.0 | 93254_Normal Human Lung | 2.7 |
| (PBMCs)_resting | | Fibroblast_none | |
| 93113_Mononuclear Cells | 0.0 | 93253_Normal Human Lung | 2.9 |
| (PBMCs)_PWM | | Fibroblast_TNFa (4 ng/ml) and | |
| | | IL-1b (1 ng/ml) | |
| 93114_Mononuclear Cells | 0.0 | 93257_Normal Human Lung | 3.0 |
| (PBMCs)_PHA-L | | Fibroblast_IL-4 | |
| 93249_Ramos (B cell)_none | 0.0 | 93256_Normal Human Lung | 7.4 |
| | | Fibroblast_IL-9 | |
| 93250_Ramos (B | 0.0 | 93255_Normal Human Lung | 1.5 |
| cell)_ionomycin | | Fibroblast_IL-13 | |
| 93349_B lymphocytes_PWM | 0.0 | 93258_Normal Human Lung | 4.9 |
| | | Fibroblast_IFN gamma | |
| 93350_B lymphoytes_CD40L | 0.0 | 93106_Dermal Fibroblasts | 88.7 |
| and IL-4 | | CCD1070_resting | |
| 92665_EOL-1 | 0.0 | 93361_Dermal Fibroblasts | 100.0 |
| (Eosinophil)_dbcAMP | | CCD1070_TNF alpha 4 ng/ml | |
| differentiated | | | |
| 93248_EOL-1 | 0.0 | 93105_Dermal Fibroblasts | 93.4 |
| (Eosinophil)_dbcAMP/PMAion | | CCD1070_IL-1 beta 1 ng/ml | |
| omycin | | | |
| 93356_Dendritic Cells_none | 0.0 | 93772_dermal fibroblast_IFN | 20.8 |
| | | gamma | |
| 93355_Dendritic Cells_LPS | 0.0 | 93771_dermal fibroblast_IL-4 | 10.8 |
| 100 ng/ml | | | |
| 93775_Dendritic Cells_anti- | 0.0 | 93260_IBD Colitis 2 | 0.0 |
| CD40 | | | |
| 93774_Monocytes_resting | 0.0 | 93261_IBD Crohns | 0.5 |
| 93776_Monocytes_LPS 50 | 0.1 | 735010_Colon_normal | 0.8 |
| ng/ml | | | |
| 93581_Macrophages_resting | 0.0 | 735019_Lung_none | 2.4 |
| 93582_Macrophages_LPS 100 | 0.0 | 64028-1_Thymus_none | 1.9 |
| ng/ml | | | |
| 93098_HUVEC | 0.0 | 64030-1_Kidney_none | 0.4 |
| (Endothelial)_none | | | |
| 93099_HUVEC | 0.0 | | |
| (Endothelial)_starved | | | |

Panel 1.3D Summary: Ag2649 The AL049825 gene encodes the beta polypeptide of nerve growth factor (NGF) and is known publicly. Expression of the AL049825 gene is highest in a sample derived from normal ovarian tissue (CT=28.4). Consistent with this observation, nerve growth factor is required for early follicular development in the mammalian ovary (ref. 1). Thus, the expression of this gene could be used to distinguish normal ovary tissue from other tissues and modulation of AL049825 gene product activity could potentially be used to treat diseases of the ovary. Furthermore, there is also substantial expression in cancer cell lines derived from two melanomas, an ovarian cancer and a brain cancer.

Among CNS tissues, the AL049825 gene is expressed at low but significant levels in cerebral cortex, spinal cord, and hippocampus (CTs=31-33). NGF has been shown to have neuroprotective effects in cholinergic neurons in response to axotomy and actually stimulates survival and regeneration (ref. 2). A deficit in cholinergic neurons is a hallmark of Alzheimer's disease, and treatment with acetylcholine esterase inhibitors is a standard of care (ref. 3). Thus, therapeutic modulation of the AL049825 gene or treatment with its protein product may be of use in treating Alzheimer's disease, or any other condition characterized by neuronal loss/degeneration, including Huntington's disease, Parkinson's disease, stroke, head trauma, spinal cord injury or spinocerebellar ataxia.

Among metabolic tissues, the AL049825 gene is expressed at low levels in adrenal gland and pituitary gland (CT=34) and at moderate levels in adipose and heart (CTs=29-31). Thus, this gene product may be a potential protein therapeutic for the treatment of disease in these tissues. Interestingly, this gene also appears to be more highly expressed in fetal skeletal muscle tissue (CT=29) when compared to adult skeletal muscle tissue (CT=33). Therefore, expression of the AL049825 gene could be used to distinguish fetal skeletal muscle from adult skeletal muscle. Moreover, therapeutic modulation of this gene product, through the use of small molecule drugs, antibodies or the protein itself, could be used to treat muscle degenerative diseases.

Panel 2D Summary: Ag2649 Expression of the AL049825 gene is highest in a sample derived from normal ovarian tissue (CT=29.2); this result is consistent with what is observed in Panel 1.3D. Interestingly, expression is down regulated in two ovarian tumors relative to the matched normal margins. There also appears to be substantial expression in a number of other normal tissues adjacent to cancers. These tissues include prostate, liver, kidney and bladder. Thus, expression of the AL049825 gene could be used distinguish as a marker to distinguish between normal and cancerous tissue in the above listed organ sites. Moreover, therapeutic modulation of the expression or function of this gene product, through the use of small molecule drugs, antibodies or protein therapeutics, might be of benefit in the treatment of prostate, ovarian, liver, kidney or bladder cancer.

Panel 4D Summary: Ag2649 The AL049825 gene is highly expressed in dermal fibroblasts independently of their activation status (untreated or treated with cytokines TNF□ or IL-1□). It is also up regulated (3 fold) in astrocytes treated with TNF-□ and IL-1□. The expression of this gene in dermal fibroblast might result from rich inervations of this tissue. The protein encoded by the AL049825 gene belongs to the nerve growth factor family, which is important for the development and maintenance of nerve cells. The presence of this transcript in astrocytes may suggest an attempt from the astrocytes to compensate initial damage to inflammatory reactions occurring in the CNS. Thus, therapeutic modulation of the AL049825 protein using either antibodies or protein therapeutics could be used to treat CNS diseases associated with inflammatory reactions, including multiple sclerosis, stroke, or neurodegeneration. In addition, this protein might play a role in immune related disease such as autoimmune diseases. (Dissen et al., Nerve growth factor is required for early follicular development in the mammalian ovary. Endocrinology 142: 2078-2086, 2001; Eagle et al., Axonal regeneration and limited functional recovery following hippocampal deafferentation. J. Comp. Neurol. 363: 377-388, 1995; Fahnestock et al., The precursor pro-nerve growth factor is the predominant form of nerve growth factor in brain and is increased in Alzheimer's disease. Mol. Cell. Neurosci. 18:210-220, 2001.)

### Example 3

### Novel Single Nucleotide Polymorphisms (SNPs) for AL121585_da3

SNP variants were obtained from assemblies as follows: cDNA was derived from various human samples representing multiple tissue types, normal and diseased states, physiological states, and developmental states from different donors. Samples were obtained as whole tissue, cell lines, primary cells or tissue cultured primary cells and cell lines. Cells and cell lines may have been treated with biological or chemical agents that regulate gene expression for example, growth factors, chemokines, steroids. The cDNA thus derived was then sequenced using CuraGen Corporation's SeqCalling technology disclosed in copending application U.S. Ser. No. 09/417,386 filed Oct. 13, 1999, incorporated herein in its entirety. Sequence traces were evaluated manually and edited for corrections if appropriate. cDNA sequences from all samples were assembled with themselves and with public ESTs using bioinformatics programs to generate CuraGen's human SeqCalling database of SeqCalling assemblies. Each assembly contains one or more overlapping cDNA sequences derived from one or more human samples. Fragments and ESTs were included as components for an assembly when the extent of identity with another component of the assembly was at least 95% over 50 bp. Each assembly can represent a gene and/or its variants such as splice forms and/or single nucleotide polymorphisms (SNPs) and their combinations.

A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent where a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern for example, alteration in temporal expression, physiological response regulation, cell type expression regulation, intensity of expression, or stability of transcribed message.

Method of novel SNP Identification: SNPs are identified by analyzing sequence assemblies using CuraGen's proprietary SNPTool algorithm. SNPTool identifies variation in assemblies with the following criteria: SNPs are not analyzed within 10 base pairs on both ends of an alignment; window size (number of bases in a view) is 10; allowed number of mismatches in a window is 2; minimum SNP base quality (PHRED score) is 23; minimum number of changes to score an SNP is 2/assembly position. SNPTool analyzes the assembly and displays SNP positions, associated individual variant sequences in the assembly, the depth of the assembly at that given position, the putative assembly allele frequency, and the SNP sequence variation. Sequence traces are then selected and brought into view for manual validation. The consensus assembly sequence is imported into CuraTools along with variant sequence changes to identify potential amino acid changes resulting from the SNP sequence variation. Comprehensive SNP data analysis is then exported into the SNPCalling database.

The SNP variants found in AL121585_da3 (SEQ ID NO.21) are shown in Table 4. Variants reported in Table 4 for AL121585_da3 may occur in any nucleic acid sequence either individually or in any combination of more than one.

TABLE 30

| | | | | | Major | Minor |
|---|---|---|---|---|---|---|
| Con- | | | Major | Minor | Allele | Allele |
| sensus | | Allele | Allele | Allele | Amino | Amino |
| Position | Depth | Frequency | Nucleotide | Nucleotide | Acid | Acid |
| 202 | 20 | 0.45 | T | C | Val | Cys |
| 209 | 20 | 0.45 | A | G | Glu | Arg |
| 212 | 20 | 0.45 | G | A | Ala | Thr |
| 215 | 20 | 0.45 | A | Glu | Arg | |

A nucleotide sequence and an amino acid sequence that incorporate the SNPs shown in Table 4 are disclosed in SEQ ID NOS:51 and 52 respectively.

AL121585_da3 incorporating the SNPs has the following sequence:

attgttcctt gtccggctcc ttgctcgccg cagccgcctt taccgctgcg gactccggac acttcatcac cacagtccct gaactctcgc tttcttttta atcccctgca tcggatcact ggtktgccgg acc atg tca gac gca gcc gta gac acc agc tcc gaa atcacc acc aag gac tta aag gag aag aag gaa gty gtg gaa rag rca raa aat gga aga gac gcc cct gct aac ggg aat gct aat gag gaa aat ggg gag cag gag gct gac aat gag gta gac gaa gaa gag gaa gaa ggt ggg gag gaa gag gag gag gaa gaa gaa ggt gat ggt gag gaa gag gat gga gat gaa gat gag gaa gct gag tca gct acg ggc aag cgg gca gct gaa gat gat gag gat gac gat gtc gat acc aag aag cag aag acc aac aag gat gac tagacagcaa aaaaggaaat gttaggaggg tgac (SEQ ID NO:51)

The polypeptide encoded by clone AL121585_da3 including the incorporated SNPs has the following sequence:

Met Ser Asp Ala Ala Val Asp Thr Ser Ser Glu Ile Thr Thr Lys Asp Leu Lys Glu Lys Lys Glu Xaa Val Glu Xaa Xaa Xaa Asn Gly Arg Asp Ala Pro Ala Asn Gly Asn Ala Asn Glu Glu Asn Gly Glu Gln Glu Ala Asp Asn Glu Val Asp Glu Glu Glu Glu Glu Gly Gly Glu Glu Glu Glu Glu Glu Glu Glu Gly Asp Gly Glu Glu Glu Asp Gly Asp Glu Asp Glu Glu Ala Glu Ser Ala Thr Gly Lys Arg Ala Ala Glu Asp Asp Glu Asp Asp Asp Val Asp Thr Lys Lys Gln Lys Thr Asn Lys Asp Asp (SEQ ID NO:52)

### Other Embodiment

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims

What is claimed is:

1. An isolated polypeptide comprising an amino acid sequence selected from the group consisting of:
a) a mature form of the amino acid sequence given by SEQ ID NO:2, 4, 6, 8, 10, 12, 14, 16, 18, 20, 22, 24, 26 and 52;
b) a variant of a mature form of the amino acid sequence given by SEQ ID NO:2, 4, 6, 8, 10, 12, 14, 16, 18, 20, 22, 24, 26 and 52, wherein any amino acid in the mature form is changed to a different amino acid, provided that no more than 15% of the amino acid residues in the sequence of the mature form are so changed;
c) the amino acid sequence given by SEQ ID NO:2, 4, 6, 8, 10, 12, 14, 16, 18, 20, 22, 24, 26 and 52;
d) a variant of the amino acid sequence given by SEQ ID NO:2, 4, 6, 8, 10, 12, 14, 16, 18, 20, 22, 24, 26 and 52 wherein any amino acid specified in the chosen sequence is changed to a different amino acid, provided that no more than 15% of the amino acid residues in the sequence are so changed; and
e) a fragment of any of a) through d).

2. The polypeptide of claim 1 that is a naturally occurring allelic variant of the sequence given by SEQ ID NO:2, 4, 6, 8, 10, 12, 14, 16, 18, 20, 22, 24, 26 and 52.

3. The polypeptide of claim 2, wherein the variant is the translation of a single nucleotide polymorphism.

4. The polypeptide of claim 1 that is a variant polypeptide described therein, wherein any amino acid specified in the chosen sequence is changed to provide a conservative substitution.

5. An isolated nucleic acid molecule comprising a nucleic acid sequence encoding a polypeptide comprising an amino acid sequence selected from the group consisting of:
a) a mature form of the amino acid sequence given SEQ ID NO:2, 4, 6, 8, 10, 12, 14, 16, 18, 20, 22, 24, 26 and 52;
b) a variant of a mature form of the amino acid sequence given by SEQ ID NO:2, 4, 6, 8, 10, 12, 14, 16, 18, 20, 22, 24, 26 and 52 wherein any amino acid in the mature form of the chosen sequence is changed to a different amino acid, provided that no more than 15% of the amino acid residues in the sequence of the mature form are so changed;
c) the amino acid sequence given by SEQ ID NO:2, 4, 6, 8, 10, 12, 14, 16, 18, 20, 22, 24,26 and 52;
d) a variant of the amino acid sequence given by SEQ ID NO:2, 4, 6, 8, 10, 12, 14, 16, 18, 20, 22, 24, 26 and 52, in which any amino acid specified in the chosen sequence is changed to a different amino acid, provided that no more than 15% of the amino acid residues in the sequence are so changed;
e) a nucleic acid fragment encoding at least a portion of a polypeptide comprising the amino acid sequence given by SEQ ID NO:2, 4, 6, 8, 10, 12, 14, 16, 18, 20, 22, 24, 26 and 52 or any variant of said polypeptide wherein any amino acid of the chosen sequence is changed to a different amino acid, provided that no more than 10% of the amino acid residues in the sequence are so changed; and
f) the complement of any of said nucleic acid molecules.

6. The nucleic acid molecule of claim 5, wherein the nucleic acid molecule comprises the nucleotide sequence of a naturally occurring allelic nucleic acid variant.

7. The nucleic acid molecule of claim 5 that encodes a variant polypeptide, wherein the variant polypeptide has the polypeptide sequence of a naturally occurring polypeptide variant.

8. The nucleic acid molecule of claim 5, wherein the nucleic acid molecule comprises a single nucleotide polymorphism encoding said variant polypeptide.

9. The nucleic acid molecule of claim 5, wherein said nucleic acid molecule comprises a nucleotide sequence selected from the group consisting of
a) the nucleotide sequence given by SEQ ID NO:1, 3, 5, 7, 9, 11, 13, 15, 17, 19, 21, 23, 25 and 51;
b) a nucleotide sequence wherein one or more nucleotides in the nucleotide sequence given by SEQ ID NO:1, 3, 5, 7, 9, 11, 13, 15, 17, 19, 21, 23, 25 and 51 is changed from that given by the chosen sequence to a different nucleotide provided that no more than 15% of the nucleotides are so changed;
c) a nucleic acid fragment of the sequence given by SEQ ID NO:1, 3, 5, 7, 9, 11, 13, 15, 17, 19, 21, 23, 25 and 51; and
d) a nucleic acid fragment wherein one or more nucleotides in the nucleotide sequence given by SEQ ID NO:1, 3, 5, 7, 9, 11, 13, 15, 17, 19, 21, 23, 25 and 51 is changed from that given by the chosen sequence to a different nucleotide provided that no more than 15% of the nucleotides are so changed.

10. The nucleic acid molecule of claim 5, wherein said nucleic acid molecule hybridizes under stringent conditions to the nucleotide sequence given by SEQ ID NO:1, 3, 5, 7, 9, 11, 13, 15, 17, 19, 21, 23, 25 and 51, or a complement of said nucleotide sequence.

11. The nucleic acid molecule of claim 5, wherein the nucleic acid molecule comprises a nucleotide sequence in which any nucleotide specified in the coding sequence of the chosen nucleotide sequence is changed from that given by the chosen sequence to a different nucleotide provided that no more than 15% of the nucleotides in the chosen coding sequence are so changed, an isolated second polynucleotide that is a complement of the first polynucleotide, or a fragment of any of them.

12. A vector comprising the nucleic acid molecule of claim 11.

13. The vector of claim 12, further comprising a promoter operably linked to said nucleic acid molecule.

14. A cell comprising the vector of claim 12.

15. An antibody that binds immunospecifically to the polypeptide of claim 1.

16. The antibody of claim 15, wherein said antibody is a monoclonal antibody.

17. The antibody of claim 15, wherein the antibody is a humanized antibody.

18. A method for determining the presence or amount of the polypeptide of claim 1 in a sample, the method comprising:
(a) providing said sample;
(b) introducing said sample to an antibody that binds immunospecifically to the polypeptide; and
(c) determining the presence or amount of antibody bound to said polypeptide, thereby determining the presence or amount of polypeptide in said sample.

19. A method for determining the presence or amount of the nucleic acid molecule of claim 5 in a sample, the method comprising:
(a) providing said sample;
(b) introducing said sample to a probe that binds to said nucleic acid molecule; and
(c) determining the presence or amount of said probe bound to said nucleic acid molecule,
thereby determining the presence or amount of the nucleic acid molecule in said sample.

20. A method of identifying an agent that binds to the polypeptide of claim 1, the method comprising:
(a) introducing said polypeptide to said agent; and
(b) determining whether said agent binds to said polypeptide.

21. A method for identifying a potential therapeutic agent for use in treatment of a pathology, wherein the pathology is related to aberrant expression or aberrant physiological interactions of the polypeptide of claim 1, the method comprising:
(a) providing a cell expressing the polypeptide of claim 1 and having a property or function ascribable to the polypeptide;
(b) contacting the cell with a composition comprising a candidate substance; and
(c) determining whether the substance alters the property or function ascribable to the polypeptide;
whereby, if an alteration observed in the presence of the substance is not observed when the cell is contacted with a composition devoid of the substance, the substance is identified as a potential therapeutic agent.

22. A method for modulating the activity of the polypeptide of claim 1, the method comprising introducing a cell sample expressing the polypeptide of said claim with a compound that binds to said polypeptide in an amount sufficient to modulate the activity of the polypeptide.

23. A method of treating or preventing a pathology associated with the polypeptide of claim 1, said method comprising administering the polypeptide of claim 1 to a subject in which such treatment or prevention is desired in an amount sufficient to treat or prevent said pathology in said subject.

24. The method of claim 23, wherein said subject is a human.

25. A method of treating or preventing a pathology associated with the polypeptide of claim 1, said method comprising administering to a subject in which such treatment or prevention is desired a PTMAX nucleic acid in an amount sufficient to treat or prevent said pathology in said subject.

26. The method of claim 25, wherein said subject is a human.

27. A method of treating or preventing a pathology associated with the polypeptide of claim 1, said method comprising administering to a subject in which such treatment or prevention is desired a PTMAX antibody in an amount sufficient to treat or prevent said pathology in said subject.

28. The method of claim 27, wherein the subject is a human.

29. A pharmaceutical composition comprising the polypeptide of claim 1 and a pharmaceutically acceptable carrier.

30. A pharmaceutical composition comprising the nucleic acid molecule of claim 5 and a pharmaceutically acceptable carrier.

31. A pharmaceutical composition comprising the antibody of claim 15 and a pharmaceutically acceptable carrier.

32. A kit comprising in one or more containers, the pharmaceutical composition of claim 29.

33. A kit comprising in one or more containers, the pharmaceutical composition of claim 30.

34. A kit comprising in one or more containers, the pharmaceutical composition of claim 31.

35. The use of a therapeutic in the manufacture of a medicament for treating a syndrome associated with a human disease, the disease selected from a pathology associated with the polypeptide of claim 1, wherein said therapeutic is the polypeptide of claim 1.

36. The use of a therapeutic in the manufacture of a medicament for treating a syndrome associated with a human disease, the disease selected from a pathology associated with the polypeptide of claim 1, wherein said therapeutic is a PTMAX nucleic acid.

37. The use of a therapeutic in the manufacture of a medicament for treating a syndrome associated with a human disease, the disease selected from a pathology associated with the polypeptide of claim 1, wherein said therapeutic is a PTMAX antibody.

38. A method for screening for a modulator of activity or of latency or predisposition to a pathology associated with the polypeptide of claim 1, said method comprising:
a) administering a test compound to a test animal at increased risk for a pathology associated with the polypeptide of claim 1, wherein said test animal recombinantly expresses the polypeptide of claim 1;
b) measuring the activity of said polypeptide in said test animal after administering the compound of step (a); and
c) comparing the activity of said protein in said test animal with the activity of said polypeptide in a control animal not administered said polypeptide, wherein a change in the activity of said polypeptide in said test animal relative to said control animal indicates the test compound is a modulator of latency of, or predisposition to, a pathology associated with the polypeptide of claim 1.

39. The method of claim 38, wherein said test animal is a recombinant test animal that expresses a test protein transgene or expresses said transgene under the control of a promoter at an increased level relative to a wild-type test animal, and wherein said promoter is not the native gene promoter of said transgene.

40. A method for determining the presence of or predisposition to a disease associated with altered levels of the polypeptide of claim 1 in a first mammalian subject, the method comprising:
a) measuring the level of expression of the polypeptide in a sample from the first mammalian subject; and
b) comparing the amount of said polypeptide in the sample of step (a) to the amount of the polypeptide present in a control sample from a second mammalian subject known not to have, or not to be predisposed to, said disease,
wherein an alteration in the expression level of the polypeptide in the first subject as compared to the control sample indicates the presence of or predisposition to said disease.

41. A method for determining the presence of or predisposition to a disease associated with altered levels of the nucleic acid molecule of claim 5 in a first mammalian subject, the method comprising:
a) measuring the amount of the nucleic acid in a sample from the first mammalian subject; and
b) comparing the amount of said nucleic acid in the sample of step (a) to the amount of the nucleic acid present in a control sample from a second mammalian subject known not to have or not be predisposed to, the disease;
wherein an alteration in the level of the nucleic acid in the first subject as compared to the control sample indicates the presence of or predisposition to the disease.

42. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising the amino acid sequence given by SEQ ID NO:2, 4, 6, 8, 10, 12, 14, 16, 18, 20, 22, 24, 26 and 52 or a biologically active fragment thereof.

43. A method of treating a pathological state in a mammal, the method comprising administering to the mammal the antibody of claim 15 in an amount sufficient to alleviate the pathological state.
